SEQUENCE LISTING (57 sequences)

SEQ ID NO: 1 - DNA, 783 bp, Homo sapiens
agctcctaggtccctgtcctgtggaaatttgtggaccctgggcaccctctcttgctcccaaattttaatcggctcctggaaacctcaccccaaattggagataggcactcctcttgtagaacaaaaggctcaggttcagggagtgagggcctgaactgtgcccccaccctccaggaagggtccttcacggcctggctgcagggatcagtcacgtgtggcccttcattaggccctgccatataagccaagggcacggggtggccgggaactctctaggcaagaatcccggaggcagaggccatgctgaccgcagcggtgctgagctgtgccctgctgctggcactgcctgccacgcgaggagcccagatgggcttggcccccatggagggcatcagaaggcctgaccaggccctgctcccagagctcccaggcctgggcctgcgggccccactgaagaagacaactgcagaacaggcagaagaggatctgttgcaggaggctcaggccttggcagaggtactagacctgcaggaccgcgagccccgctcctcacgtcgctgcgtaaggctgcatgagtcctgcctgggacagcaggtgccttgctgtgacccatgtgccacgtgctactgccgcttcttcaatgccttctgctactgccgcaagctgggtactgccatgaatccctgcagccgcacctagctggccaacgtcagggtcggggctagggtaggggcaaggaaactcgaataaaggatgggaccaacaaaaaaaaaaaaaaaaaaa

SEQ ID NO: 2 - DNA, 4760 bp, Homo sapiens
aaaatcccacgtgactggctctcctctcaggccatcatggcgtctcccagtgggaagggagcccgggcgctggaggctcctggctgcgggccccggccgctcgcccgggacctggtggactccgtggacgatgcggaggggctgtacgtggctgtggagcgctgcccgctgtgcaacactacccgccggcggctgacctgcgccaaatgcgttcagagcggcgatttcgtctacttcgacggccgcgaccgggagaggtttatcgacaagaaggaaaggttaagccgacttaagagcaagcaagaagaatttcagaaagaagtgttaaaagctatggaaggaaaatggataacagatcagttgagatggaaaataatgtcctgcaagatgaggattgaacagttaaaacaaacaatatgtaaaggaaatgaagaaatggagaaaaattctgaaggccttctcaaaaccaaggaaaagaatcagaagctttacagtcgagcacaacggcaccaagagaaaaaggagaagattcagaggcataatcgcaaacttggtgacctggtagaaaaaaagaccattgacttaagaagtcattatgagcgtctggcaaatcttcgacgatcccatatattagagctcacctctgtcatttttccaatcgaggaagtaaagacgggtgtgagagaccccgcagatgtgtcttcagagagtgacagtgccatgacctccagcactgtgagcaagcttgctgaagcccggaggacaacttacctctcaggacgatgggtctgtgacgatcacaacggagacaccagcattagcattacagggccttggattagcctccctaacaatggggactactctgcctactacagctgggtggaggagaagaaaacaacccaggggcctgacatggagcagagtaaccctgcctacaccatcagtgctgcgctgtgctatgcaactcagctggtcaacattctgtctcatatacttgatgtaaatcttcccaaaaagctctgcaacagtgaattttgtggcgaaaatctaagcaagcagaaatttactcgagcagtgaagaaactgaatgcaaatattctttacctttgtttttctcagcatgtaaatttagatcaattacaaccactgcataccctcaggaatctaatgtacctggtcagtccaagctctgaacacctaggcaggtcagggccctttgaagtacgagcagaccttgaggagtccatggaatttgtggatcccggagttgctggagaatcagatgagagcggagatgagcgcgtcagcgatgaagaaaccgacctgggcacagactgggagaacttgcctagtccccggttttgtgatatcccttcccagtctgtggaagtctcccagagtcagagcacccaggcgtccccacccatcgcgagcagcagtgcaggtgggatgatctcctctgcagcagcctcggtgacctcctggtttaaagcttacactggacaccgttaacgagcatggaccaaaacataccaaatctgcatcaagaaagttcttctcccactacactctagtaaacattttctgtttaagttaagatagtgtctggaacaaagaggttaaagtgttgttttgttttgtctttttaagcagggagacaaacatttctatttgccaagtggcctgtgatggtgaccaacatgcttatgataattaagagaacaggggtcgaaggtctttctacccagaccagtgctggtggaaggaggacctgtgcgtgtggccagttctgccaaggaagcagttgatttgggttccctctgggcccgggccaccgggcccacagatatgggtcagtgtgctggtccttgcggtgctgagactgttcctgacactttaagttttagaggttggttgaatcacaagaggtgattcttgattattaggacatgaaagataaaagctctttaataagagtttttctgccattgttttttgtatgagaaccagcaggcaatttaaaatttctaatttggtcctttgattttgtttgggaggggtgagttacacgtattttattcatgctgctctgtcgtagtttgtcagacattcctgtttttctttcccccacacaccaaagaaaatgaaagtctttttctttaggacccacatccataaatggaagaaatcctggctgcaataatgtctagagagtttttaactattttcttgtattctgaggggaattaagcttattcttacctagttgaattcctgccatccacactatgagcattttgaaattgaacttatattttctgggtgaaaataagtcatgaaggtcattcccttatgtaagctcaatgcctgcctgggcacaggggaaaagccacttagttaagtggcctctggtcattcttgtggtgtccactttctttctatgggattgagtaggtggcaggtgttttcaggggaaaccatcctacttgtttccccgaactctttgttgctctgaggacacagctttgctcagaaatgcagcgcagatccttacggctgatgctactctgctctgttctggggaaagcacaatataaagaaagaatttcccagccaggcgcagtggctcacgcctgtaatcccagcactttaggaggccgaggcaggcggatcacttgaggtcaggagtttgagagcagcctggctaacatggtgaaaccctgtttctactaaaaatacaaaaaattaccgggtgtggtggcgcacgcctgtaatcccagctactcgggaggctgcggcaggagaatcgcttgaaccgggaggcagaggttgcagtgagccgagattgtgccattgcactccagcctgggcaacaagagcgaaactccgtctcaaaaaaaaaaagaatttccctcagcaggagatcattttcagctcacgtgtcttgtcattcttttagtgacaatcttacaagaaaactataatgagagaggcattatgtacaaatatgtaagtagtttatttttaataactgcaaaaaaatcctatgtaacaactaccaaaagaaatcctatgaaagagtcctaacaggcattattaccatatcttatgtgattggcatgatagcacctctgataaatcattcagaggtttgccatgccccagcttcttttctcatcataataattgtagttgatactttgcctccaagtccgaggtgctatatagcttttgctaatggtatatttggtgttttgtatagttttgggtagagttgcagaacggagtttatttctatccggtagtcacaaattccttggctctatgaattttccatgaaaggaggaagtaggcttttctcgttgtgggtggtcttttttttttttggagacggagtctcactcagctgcccaggctggagtgtagtggcaccatctccgctcactgcaaccaccatctcctgggttcaagcaattcttccatctcaacctcccgagtagctgggattataggcacctgccatcatgcccagctaatttttgtattttagtaaagacgggggttttcaccatgttggccaggctggtcttgaactcctgacttcaggtgatccgcttgccttggcctcctaaagtgctaggattacaggcctgagccaccgcgcccggccccttatgggttcttctacactgctgggatctctgttttaagtgctcagcttcatgattgattgctgggcttccattttcccatccagttctggagttcgtagagagtgaagatggtagacttgaacagataaataaacttaacgatcttgtaagagttgtctagctacttaaaaccctcagaagtaagagcttagtctcacgagttgtaagagtgggatttggagcttggtggtggagactgacttcagctgagagatgcacaacagtcatggttttcttaagcctcttatgaaaccatgaatgagagatgaagctaaagaatagaatccagagatcacaaactcatctagagtacttccacaaaatttacaaagatgtgggaactttatggataggatatattttgtttgttgttgttaatatcaactagaggcactttacatagggttaagtgatcgaacccttttgtggttttgaacaccaacatactggcttacactgctgaaatattttgggtttcattattttgcactggatccaccctgtaaatactcttaagtatacatttcaaccactgttttttctactctttttgctgctcattaaaatctttcatgtaggtgccagaaccatatgtaaacagctttttaaaaaattgaagctggtattttgtttaaacaaaaagccatagaacttggtcatgttttccattttaaaatgatttactgaaacaaagtaatactaataaaaacccacaggcaccaaacaggctgcttaaaatggtctgttaaagacattttttggttatggaatataagaaaagttttgcacatctgtaagggggaaaaacagtatatcaccattgggtagagtggacgggactcatgtaaggactcaatttggggaagagcattcagtggcatgctgttagaggactagtgtccgagaatctcctcacagtatcatgttgcaggaattccccattgctctgcaacttccaaaccagtttgagtcatacaaatgttttctaaacttttattgtattactgcaataaatcttttaacagtaaaaaaaaaaaaaaaaaa

SEQ ID NO: 3 - DNA, 8270 bp, Homo sapiens
aagagctcgcccagctctgcgggcgccgccaccttcgccgccaccgctgcctttctcctcctcctgtcggcgtgcgggggccgcgcccggcggcagctctgccctaggtgggcggcggcgcggcccaggctgcagctgagcgctctgcgcggcgcagccgggtctcccgcgtgtaccacgccgtgacaggtgcagagtccgggctgaggacccacctgcagccgccgccgcgatgcccaccatgcggaggaccgtgtcggagatccgctcgcgcgccgaaggttatgagaagacagatgatgtttcagagaagacctcactggctgaccaggaggaagtaaggactattttcatcaaccagccccagctgacaaaattctgcaataaccatgtcagcactgcaaaatacaacataatcacattccttccaagatttctctactctcagttcagaagagctgctaattcattttttctctttattgcactgctgcagcaaatacctgatgtgtcaccaacaggtcgttatacaacactggttcctctcttatttattttagctgtggcagctatcaaagagataatagaagatattaaacgacataaagctgataatgcagtgaacaagaaacaaacgcaagttttgagaaatggtgcttgggaaattgtccactgggaaaaggtggcagtaggggagatagtgaaagtgaccaatggggaacatctcccagcagatctcatcagtctgtcctcaagtgagccccaagccatgtgctacattgaaacatccaacttagatggtgaaacaaacttgaaaattagacagggcttaccagcaacatcagatatcaaagacgttgacagtttgatgaggatttctggcagaattgagtgtgaaagtccaaacagacatctctacgattttgttggaaacataaggcttgatggacatggcaccgttccactgggagcagatcagattcttcttcgaggagctcagttgagaaatacacagtgggttcatggaatagttgtctacactggacatgacaccaagctgatgcagaattcaacaagtccaccacttaagctctcaaatgtggaacggattacaaatgtacaaattttgattttattttgtatcttaattgccatgtctcttgtctgttctgtgggctcagccatttggaatcgaaggcattctggaaaagactggtatctcaatctaaactatggtggcgctagtaattttggactgaatttcttgaccttcatcatccttttcaacaatctcattcctatcagcttattggttacattagaagttgtgaaatttacccaggcatacttcataaattgggatcttgacatgcactatgaacccacagacactgctgctatggctcgaacatctaatctgaatgaggaacttggccaggttaaatacatattttctgacaaaactggtactctgacatgcaatgtaatgcagtttaagaagtgcaccatagcgggagttgcttatggccatgtccctgaacctgaggattatggctgctctcctgatgaatggcagaactcacagtttggagatgaaaaaacatttagtgattcatcattgctggaaaatctccaaaataatcatccaactgcacctataatatgtgaatttcttacaatgatggcagtctgtcacacagcagtgccagagcgagaaggtgacaagattatttatcaagcagcatctccagatgagggagcattggtcagagcagccaagcaattgaattttgttttcactggaagaacacccgactcggtgattatagattcactggggcaggaagaaagatatgaattgctcaatgtcttggagtttaccagtgctaggaaaagaatgtcagtgattgttcgcactccatctggaaagttacgactctactgcaaaggagctgacactgtaatttatgatcgactggcagagacgtcaaaatacaaagaaattaccctaaaacatttagagcagtttgctacagaagggttaagaactttatgttttgctgtggctgagatttcagagagcgactttcaggagtggcgagcagtctatcagcgagcatctacatctgtgcagaacaggctactcaaactcgaagagagttatgagttgattgaaaagaatcttcagctacttggagcaacagccattgaggataaattacaagatcaagtgcctgaaaccatagaaacgctaatgaaagcagacatcaaaatctggatccttacaggggacaagcaagaaactgccattaacatcggacactcctgcaaactgttgaagaagaacatgggaatgattgttataaatgaaggctctcttgatggaacaagggaaactctcagtcgtcactgtactacccttggtgatgctctccggaaagagaatgattttgctcttataattgatgggaaaaccctcaaatatgccttaacctttggagtacgacagtatttcctggacttagctttgtcatgcaaagctgtcatttgctgtcgggtttctcctcttcaaaaatctgaagttgttgagatggttaagaaacaagtcaaagtcgtaacgcttgcaatcggtgatggagcaaatgatgtcagcatgatacagacagcgcacgttggtgttggtatcagtggcaatgaaggcctgcaggcagctaattcctctgactactccatagctcagttcaaatatttgaagaatttactgatgattcatggtgcctggaactataacagagtctccaagtgcatcttatactgcttctacaagaatatagtgctctatattatcgagatctggtttgcctttgttaatggcttttctggacagatcctctttgaaagatggtgtataggtctctataacgtgatgtttacagcaatgcctcctttaactcttggaatatttgagagatcatgcagaaaagagaacatgttgaagtaccctgaattatacaaaacatctcagaatgccctggacttcaacaccaaggttttctgggttcattgtttaaatggcctcttccactcagttattctgttttggtttccactaaaagcccttcagtatggtactgcatttggaaatgggaaaacctcggattatctgctactgggaaactttgtgtacacttttgtggtgataactgtgtgtttgaaagctggattggagacatcatattggacatggttcagccacatagcgatatgggggagcatcgcactctgggtggtgttttttggaatctactcatctctgtggcctgccattccgatggcccctgatatgtcaggagaggcagccatgttgttcagttctggagtcttttggatgggcttgttattcatccctgtggcatctctgctccttgatgtggtgtacaaggttatcaagaggactgcttttaaaacattggtcgatgaagttcaggagctggaggcaaaatctcaagacccaggagcagttgtacttggaaaaagcctgaccgagagggcgcaactgctcaagaacgtctttaagaagaaccacgtgaacttgtaccgctctgaatccttgcaacaaaatctgctccatgggtatgcgttctctcaagatgaaaatggaatcgtttcacagtctgaagtgataagagcatatgataccacgaaacagaggcccgacgaatggtgatggggagagcctgaaaggcaggctctgttacctctctaaggagagctaccaggttgtcaccgcagtctgctaaccaattccagtctggtccatgaagaggaaaggtagatctgagctcatctcgctgatggacattcagattcatgtatattatagacataagcactgtgcaactgtactgtaacaccatctcttttggatttttttaaggtatttgctaagtctttgtaaacggaaattgaaaatgacctggtatcttgccagagggctttcttaaacggagaataagtcagtattcttatgccattactgtggggctgtaactgactgtcagtttattggctgtaccacaaggtaaccaaccattaaaaaactctaaatgatatttagttaaagggactcttggtatccagacttagatttcaggatatgctgaaacaaaccagcattcttaaggaactgactcaccttcctgagcaaaatttctaaacaagcatttgtgtccaaaattgtcttgataaatgtttgccaaagaggttcagtaagtgtttttctagttcagtagtcatatgcccagaaatgtaagagaaagtttacttccagttccgctgtaagatctgcatgcctgactttccaaatgtaagagtgatttacaaaaatgaatatttcaaggcatttgctactaaaatcggtgatgttgcacctttggccttacaaatgcttctttgttgtttgtcgtgtttatttgttagaggacacacgtgttaatgtgactctgttgttatgacactgatttttcaaactatgtatgtttcaggtatttctgatgaagtttcatcatcatttagatttttctaaaaatctggctaatgcagtagattgagtgatgtcattttgtcttaaagtttttcctcttaagaaacatatgctacgtatttacgtgggatttccaaagcttctgttgcaatatttggaataacatgtcagataaatgcatgggcttttgtcctgtgttccagttcccactagagatgcctgtgtcttgtgtagcacacccagtgttatggtgactgccccctatactgaagactgaaaattatttcacagttcactcatcaaatagttcccaaaattcgtcacatgctgcttattgggacaaataggtagtacattttccccatttaaaaaatgcggattttactcaggccggtaactttacagtcagaggacacgttcatcatgagtagcttttgttagtatgttttaaaatgtatcttcagttcaattattttcagcatttacaagacatctgaaaatggctattttgctaccaacagtaaatgaaggggctgtttaaaaaccacaaccagttttctacactattttttaaataatactttcatttgaaaaaaaggaattagttttcagatacacttcagagattgaagcaaactatttgccttttactcaaaagcctgcttgcctttacatggacttaccagcaaaataggtagaactttctcttttaaaaaaagtcaactagaattgagaagaggtgattttttttcagatcgcttctcgagtttaatattttcacattcttttcaccctttttctcaatctagatttaaaattaggatatatgtcatttccttgtctgtatttgtagctccttagttaccagtatgcctctccattttctacaaataagaggttataacacatatacataattctaaccttaagggaacacacgtttacatactttacttcccaagcccttcctgtttggggtacagattgagagagtcatgaatcaacacatctagcaagaccacaggtgtaagagtctaagatcgtcttcaaaattctgaagtcccagtctttacctgtccagtgaatgaatattcagagcagcttttcctgggcttcccagtggtgatagctgaggtcaaaccacaaaaaataagaaagcaagagtgaaatgcacccctccagagaaacactttgtagtgtttaattctgttaatagagaagagctgcttctgtttgcgctcacttcatcagtggcacccttctgcagaattttaatataaaaacattatggatataatagaactggattttctgacttaaaaatgtaagttttattttaatcttgaaacgtggattgtttctgtggagctcttaaacatgagaagaatacttacggttgataatgtgtaacatgatctgaaatgtgactaatttgagcctctttgtcccatcgtcctgtttttgaattattgacattgtcagtctctttgcttcctgggtgagacttggggtttgagggacagggaatgaccttcttggtgaaacttaaaatataacattgcaattgcagtgactttacagtgttaaattagagaaaatagtctgattttttaaaccttccttaactggaaaaaagtcacatggttttaccaggattgaaataaacagtcaatgtgacttttaacatgtgtttttttgaaataaagggcacgtactcttcaattaaaaagttccttatagggactctggcaaatgctaacacagttgctttacaatgtttacaattcagacaatacgacttataatagaaaatcctcattcatttagcattgaaaagctggaagttgcttctttaatgttgaatagtatacagtggtattgagcatggactttctaaatgttttatatatacatataaaaatatattggtgtctcacacccagaaagatgttatattgtagatattattaggaaaacagtgtttctcaggaacgttgtaaattttaaatgatatatgtacttcccgtcctcccacctccactctgtgctctaatgtgagactgcttcagcagtgttgctaagttaatggaaaactttttctaatcaagtcaggtgaatgtgtattctgctaaataatgttagccatttacatgaattgtatggtcattaaatggaatcagtgattcctctttaatttccagaggggaaatgaattatggaaatcagtcagcattctgatcattaaattttatactttaattttgccgttcagcattctaaatatccaatgtgaaagtcacatgataatttgttttgcattgcgtgcactgtacaacacttacaacttgtcatttaaaatgttttctcgggaaatgaatgctagtcagaaagtaatagattgtattattcatagttttaaaattatgacaatgtcataattactacaaagctaaataatcgtgtttatttttgtgcagttgccctttgatagttcctggttttaaaacctattaagtgtataatcttacaaatagtcatctacaaaatttatggagaaagtgcccagcccattcacatcacatggaccaggaattcttttgtaaatgacttaaggtaacatcatgcagttcagtgcctaataaatgctttttaatgatgaacatttctataatgactcgtaagataccatagtctgatttttctcacattaaaataactgaagtcacttgtgtaacgtagttatactttgctgcattttaattaaccttcaacagctattaaagtggaatgtaagttaaattttgaaggaaaggaaataaatgttttccatatttcgtcttgatttactttctgtatgagaacagctgtgtttttgataggtttatggtttgcatgagttcatatttaaagtgatccaggccaatgcatggctattgctgtaaatcttgatgtttatttctgccttgtaaagttctatcacggcctacctggaatttaaaattcagtagacaaattaattggtcctctgcacaacttttttaataagtagattattttacaaagaaatttgaacaaatttaattgaatcttttgtttagcttgcctctaagaacttttcttaataaagctcccaaaacttctcagcaaataaatctcccttaagtaggaaagctagatttcatatttgcttactttgaattaacagcaactttccacaggtaaatctgttcttgcaaagatgtgagcagaatagttaaaaataatatttttatgtttcatggttctaaatggaagccataaatgcagtaaatactatctgttgtttaactactttaatcgtcattttttacattttcaagtttattaggttaagaaaaacagggcagccttggaaggcagctactacagaaaactgcagttttgcgttaaagataaagtagtattttcagctccctgaaaaaccattcctgctgaaactgctgtagaaattgtgaagctgcatgagtggagagtattgaatctgtggttatagtagttttctcaggtttgtttatcttgatgtttgatgcactgtgttttatagttattaaaattgagtaatattatttctatgcagtgttatgtgtcattggccttttgtgaatgtgcatgttttaaactgcaaattttaaacattttgtcctctaattgttattaaaaatgaaataaactttaccattacttaaaa

SEQ ID NO: 4 - DNA, 417 bp, Homo sapiens
atggcaaagcaaccttctgatgtaagttctgagtgtgaccgagaaggtagacaattgcagcctgcggagaggcctccccagctcagacctggggcccctacctccctacagacagagccacaagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtatcggagacgagtttaacgcttactatgcaaggagggtatttttgaataattaccaagcagccgaagaccacccacgaatggttatcttacgactgttacgttacattgtccgcctggtgtggagaatgcattga

SEQ ID NO: 5 - DNA, 3575 bp, Homo sapiens
accatcgtcttgggcccggggagggagagccaccttcaggcccctcgagcctcgaaccggaacctccaaatccgagacgctctgcttatgaggacctcgaaatatgccggccagtgaaaaaatcttgtggctttgagggcttttggttggccaggggcagtaaaaatctcggagagctgacaccaagtcctcccctgccacgtagcagtggtaaagtccgaagctcaaattccgagaattgagctctgttgattcttagaactggggttcttagaagtggtgatgcaagaagtttctaggaaaggccggacaccaggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaaaaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaactggaaaaaaaa

SEQ ID NO: 6 - DNA, 2655 bp, Homo sapiens
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa

SEQ ID NO: 7 - DNA, 789 bp, Homo sapiens
gccaccgcccgcagctgaagcacatccgcagcccggcgcgactccgatcgccgcagttgccctctggcgccatgtccgagaacggagcgcccgggatgcaggaggagagcctgcagggctcctgggtagaactgcacttcagcaataatgggaacgggggcagcgttccagcctcggtttctatttataatggagacatggaaaaaatactgctggacgcacagcatgagtctggacggagtagctccaagagctctcactgtgacagcccacctcgctcgcagacaccacaagataccaacagggcttctgaaacagatacccatagcattggagagaaaaacagctcacagtctgaggaagatgatattgaaagaaggaaagaagttgaaagcatcttgaagaaaaactcagattggatatgggattggtcaagtcggccggaaaatattccccccaaggagttcctctttaaacacccgaagcgcacggccaccctcagcatgaggaacacgagcgtcatgaagaaagggggcatattctctgcagaatttctgaaagttttccttccatctctgctgctctctcatttgctggccatcggattggggatctatattggaaggcgtctgacaacctccaccagcaccttttgatgaagaactggagtctgacttggttcgttagtggattacttctgagcttgcaacatagctcactgaagagctgttagatcctgggccttcgtggctcgagagactagaatcgcagatacgaaaaccccgcagc

SEQ ID NO: 8 - DNA, 2251 bp, Homo sapiens
ctgctgaagcgggaaggaggagctagggctgggggcggagctttcacacgcgcaccctctgttccctccctccctccctcgacacaagcaactgggtctccagccgccactccgggtttatttgtttacaagcggattacgtcagctcctccctctcttccctatctctggacccgcctcctgaactcttttcccgcccctttcggctccgaaccggcttgcgtcacaatggtgcgatattcggattggctggagtcggccatcacgctccagctacgccacttccttttcgtggcactataaagggtgctgcacggcgcttgcatctcttcgcctctcggagctggaaatgcagctattgagatcttcgaatgctgcggagctggaggcggaggcagctggggaggtccgagcgatgtgaccaggccgccatcgctcgtctcttcctctctcctgccgcctcctgtctcgaaaataacttttttagtctaaagaaagaaagacaaaagtagtcgtccgcccctcacgccctctcttcctctcagccttccgcccggtgaggaagcccggggtggctgctccgccgtcggggccgcgccgccgagccccagccgccccgggccgcccccgcacgccgcccccatgcatcccttctacacccgggccgccaccatgataggcgagatcgccgccgccgtgtccttcatctccaagtttctccgcaccaaggggctcacgagcgagcgacagctgcagaccttcagccagagcctgcaggagctgctggcaggtgagcaggggagtcctaagcgttgtttctctgttcttcttttcttctatagaacattataaacatcactggttcccagaaaagccatgcaagggatcgggttaccgttgtattcgcatcaaccataaaatggatcctctgattggacaggcagcacagcggattggactgagcagtcaggagctgttcaggcttctcccaagtgaactcacactctgggttgacccctatgaagtgtcctacagaattggagaggatggctccatctgtgtgctgtatgaagcctcaccagcaggaggtagcactcaaaacagcaccaacgtgcaaatggtagacagccgaatcagctgtaaggaggaacttctcttgggcagaacgagcccttccaaaaactacaatatgatgactgtatcaggttaagatatagtctgtggatggatcatctgatgatgatggataaatttgatttttgctttgggtgggctcctcttggggatggattatggaatttaaaccatgtcacagctgtgaagatctggcacaagatagaatggtaaaaaaaaaaaaaaattttaagtgacagtgccatagtttggacagtacctttcaatgattaattttaatagcctgtgagtccaagtaaatgatcactttatttgctagggagggaagtcctagggtggtttcagtttctcccagacatacctaaatttttacatcaatccttttaaagaaaatctgtatttcaaagaatctttctctgcagtaaatctcgcaggggaatttgcactattacacttgaaagttgttattgttaaccttttcggcagcttttaataggaaagttaaacgttttaaacatggtagtactggaaattttacaagacttttacctagcacttaaatatgtataaatgtacataaagacaaactagtaagcatgacctggggaaatggtcagaccttgtattgtgtttttggccttgaaagtagcaagtgaccagaatctgccatggcaacaggctttaaaaaagacccttaaaaagacactgtctcaactgtggtgttagcaccagccagctctctgtacatttgctagcttgtagttttctaagactgagtaaacttcttatttttagaaagtggaggtctggtttgtaactttccttgtacttaattgggtaaaagtcttttccacaaaccaccatctattttgtgaactttgttagtcatcttttatttggtaaattatgaactggtgtaaatttgtacagttcatgtatattgattgtggcaaagttgtacagatttctatattttggatgagaaatttttcttctctctataataaatcgtttcttatcttggcattttaatcaatctctgtcatgatagaggttgctaaagtattttctagagaacggttctataaactgaatatctgttgcacactggtcatgc

SEQ ID NO: 9 - DNA, 2060 bp, Homo sapiens
atttttggccctcgtgacagtgattgatagctgctgggaaggtataaaagcagcttgcctgcgaaggttcttcacactgctcagggaagagcctgctacggtggactgtgagactcagtgcactgtcctcctcccagcgaccccacgctggaccccctgccggaccctccacccttcggcccccaagcttcccaggggcttcctttggactggactgtccctgctcatccattctcctgccacccccagacctcctcagctccaggttgccacctcctctcgccagagtgatgaggtcccggcttctgctctccgtggcccatctgcccacaattcgggagaccacggaggagatgctgcttgggggtcctggacaggagcccccaccctctcctagcctggatgactacgtgaggtctatatctcgactggcacagcccacctctgtgctggacaaggccacggcccagggccaacccaggccaccccacaggccagcccaggcctgccggaagggccgccctgctgtgtccctgcgagacatcaccgcacgtttcagtggccagcagcccacactgcccatggctgatactgtggaccccctggactggctttttggggagtcccaggaaaagcagccaagccagagggacctgccaaggaggactggcccctctgctggcctctggggtccacatagacagatggacagcagcaagcccatgggggcccccagagggaggctctgtgaagccaggatgcctgggcattccctggcaagaccaccgcaggatgggcagcagagctctgacctaagaagctggacttttgggcagtctgcccaagccatggcctcccgccaccgcccccgccccagcagtgtcctcagaacactctactcgcacctcccggtgatccatgaactctgacccctccccagtaaaggcttctgtagagagcatgctgggtctgcatctcctctcgtctcctccatggtggtcactgcccctggcaggtctctgaaagggaaatgcttttctgcagaggcccctgcttgggcagttcacagtgagaccgaccccctctgaatatgataacagcctgtttcacatgaggagatgttaccaatcccgttcgctctgacccttgctggctgatcaccttgagcaacttacttaacatctgtgttcctcagtttctcatgggtaatatagggataattactggcacctgcctcccaggccattctgacgtgtaaccgcatataggagcccactggctgagtagctaccatcatcgctggtggggaaactggtggtaggggtgtgagggtagtgggggtgtcagccccccaggtgtttcagaacaaggcctcgggcactcccaagtctgcctcttggctcccaccctcaaagcccatgttctgtgaggcccaagagaacacatggagtcttagcaaatgcactaatgtattccgggggactgtcacctggcaccactggggcactctgctggctacaactcatacgtcctgtggtggcattgggagagttcccccatgatgagggccaagatagaatctgtaccactcagtgctaccatccccacccctacaccacttccacacaggggcctcatggcatggtcagggtcccagctgtgggtgagagcagggcactgtccagctgtccactggggaagtcaagatgtcctaaggcccaggtcagggcatctggagtctgaaggaccctagttcctagaggcatctggcagcaagaaggtgaggcatcagggaacgggaatcaggctgggactgatcagaggtgaagggacagagagaggagaggaggaagattgagctgggggcaacagccaagctcacctgggcaggtctctgccacctccttgctctgtgagctgtcagtctaggttattctctttttttgtggctatttttaattgctttggatttgttaaatgttttctgtcttctgttaagtgtgttttctctggagatagaatgtaaaccatattaaaaggaaaaagtttcagacaagcaa

SEQ ID NO: 10 - DNA, 2300 bp, Homo sapiens
actcggggcaacaggcagatttgcctgctgagggtggagacccacgagccgaggcctcctgcagtgttctgcacagcaaaccgcacgctatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgacggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctgtgaggccggggccctgcacctgtgcagcgaagcttagcgttcatccgtgtaacccgctcatcactggatgaagattctcctgtgctagatgtgcaaatgcaagctagtggcttcaaaatagagaatcccactttctatagcagattgtgtaacaattttaatgctatttccccaggggaaaatgaaggttaggatttaacagtcatttaaaaaaaaaatttgttttgacggatgattggattattcatttaaaatgattagaaggcaagtttctagctagaaatatgattttatttgacaaaatttgttgaaattatgtatgtttacatatcacctcatggcctattatattaaaatatggctataaatatataaaaagaaaagataaagatgatctactcagaaatttttatttttctaaggttctcataggaaaagtacatttaatacagcagtgtcatcagaagataacttgagcaccgtcatggcttaatgtttattcctgataataattgatcaaattcatttttttcactggagttacattaatgttaattcagcactgatttcacaacagatcaatttgtaattgcttacatttttacaataaataatctgtacgtaagaaca

SEQ ID NO: 11 - DNA, 2723 bp, Homo sapiens
gggagaaacgttctcactcgctctctgctcgctgcgggcgctccccgccctctgctgccagaaccttggggatgtgcctagacccggcgcagcacacgtccgggccaaccgcgagcagaacaaacctttggcgggcggccaggaggctccctcccagccaccgcccccctccagcgcctttttttccccccatacaatacaagatcttccttcctcagttcccttaaagcacagcccagggaaacctcctcacagttttcatccagccacgggccagcatgtctgggggcaaatacgtagactcggagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacccatttaaatttttttccttacctttttatttgcatgtggatcaaccatcgctttattggctgagatatgaacatattgttgaaaggtaatttgagagaaatatgaagaactgaggaggaaaaaaaaaaaaaagaaaagaaccaacaacctcaactgcctactccaaaatgttggtcattttatgttaagggaagaattccagggtatggccatggagtgtacaagtatgtgggcagattttcagcaaactcttttcccactgtttaaggagttagtggattactgccattcacttcataatccagtaggatccagtgatccttacaagttagaaaacataatcttctgccttctcatgatccaactaatgccttactcttcttgaaattttaacctatgatattttctgtgcctgaatatttgttatgtagataacaagacctcagtgccttcctgtttttcacattttccttttcaaatagggtctaactcagcaactcgctttaggtcagcagcctccctgaagaccaaaattagaatatccatgacctagttttccatgcgtgtttctgactctgagctacagagtctggtgaagctcacttctgggcttcatctggcaacatctttatccgtagtgggtatggttgacactagcccaatgaaatgaattaaagtggaccaatagggctgagctctctgtgggctggcagtcctggaagccagctttccctgcctctcatcaactgaatgaggtcagcatgtctattcagcttcgtttattttcaagaataatcacgctttcctgaatccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggagctttggacctaatccaagcatccctttgcccagaaagaagatgggggaggaggcagtaataaaaagattgaagtattttgctggaataagttcaaattcttctgaactcaaactgaggaatttcacctgtaaacctgagtcgtacagaaagctgcctggtatatccaaaagctttttattcctcctgctcatattgtgattctgcctttggggacttttcttaaaccttcagttatgatttttttttcatacacttattggaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataaaaatgtttaacggttaaacagtcaaaaaaaaaaa

SEQ ID NO: 12 - DNA, 3976 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaagtatgtattcttcactactgagtttcttctttggaaaccatcactattgagagatgggaaaaacctgaatgtataaagcatttatttgtcaataaactgccttttgtaaggggttttcacataacata

SEQ ID NO: 13 - DNA, 4304 bp, Homo sapiens
cacacggactacaggggagttttgttgaagttgcaaagtcctggagcctccagagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtgcccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaaccccaaaaaaaaaaaaaaaa

SEQ ID NO: 14 - DNA, 6531 bp, Homo sapiens
gcccagccagcttgcgtcaccgcttcagagcggagaagagcgagcaggggagagcgagaccagttttaaggggaggaccggtgcgagtgaggcagccccgaggctctgctcgcccaccacccaatcctcgcctcccttctgctccaccttctctctctgccctcacctctcccccgaaaaccccctatttagccaaaggaaggaggtcaggggaacgctctcccctccccttccaaaaaacaaaaacagaaaaacccttttccaggccggggaaagcaggagggagaggggccgccgggctggccatggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtgaggatgccagttgggccgaaagagagagacgcgtccataatctggtctcttcttctttctggttgtttttgttctttgtgttttagggtgaaacttaaaaaaaaaattctgcccccacctagatcatatttaaagatcttttagaagtgagagaaaaaggtcctacgaaaacggaataataaaaagcatttggtgcctatttgaagtacagcataagggaatcccttgtatatgcgaacagttattgtttgattatgtaaaagtaatagtaaaatgcttacaggaaaacctgcagagtagttagagaatatgtatgcctgcaatatgggaacaaattagaggagacttttttttttcatgttatgagctagcacatacacccccttgtagtataatttcaaggaactgtgtacgccatttatggcatgattagattgcaaagcaatgaactcaagaaggaattgaaataaggagggacatgatggggaaggagtacaaaacaatctctcaacatgattgaaccatttgggatggagaagcacctttgctctcagccacctgttactaagtcaggagtgtagttggatctctacattaatgtcctcttgctgtctacagtagctgctacctaaaaaaagatgttttattttgccagttggacacaggtgattggctcctgggtttcatgttctgtgacatcctgcttcttcttccaaatgcagttcattgcagacaccaccatattgctatctaatggggaaatgtagctatgggccataaccaaaactcacatgaaacggaggcagatggagaccaagggtgggatccagaatggagtcttttctgttattgtatttaaaagggtaatgtggccttggcatttcttcttagaaaaaaactaatttttggtgctgattggcatgtctggttcacagtttagcattgttataaaccattccattcgaaaagcactttgaaaaattgttcccgagcgatagatgggatggtttatgcaagtcatgctgaatactcctcccctcttctcttttgccccctcccttcctgcccccagtctgggttactcttcgcttctggtatctggcgttctttggtacacagttctggtgttcctaccaggactcaagagacaccccttcctgctgacattcccatcacaacattcctcagacaagcctgtaaactaaaatctgttaccattctgatggcacagaaggatcttaattcccatctctatacttctcctttggacatggaaagaaaagttattgctggtgcaaagatagatggctgaacatcagggtgtggcattttgttcccttttccgtttttttttttttattgttgttgttaattttattgcaaagttgtattcagcgtacttgaatttttcttcctctccacttcttagaggcattcagttagcaaagaggttggagcaacaacttttttttttttttttgcacaattgtaattgacaggtaatgaagctatttgttaaaatatttgcctttttaagtaaaaaagaaaaatcagaacagggctatttgaagaattattttatacacagattctgccttgtttcatagtatgagggttgaagacggaaaacaatctaagggtctctcatttttttaattttgttttgttcagtttggtttttttttttttttgcgctgctaagaagctaaagtcatccatccttattcacgttgacagtacctagctgtaatgtttcacagagtgtgctgctattttataaacatttttataatatattattttactgcttaaattccaagtcctgaagtagatggttgagatatgagttcttcgtactggaaaagcccttccgtagtttgttttcttctggtagcatattcatggttgtttttttttttcttttttggttttttggttttttttttttcctctgatcacattcttcaaagacggagtattctttacctcaggtttactggacaaaatcaataactacaaaaggcaatgattcacgcttttgttttcataatacctcacaaccgtacagtttctgcttgggagcccattcgcatgaggaatacagaagcagtgtgagcagggctgactccctctcaggtggaaggcagggcggtctcactcccagggacctttttggtcatggaggccatcgggctcccagttagaccctggtatcctcatcatgatggaaaaaatacattgaaccaagggatcctccctccccttcaaggcagacgttcagtacaaacatttatgcggtaggctcagatgtcgtaatttgcacttaggtaccaggtgtcaggaaacagactaaaaagaattccaccaggctgtttggagatcctcatcttggagctttttcaaaagcggggcttcatctgcaaagggccctttcatcttgaagtttttcccctccgtctttcccctcccctggcatggacaccttgtgtttaggatcatctctgcaggtttcctaggtctgaatctgcgagtagatgaacctgcagcaagcagcgtttatggtgcttccttctccctcctctgtctcaaactgcgcaggcaagcactatgcaagcccaggccctctgctgagcggtactaaacggtcgggttttcaatcacactgaattggcaggataagaaaaataggtcagataagtatgggatgatagttgaagggaggtgaagaggctgcttctctacagaggtgaaattccagatgagtcagtctcttgggaagtgtgtttagaagggttcaggactttgtgagttagcatgaccctaaaattctaggggatttctggtgggacaatgggtggtgaattctgaagttttggagagggaagtggagcagccagcaagtaagctagccagagttttctcaagagccagctttgctcagcacactctcctgggccccaaggagtcccacggaatggggaaagcgggaaccctggagttcttgggaatcttggagcctaaagagaaaccgaggtgcaaattcatttcatggtgactgacccttgagcttaaacagaagcagcaaatgaaagaaccggacaaataaggaagggcacaagcctacccgactctatttacagtctgtaactttccactcttcctgtagtcccgaggcccctgggtccttctagcttttctctttcccatccttggggccttgtgtgatgatgggtgtggggctgccgatgggaaagtcgggggttgttaggcttttctgcctgctcctgcttaaacacaagaaggaatcctggattttgccctctccttagctcttagtctctttggtaggagttttgttccagaggagctctcccccttggatttgaacttgctctttttgttgttgttgttctttctcttctttttcttacctcccactaaaggggttccaaattatcctggtctttttctaccttgttgtgtttctatctcgtctttacttccatctgtttgtttttttctccatcagtgggggccgagttgttcccccagcctgccaaattttgatccttcccctcttttggccaaatcctagggggaagaaatcctagtatgccaaaaatatatgctaagcataattaaactccatgcgggtccataacagccaagaagcctgcaggagaaagccaagggcagttccctccgcagaacaccccatgcgtgctgagaggcgagctccttgaagaaggggctgttcttccaggaggccttattttgaactgcctcaggaccccactggagagcacagcatgccttactactgggtcatccttggtctatgtgctctgtactggaggctctgttctgcctcttatcagccaggtcaggggcacacatggcttaagtgacaaagccagaggagaagacaaccctgacagcatcacgctgcatcccattgctagcaggattggcaactcttcagacggagctgcgcttccctgcagtctagcacctctagggcctctccagactgtgccctgggagctctgggactgaaaggttaagaacataaggcaggatcagatgactctctccaagagggcaggggaattttctctccatgggccacaggggacagggctgggagaagaaatagacttgcaccttatgtcatgtaaataattgattttctagttcaagaagataatattggtagtgtgggaattggaggtaggaaggggaggaagtctgagtaagccagttggcttctaagccaaaaggattcctctttgtttatctctgagacagtccaaccttgagaatagctttaaaagggaaattaatgctgagatgataaagtccccttaagccaacaaaccctctgtagctatagaatgagtgcaggtttctattggtgtggactcagagcaatttacaagagctgttcatgcagccatccatttgtgcaaaatagggtaagaagattcaagaggatatttattacttcctcataccacatggcttttgatgattctggattctaaacaacccagaatggtcatttcaggcacaacgatactacattcgtgtgtgtctgcttttaaacttggctgggctatcagaccctattctcggctcaggttttgagaagccatcagcaaatgtgtacgtgcatgctgtagctgcagcctgcatcccttcgcctgcagcctactttggggaaataaagtgccttactgactgtagccattacagtatccaatgtcttttgacaggtgcctgtccttgaaaaacaaagtttctatttttatttttaattggtttagttcttaactgctggccaactcttacatccccagcaaatcatcgggccattggattttttccattatgttcatcacccttatatcatgtacctcagatctctctctctctcctctctctcagttatgtagtttcttgtcttggacttttttttttcttttctttttctttttttttttgctttaaaacaagtgtgatgccatatcaagtccatgttattctctcacagtgtactctataagaggtgtgggtgtctgtttggtcaggatgttagaaagtgctgataagtagcatgatcagtgtatgcgaaaaggtttttaggaagtatggcaaaaatgttgtattggctatgatggtgacatgatatagtcagctgccttttaagaggtcttatctgttcagtgttaagtgatttaaaaaaataataacctgttttctgactagtttaaagatggatttgaaaatggttttgaatgcaattaggttatgctatttggacaataaactcaccttgacctaaattaaaaaaaaaa

SEQ ID NO: 15 - DNA, 5489 bp, Homo sapiens
gaaactcttaacaaaaacaaggggctcggggaggtttccgctgaggcggcgggggtgcggcggtgggctggtcttccgcggccggcgttgcgccgcggcggagggtgggcgcgcggggagcgggatggagccggggctgtgaggccgaggcggcggtgcctgggaggaagggtcggatgccggaccgggggcaccgctgaggcggtgggtccccgacctgcgagacaggtttggaagcccccgctgcgcccagtccgtgcggaccgcgaggccgcgggcgggtggaggcgcgtctccggcacgatgaaggatttgggggcagagcacttggcaggtcatgaaggggtccaacttctcgggttgttgaacgtctacctggaacaagaagagagattccaacctcgagaaaaagggctgagtttgattgaggctaccccggagaatgataacactttgtgtccaggattgagaaatgccaaagttgaagatttaaggagtttagccaacttttttggatcttgcactgaaacttttgtcctggctgtcaatattttggacaggttcttggctcttatgaaggtgaaacctaaacatttgtcttgcattggagtctgttcttttttgctggctgctagaatagttgaagaagactgcaatattccatccactcatgatgtgatccggattagtcagtgtaaatgtactgcttctgacataaaacggatggaaaaaataatttcagaaaaattgcactatgaattggaagctactactgccttaaactttttgcacttataccatactattatactttgtcatacttcagaaaggaaagaaatactgagccttgataaactagaagctcagctgaaagcttgcaactgccgactcatcttttcaaaagcaaaaccatctgtattagccttgtgccttctcaatttggaagtggaaactttgaaatctgttgaattactggaaattctcttgctagttaaaaaacattccaagattaatgacactgagttcttctactggagagagttggtttctaaatgcctagccgagtattcttctcctgaatgttgcaaaccagatcttaagaagttggtttggatcgtttcaaggcgcacagcccagaacctccacaacagctactatagtgttcctgagctgccaacgatacctgaggggggttgttttgatgaaagtgaaagtgaggactcttgtgaagatatgagttgtggagaggagagtctcagcagctctcctcccagtgatcaagagtgcaccttctttttcaacttcaaagtggcacaaacactgtgctttccatcttagaaatctgattgttctgtcagaatttatatttacaggtttcaaagcaataaatgggggaataggtagtttcctggtttagcccccatctagtcaggaattaatatactggaatacctaccttctatttgttattcagatcagatctggcctattttcatatttatcctaagccatcaaatggggtagtgcctcttaaaccattaacagtactttagacattggcactttatttttctcgtagatctttagctactttggggaggagggaaggtgctgataccttcaatttgttacttttcaagatttttaaaaataactagtgtagcttatcttaaacattttataaaaccttcagatgtctttaagcagattggaagtatgcaagtgcttccttagcagggacagtggataatccttaatggtttatcatagatttcaccctccccccttctcagaagagtgagtatgctcttaaatgtcaaacacatttttgttgttttgttttttaaatgatcagtgtctatttgatgtgatgcagatcttataaatttgggaattataatattgacatttctgtgatttttatatatgtaatgtcttaattgagatttctgttaaggcagaaataattaggctagggctcttagttttcattcctattgcccaagtattgtcaaactatggtattattttaatgttactttaaaaatccataatctgctagttttgcatgtacttatatgaaaacagtgcagtaagttgaaaactcagtatctatggaattgataaatgttgatctggtgtagtatattttatcgcattttcttatattaaaaaatgtctgcatgattacattttatttcctttgtaatttacatttcagaatagtgtattgctatatgggtgccaagattgaatatgaagaacccgagtgtttgtagtattatagttttaagcaaatctgtgtggtgatacagccataagaatggggcttatataaactctgtacatgtaagattttgtacagagaatttttaactttataaattgtatatgaacatgtaaatcttttaaaatgtacataaaatactgtatttttttaccttgtgtgtgatagtctagtcattgcatgtaaatataatttattatgtattctgtagtataaatcatacattgatgacttacatttttactggtaagtcaacatccgttggatgttttctgaagtggctctttttgaagtgataatagattgtaattcaaaataaaattattaatgaattctccttgtttgggatcacatcttaatttttaatctgttaaaagttcttgatgtattttaatgagaagactttaggtgaggctacagtgattccagagtgagccttctaactggctagcagaagttctctaggtttggcatctgtgccttggagatactgaaagagaatctgtcatttgacaattgacctctttgtgggatggactcattaagtatgctctcagagactggtatattaccagaatgcctattaattttcagtgagaggcaacaggtattaagtagaacagaatgctcaggttggcagattagaacgatctttcaggagacaaagcaagttttaatcagttgtttggttaataagtatggggtgttcgctgtgatagggccccgccagcttctggctcttgtggacctcaaaagtatcaggtggttttgcaagtggtggtcctttcccctgccccaccccaataggttccccatctgtctagtttgatttttgtagacctttgttttctctagttagaaaatcaggtacactgaatatggttttcatgtaacacctcttctctggagataggggtatgttttcctacccttctagtggagaatcctacttgaggatgacctttcctctcttactaaataatattagtaaatagtgggcaatatattctgctttcagattttgatttgttgagatgtaaaagttgtttggggcttaccaaatctcaagactctctttagctcctgcaggattgtattgcttttcttactggatatttttcctgggtaagcatctttgtggcttcatctcttccccctgtggttttcagtgtatttagtcgagacctctctgctgagcttgcaacctgtttattcacatggcctgccatgccacttggaggtttctgattactcccaaacctgctggttctttatgtctttctcagcgaataattccatctattcatgttggaaacttaggtgatatgctcatctccttttgcctgtttatggaggtcaccagcctctatcatttgtatgatttcgtttacactgtttatatctctctgtcccccctttttctgccattggcatggtttagacctgtactctttatcagcagaggtactgtaatatatttgtgatccctcagcttccaggcttactcctggtctctgccttcctatctacatatccttttaaaataaaattttaactatctcctgaaaaattgttgagtaggtcacgcacaatcaggagaaaaatctattcatgacatacaagtctctgtctaatctgaacactgcacctgtctctggcctttttttcttgtcatttcctagaccttaaaaaatgtgtattgagaaagaactctgttagctatacagaagatgaactgggcaatatagagtagcagcatggagaccagtctgactgaactaaggcagtggaagtgtggatgaggaagagaggtgaaaattgagaagcgctatcctttctctttgggcattattaggaggctcacagacaagtccaggagcctggttataccctcctgtgccattcaaccaggtggctttcccatgactgtgatgaataaaattgagaagcccctgcccttttcagagcagagggtgaggagaaagctaccattttgtcctcatccttacccccgttgacttggcgagagatttgacctttcaggttttgatcctgtcattttctaggatgtggtgcacgcactttgctgttgcgcatggtgaagtattgtgcctaggtcctgggtcttcatctgtttggctctgctactgtttcctcctcccaggaagtgtggttagacaaataatgtgttttaattacctgtcacactcaggattaatacatactcaggttaactgtagagaggcattggcttcagaacactcctcgtgacaattttaaccattttctttgtctagagtctgcctttttcttttttacaatttcttttatttcaacactaggtttcaatatggtgttcctgctacctcccacctccctcctccctcatcacacatgcaaattgtcagcttattgagacaacccacttagattcatatatggacaaggacaaggtattttgcatttgttactggaattcagttttcctaactatttactaccagaaatggtcaataacttactttgtgtttagcaaatcaaattgtgtgatagatagtttcccagtatgatggccagtcagtctttccatccctgtgcctacatgctgctcttcccgtccacaagtggagtctgtttctcttgagttttggctggccttatgaatggctttgcttactgaagtgcagcagaagaaatttagtatatgtccaagcctaggctttaagagactggcagctttccttttatcctttttggaagctagccaccatgctgcaaagaagctcagctggattactgaaagatgagaggccatgtggagagagactcttgaggatgagagattatcttggatgttccagccttaagctcccagctgaatgtgggtgtatcctcagctacaccacagaaaacagaggaactactcagtcgatcccaatcaacccacagactcactagaaataacaaattattgttttaagccacgaggttttgggggagggttgttaaacagtaatagataagtgagacagattgcttgttatttatggtcaaatggtgattatctctggtgagattacaggtgatgttttttttaagttatgcctatctgtagtttcctttttttcctaaaattgatttgaattattagtgtattaacagaataaagaatgaactttaaaacacaaaaaaaaaaaaaaaa

SEQ ID NO: 16 - DNA, 2122 bp, Homo sapiens
ggtggctattttgtccttgggctgcctgttttcagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 17 - DNA, 2413 bp, Homo sapiens
cttcttcgtcagcctcccttccaccgccatattgggccactaaaaaaagggggctcgtcttttcggggtgtttttctccccctcccctgtccccgcttgctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggctgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaaacagctcgaattaagaatatgtttccttgtttatcagatacatcactgcttgatgaagcaaggaagatatacatgaaaattttaaaaatacatatcgctgacttcatggaatggacatcctgtataagcactgaaaaacaacaacacaataacactaaaattttaggcactcttaaatgatctgcctctaaaagcgttggatgtagcattatgcaattaggtttttccttatttgcttcattgtactacctgtgtatatagtttttaccttttatgtagcacataaactttggggaagggagggcagggtggggctgaggaactgacgtggagcggggtatgaagagcttgctttgatttacagcaagtagataaatatttgacttgcatgaagagaagcaattttggggaagggtttgaattgttttctttaaagatgtaatgtccctttcagagacagctgatacttcatttaaaaaaatcacaaaaatttgaacactggctaaagataattgctatttatttttacaagaagtttattctcatttgggagatctggtgatctcccaagctatctaaagtttgttagatagctgcatgtggcttttttaaaaaagcaacagaaacctatcctcactgccctccccagtctctcttaaagttggaatttaccagttaattactcagcagaatggtgatcactccaggtagtttggggcaaaaatccgaggtgcttgggagttttgaatgttaagaattgaccatctgcttttattaaatttgttgacaaaattttctcattttcttttcacttcgggctgtgtaaacacagtcaaaataattctaaatccctcgatatttttaaagatctgtaagtaacttcacattaaaaaatgaaatattttttaatttaaagcttactctgtccatttatccacaggaaagtgttatttttcaaggaaggttcatgtagagaaaagcacacttgtaggataagtgaaatggatactacatctttaaacagtatttcattgcctgtgtatggaaaaaccatttgaagtgtacctgtgtacataactctgtaaaaacactgaaaaattatactaacttatttatgttaaaagattttttttaatctagacaatatacaagccaaagtggcatgttttgtgcatttgtaaatgctgtgttgggtagaataggttttcccctcttttgttaaataatatggctatgcttaaaaggttgcatactgagccaagtataattttttgtaatgtgtgaaaaagatgccaattattgttacacattaagtaatcaataaagaaaacttccatagctatt

SEQ ID NO: 18 - DNA, 4372 bp, Homo sapiens
ggtgcctccgggggcggggcctccttcggttggcggcctcgggcttcgggagtcctccaagaggccaggtgaggccgtcccgtgatgccccgcgccccggccgctctggcctgcaacgtgtctctggggcggaggcagcggcagtggagttcgctgcgcgctgttgggggccacctgtcttttcgcttgtgtccctctttctagtgtcgcgctcgagtcccgacgggccgctccaagcctcgacatgtcgtacaactacgtggtaacggcccagaagcccaccgccgtgaacggctgcgtgaccggacactttacttcggccgaagacttaaacctgttgattgccaaaaacacgagattagagatctatgtggtcaccgccgaggggcttcggcccgtcaaagaggtgggcatgtatgggaagattgcggtcatggagcttttcaggcccaagggggagagcaaggacctgctgtttatcttgacagcgaagtacaatgcctgcatcctggagtataaacagagtggcgagagcattgacatcattacgcgagcccatggcaatgtccaggaccgcattggccgcccctcagagaccggcattattggcatcattgaccctgagtgccggatgattggcctgcgtctctatgatggccttttcaaggttattccactagatcgcgataataaagaactcaaggccttcaacatccgcctggaggagctgcatgtcattgatgtcaagttcctatatggttgccaagcacctactatttgctttgtctaccaggaccctcaggggcggcacgtaaaaacctatgaggtgtctctccgagaaaaggaattcaataagggcccttggaaacaggaaaatgtcgaagctgaagcttccatggtgatcgcagtcccagagccctttgggggggccatcatcattggacaggagtcaatcacctatcacaatggtgacaaatacctggctattgcccctcctatcatcaagcaaagcacgattgtgtgccacaatcgagtggaccctaatggctcaagatacctgctgggagacatggaaggccggctcttcatgctgcttttggagaaggaggaacagatggatggcaccgtcactctcaaggatctccgtgtagaactccttggagagacctctattgctgagtgcttgacataccttgataatggtgttgtgtttgtcgggtctcgcctgggtgactcccagcttgtgaagctcaacgttgacagtaatgaacaaggctcctatgtagtggccatggaaacctttaccaacttaggacccattgtcgatatgtgcgtggtggacctggagaggcaggggcaggggcagctggtcacttgctctggggctttcaaggaaggttctttgcggatcatccggaatggaattggaatccacgagcatgccagcattgacttaccaggcatcaaaggattatggccactgcggtctgaccctaatcgtgagactgatgacactttggtgctctcttttgtgggccagacaagagttctcatgttaaatggagaggaggtagaagaaaccgaactgatgggtttcgtggatgatcagcagactttcttctgtggcaacgtggctcatcagcagcttatccagatcacttcagcatcggtgaggttggtctctcaagaacccaaagctctggtcagtgaatggaaggagcctcaggccaagaacatcagtgtggcctcctgcaatagcagccaggtggtggtggctgtaggcagggccctctactatctgcagatccatcctcaggagctccggcagatcagccacacagagatggaacatgaagtggcttgcttggacatcaccccattaggagacagcaatggactgtcccctctttgtgccattggcctctggacggacatctcggctcgtatcttgaagttgccctcttttgaactactgcacaaggagatgctgggtggagagatcattcctcgctccatcctgatgaccacctttgagagtagccattacctcctttgtgccttgggagatggagcgcttttctactttgggctcaacattgagacaggtctgttgagcgaccgtaagaaggtgactttgggcacccagcccaccgtattgaggacttttcgttctctttctaccaccaacgtctttgcttgttctgaccgccccactgtcatctatagcagcaaccacaaattggtcttctcaaatgtcaacctcaaggaagtgaactacatgtgtcccctcaattcagatggctatcctgacagcctggcgctggccaacaatagcaccctcaccattggcaccatcgatgagatccagaagctgcacattcgcacagttcccctctatgagtctccaaggaagatctgctaccaggaagtgtcccagtgtttcggggtcctctccagccgcattgaagtccaagacacgagtgggggcacgacagccttgaggcccagcgctagcacccaggctctgtccagcagtgtaagctccagcaagctgttctccagcagcactgctcctcatgagacctcctttggagaagaggtggaggtgcacaacctacttatcattgaccaacacacctttgaagtgcttcatgcccaccagtttctgcagaatgaatatgccctcagtctggtttcctgcaagctgggcaaagaccccaacacttacttcattgtgggcacagcaatggtgtatcctgaagaggcagagcccaagcagggtcgcattgtggtctttcagtattcggatggaaaactacagactgtggctgaaaaggaagtgaaaggggccgtgtactctatggtggaatttaacgggaagctgttagccagcatcaatagcacggtgcggctctatgagtggacaacagagaaggagctgcgcactgagtgcaaccactacaacaacatcatggccctctacctgaagaccaagggcgacttcatcctggtgggcgaccttatgcgctcagtgctgctgcttgcctacaagcccatggaaggaaactttgaagagattgctcgagactttaatcccaactggatgagtgctgtggaaatcttggatgatgacaattttctgggggctgaaaatgcctttaacttgtttgtgtgtcaaaaggatagcgctgccaccactgacgaggagcggcagcacctccaggaggttggtcttttccacctgggcgagtttgtcaatgtcttttgccacggctctctggtaatgcagaatctgggtgagacttccacccccacacaaggctcggtgctcttcggcacggtcaacggcatgatagggctggtgacctcactgtcagagagctggtacaacctcctgctggacatgcagaatcgactcaataaagtcatcaaaagtgtggggaagatcgagcactccttctggagatcctttcacaccgagcggaagacagaaccagccacaggtttcatcgacggtgacttgattgagagtttcctggatattagccgccccaagatgcaggaggtggtggcaaacctacagtatgacgatggcagcggtatgaagcgagaggccactgcagacgacctcatcaaggttgtggaggagctaactcggatccattagccaagggcagggggcccctttgctgaccctccccaaaggctttgccctgctgccctccccctcctctccaccatcgtcttcttggccatgggaggcctttccctaagccagctgcccccagagccacagttcccctatgtggaagtggggcgggcttcatagagacttgggaatgagctgaaggtgaaacattttctccctggatttttaccagtctcacatgattccagccatcaccttagaccaccaagccttgattggtgttgccagttgtcctccttccggggaaggattttgcagttctttggctgaaaggaagctgtgcgtgtgtgtgtgtgtatgtgtgtgtgtgtatgtgtatctcacactcatgcattgtcctctttttatttagattggcagtgtagggagttgtgggtagtggggaagagggttaggagggtttcattgtctgtgaagtgagaccttccttttacttttcttctattgcctctgagagcatcaggcctagaggcctgactgccaagccatgggtagcctgggtgtaaaacctggagatggtggatgatccccacgccacagcccttttgtctctgcaaactgccttcttcggaaagaagaaggtgggaggatgtgaattgttagtttctgagttttaccaaataaagtagaatataagaagaaaggtaaaaaaaaaaaaaaaaa

SEQ ID NO: 19 - DNA, 827 bp, Homo sapiens
gtgatccttggggccaggtatggcatgcccattgatatgtggagcctgggctgcattttagcagagctcctgacgggttaccccctcttgcctggggaagatgaaggggaccagctggcctgtatgattgaactgttgggcatgccctcacagaaactgctggatgcatccaaacgagccaaaaattttgtgagctccaagggttatccccgttactgcactgtcacgactctctcagatggctctgtggtcctaaacggaggccgttcccggagggggaaactgaggggcccaccggagagcagagagtgggggaacgcgctgaaggggtgtgatgatccccttttccttgacttcttaaaacagtgtttagagtgggatcctgcagtgcgcatgaccccaggccaggctttgcggcacccctggctgaggaggcggttgccaaagcctcccaccggggagaaaacgtcagtgaaaaggataactgagagcaccggtgctatcacatctatatccaagttacctccaccttctagctcagcttccaaactgaggactaatttggcgcagatgacagatgccaatgggaatattcagcagaggacagtgttgccaaaacttgttagctgagctcacgtcccctgatgctggtaacctgaaagatacgacattgctgagccttactgggttgaaaaggagtagctcagacctgtttttatttgctcaataactctactcatttgtatcttttcagcacttaattttaatgtaagaaagttgttcattttgtttttataaaatacatgaggacaatgaaaaaaaaaaaa

SEQ ID NO: 20 - DNA, 4975 bp, Homo sapiens
ctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggggggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 21 - DNA, 4627 bp, Homo sapiens
tcacttgcctgatatttccagtgtcagagggacacagccaacgtggggtcccttctaggctgacagccgctctccagccactgccgcgagcccgtctgctcccgccctgcccgtgcactctccgcagccgccctccgccaagccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtgtgaagtgggttatactggtgtccgatgtgaacacttctttttaaccgtccaccaacctttaagcaaagagtatgtggctttgaccgtgattcttattattttgtttcttatcacagtcgtcggttccacatattatttctgcagatggtacagaaatcgaaaaagtaaagaaccaaagaaggaatatgagagagttacctcaggggatccagagttgccgcaagtctgaatggcgccatcaaacttatgggcagggataacagtgtgcctggttaatattaatattccattttattaataatatttatgttgggtcaagtgttaggtcaataacactgtattttaatgtacttgaaaaatgtttttatttttgttttatttttgacagactatttgctaatgtataatgtgcagaaaatatttaatatcaaaagaaaattgatatttttatacaagtaatttcctgagctaaatgcttcattgaaagcttcaaagtttatatgcctggtgcacagtgcttagaagtaagcaattcccaggtcatagctcaagaattgttagcaaatgacagatttctgtaagcctatatatatagtcaaatcgatttagtaagtatgttttttatgttcctcaaatcagtgataattggtttgactgtaccatggtttgatatgtagttggcaccatggtatcatatattaaaacaataatgcaattagaatttgggagaagcaaatataggtcctgtgttaaacactacacatttgaaacaagctaaccctggggagtctatggtctcttcactcaggtctcagctataattctgttatatgaggggcagtggacagttccctatgccaactcacgactcctacaggtactagtcactcatctaccagattctgcctatgtaaaatgaattgaaaaacaattttctgtaatcttttatttaagtagtgggcatttcatagcttcacaatgttccttttttgtatattacaacatttatgtgaggtaattattgctcaacagacaattagaaaaaagtccacacttgaagcctaaatttgtgctttttaagaatatttttagactatttctttttataggggctttgctgaattctaacattaaatcacagcccaaaatttgatggactaattattattttaaaatatatgaagacaataattctacatgttgtcttaagatggaaatacagttatttcatcttttattcaaggaagttttaactttaatacagctcagtaaatggcttcttctagaatgtaaagttatgtatttaaagttgtatcttgacacaggaaatgggaaaaaacttaaaaattaatatggtgtatttttccaaatgaaaaatctcaattgaaagcttttaaaatgtagaaacttaaacacaccttcctgtggaggctgagatgaaaactagggctcattttcctgacatttgtttattttttggaagagacaaagatttcttctgcactctgagcccataggtctcagagagttaataggagtatttttgggctattgcataaggagccactgctgccaccacttttggattttatgggaggctccttcatcgaatgctaaacctttgagtagagtctccctggatcacataccaggtcagggaggatctgttcttcctctacgtttatcctggcatgtgctagggtaaacgaaggcataataagccatggctgacctctggagcaccaggtgccaggacttgtctccatgtgtatccatgcattatataccctggtgcaatcacacgactgtcatctaaagtcctggccctggcccttactattaggaaaataaacagacaaaaacaagtaaatatatatggtcctatacatattgtatatatattcatatacaaacatgtatgtatacatgaccttaatggatcatagaattgcagtcatttggtgctctgctaaccatttatataaaacttaaaaacaagagaaaagaaaaatcaattagatctaaacagttatttctgtttcctatttaatatagctgaagtcaaaatatgtaagaacacattttaaatactctacttacagttggccctctgtggttagttccacatctgtggattcaaccaaccaaggacggaaaatgcttaaaaaataatacaacaacaacaaaaaatacattataacaactatttacttttttttttttctttttgagatggagtctcgctctgttgcccaggttggagtgcagtggcacgatctcggctcactgcaacctcacctcccgggttcaagagatcctcctgcctcagcctcctgagcagctgggactacaggcgcatgccaccatgcccagctaatttttgtatttttagtagaggcggggtttcaccatgttggccaggatggtctcaatctcctaaccttgagatccaccctccacagcctcccaaactgctgggattacaggcgtgagccaccgcacgtagcatttacattaggtattacaagtaatgtaaagatgatttaagtatacaggaggatgtgaataggttatatgcaagcactatgcccttttatataagtgacttgaacatctgtgcccgattttagtatgtgcaggggggcgatctgggaatcagtcccctgtggataccaaggtacaactgtatttattaacgcttactagatgtgaggagagtctgaatattttcagtgatcttggctgtttcaaaaaaatctattgacttttcaataaatcagctgcaatccatttatttcatttacaaaagatttattgtaagcctctcaatcttggtttttcagttgatcttaagcatgtcaattcataaaaacaagtcatttttgtatttttcatctttaagaatgcttaaaaaagctaatccctaaaatagttagatctttgtaaatgcatattaaataataaagtatgacccacattactttttatgggtgaaaataagacaaaaataatagttttagtgaggatggtgctgagtaaacataaaaactgatttgctctcagctgatgtgtcctgtacacagtgggaagattttagttcacacttagtctaactcccccattttacagatttctcactatatatatttctagaaggggctatgcatattcaatgtattgagaaccaaagcaaccacaaatgcataaatgcataatttatggtcttcaaccaaggccacataataacccagttaacttactctttaaccaggaatattaagttctataactagtactcaaggtttaaccttaaaattaagatttccttaaccttaaccttaaaattgatattatattaaacatacataatacaatgtaactccactgttctcctgaatattttttgctctaatctctctgccgaaagtcaaagtgatgggagaattggtatactggtatgactacgtcttaagtcagatttttatttatgagtctttgagactaaattcaatcaccaccaggtatcaaatcaacttttatgcagcaaatatatgattctagtgtctgacttttgttaaattcagtaatgcagtttttaaaaacctgtatctgacccactttgtaatttttgctccaatatccattctgtagacttttgaaaaaaaagtttttaatttgatgcccaatatattctgaccgttaaaaaattcttgttcatatgggagaagggggagtaatgacttgtacaaacagtatttctggtgtatattttaatgtttttaaaaagagtaatttcatttaaatatctgttattcaaatttgatgatgttaaatgtaatataatgtattttctttttattttgcactctgtaattgcactttttaagtttgaagagccattttggtaaacggtttttattaaagatgctatggaacataaagttgtattgcatgcaatttaaagtaacttatttgactatgaatattatcggattactgaattgtatcaatttgtttgtgttcaatatcagctttgataattgtgtaccttaagatattgaaggagaaaatagataatttacaagatattattaatttttatttatttttcttgggaattgaaaaaaattgaaataaataaaaatgcattgaacatcttgcattcaaaatcttcactgac

SEQ ID NO: 22 - DNA, 6450 bp, Homo sapiens
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 23 - DNA, 3376 bp, Homo sapiens
ggcgaccgaacgcggcggtcggcagcgttcgcgcgggggcctgcgaagcgctgctcggggccggcactgcccgcggggaggacgcgccgccgccgccacccagcgccgccgccgccgccgcctccagccgggccgccgcgcgtcccgggggccggccccgcgagcgcaggagtaaacaccgccggagtcttggagccgctgcagaagggaataaagagagatgcagggatttgtgaggttacggcgccccagctgcaagatgcactagccggctgaacccgggatcggctgacttgttggaaccggagtgctctgcacggagagtggtggatgagttgaagttgccttcccggggctcattttccacgctgccgagaggaatccgagaggcaaggcaatcacttcgtcttgccattgattgggtatcgggagctttttttttctcccctctctctttcttttcctccgtcttgttgcatgcaagaaaattacagtccgctgctcgcccgccctgggtgcgagatattcagccccgctctctcccgtgcattgtgcaacccaaagatgaaagaccgaaggggagaaagttaaagaaatcgcccacatgcgctggatcagtccacggcttggggaaaggcatccagagaaggtgggagcggagagtttgaagtctttacaggcgggaagatggcggactggagctgaaagtgttgattgggaaacttgggtgattcttgtgtttatttacaatcctcttgacccaggcaggacacatgcaggccaaaaaacgctatttcatcctgctctcagctggctcttgtctcgcccttttgttttatttcggaggcttgcagtttagggcatcgaggagccacagccggagagaagaacacagcggtaggaatggcttgcaccaccccagtccggatcatttctggccccgcttcccggacgctctgcgccccttcgttccttgggatcaattggaaaacgaggattccagcgtgcacatttccccccggcagaagcgagatgccaactccagcatctacaaaggcaagaagtgccgcatggagtcctgcttcgatttcaccctttgcaagaaaaacggcttcaaagtctacgtatacccacagcaaaaaggggagaaaatcgccgaaagttaccaaaacattctagcggccatcgagggctccaggttctacacctcggaccccagccaggcgtgcctctttgtcctgagtctggatactttagacagagaccagttgtcacctcagtatgtgcacaatttgagatccaaagtgcagagtctccacttgtggaacaatggtaggaatcatttaatttttaatttatattccggcacttggcctgactacaccgaggacgtggggtttgacatcggccaggcgatgctggccaaagccagcatcagtactgaaaacttccgacccaactttgatgtttctattcccctcttttctaaggatcatcccaggacaggaggggagagggggtttttgaagttcaacaccatccctcctctcaggaagtacatgctggtattcaaggggaagaggtacctgacagggataggatcagacaccaggaatgccttatatcacgtccataacggggaggacgttgtgctcctcaccacctgcaagcatggcaaagactggcaaaagcacaaggattctcgctgtgacagagacaacaccgagtatgagaagtatgattatcgggaaatgctgcacaatgccactttctgtctggttcctcgtggtcgcaggcttgggtccttcagattcctggaggctttgcaggctgcctgcgtccctgtgatgctcagcaatggatgggagttgccattctctgaagtgattaattggaaccaagctgccgtcataggcgatgagagattgttattacagattccttctacaatcaggtctattcatcaggataaaatcctagcacttagacagcagacacaattcttgtgggaggcttatttttcttcagttgagaagattgtattaactacactagagattattcaggacagaatattcaagcacatatcacgtaacagtttaatatggaacaaacatcctggaggattgttcgtactaccacagtattcatcttatctgggagattttccttactactatgctaatttaggtttaaagcccccctccaaattcactgcagtcatccatgcggtgacccccctggtctctcagtcccagccagtgttgaagcttctcgtggctgcagccaagtcccagtactgtgcccagatcatagttctatggaattgtgacaagcccctaccagccaaacaccgctggcctgccactgctgtgcctgtcgtcgtcattgaaggagagagcaaggttatgagcagccgttttctgccctacgacaacatcatcacagacgccgtgctcagccttgacgaggacacggtgctttcaacaacagaggtggatttcgccttcacagtgtggcagagcttccctgagaggattgtggggtaccccgcgcgcagccacttctgggataactctaaggagcggtggggatacacatcaaagtggacgaacgactactccatggtgttgacaggagctgctatttaccacaaatattatcactacctatactcccattacctgccagccagcctgaagaacatggtggaccaattggccaattgtgaggacattctcatgaacttcctggtgtctgctgtgacaaaattgcctccaatcaaagtgacccagaagaagcagtataaggagacaatgatgggacagacttctcgggcttcccgttgggctgaccctgaccactttgcccagcgacagagctgcatgaatacgtttgccagctggtttggctacatgccgctgatccactctcagatgaggctcgaccccgtcctctttaaagaccaggtctctattttgaggaagaaataccgagacattgagcgactttgaggaatccggctgagtgggggaggggaagcaagaagggatgggggtcaagctgctctctcttcccagtgcagatccactcatcagcagagccagattgtgccaactatccaaaaacttagatgagcagaatgacaaaaaaaaaaaggccaatgagaactcaactcctggctcctgggactgcaccagactgctccaaactcacctcactggcttctgtgtcccaagactaggttgtgtacagtttaattatggaacattaaataattatttttgaaatgattgctatgcaggtttaaacttttttaatgatcaaaactattaaaaaccagagttctttgtttaatcaaaaaaaaaaaaaaaaa

SEQ ID NO: 24 - DNA, 972 bp, Homo sapiens
tctagactcaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagatgttctaga

SEQ ID NO: 25 - DNA, 1391 bp, Homo sapiens
tgcaccccgagcatccgccccgggtggcacgtccccgagcccaccaggccggccccgtctccccatccgtctagtccgctcgcggtgccatgccattcctcgggcaggactggcggtcccccgggcagaactgggtgaagacggccgacggctggaagcgcttcctggatgagaagagcggcagtttcgtgagcgacctcagcagttactgcaacaaggaggtatacaataaggagaatcttttcaacagcctgaactatgatgttgcagccaagaagagaaagaaggacatgctgaatagcaaaaccaaaactcagtatttccaccaagaaaaatggatctatgttcacaaaggaagtactaaagagcgccatggatattgcaccctgggggaagctttcaacagactggacttctcaactgccattctggattccagaagatttaactacgtggtccggctgttggagctgatagcaaagtcacagctcacatccctgagtggcatcgcccaaaagaacttcatgaatattttggaaaaagtggtactgaaagtccttgaagaccagcaaaacattagactaataagggaactactccagaccctctacacatccttatgtacactggtccaaagagtcggcaagtctgtgctggtcgggaacattaacatgtgggtgtatcggatggagacgattctccactggcagcagcagctgaacaacattcagatcaccaggcctgccttcaaaggcctcaccttcactgacctgcctttgtgcctacaactgaacatcatgcagaggctgagcgacgggcgggacctggtcagcctgggccaggctgcccccgacctgcacgtgctcagcgaagaccggctgctgtggaagaaactctgccagtaccacttctccgagcggcagatccgcaaacgattaattctgtcagacaaagggcagctggattggaagaagatgtatttcaaacttgtccgatgttacccaaggaaagagcagtatggagatacccttcagctctgcaaacactgtcacatcctttcctggaagggcactgaccatccgtgcactgccaataacccagagagctgctccgtttcactttcaccccaggactttatcaacttgttcaagttctgaatcccagcacatgacaacacttcagaagggtccccctgctgactggagagctgggaatatggcatttggacacttcatttgtaaatagtgtacattttaaacattggctcgaaacttcagagataagtcatggagaggacattggaggggagaaatgcagttgctgactgggaatttaagaatgtgaacttctcactagaattggtatggaaaagcaaaatactgtaaataaac

SEQ ID NO: 26 - DNA, 4654 bp, Homo sapiens
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaataaaaaaaaaaaaaaa

SEQ ID NO: 27 - DNA, 1398 bp, Homo sapiens
ggagagcggggccctttgtcctccagtggctggtaggcagtggctgggaggcagcggcccaattagtgtcgtgcggcccgtggcgaggcgaggtccggggagcgagcgagcaagcaaggcgggaggggtggccggagctgcggcggctggcacaggaggaggagcccgggcgggcgaggggcggccggagagcgccagggcctgagctgccggagcggcgcctgtgagtgagtgcagaaagcaggcgcccgcgcgctagccgtggcaggagcagcccgcacgccgcgctctctccctgggcgacctgcagtttgcaatatgactttggaggaattctcggctggagagcagaagaccgaaaggatggataaggtgggggatgccctggaggaagtgctcagcaaagccctgagtcagcgcacgatcactgtcggggtgtacgaagcggccaagctgctcaacgtcgaccccgataacgtggtgttgtgcctgctggcggcggacgaggacgacgacagagatgtggctctgcagatccacttcaccctgatccaggcgttttgctgcgagaacgacatcaacatcctgcgcgtcagcaacccgggccggctggcggagctcctgctcttggagaccgacgctggccccgcggcgagcgagggcgccgagcagcccccggacctgcactgcgtgctggtgacgaatccacattcatctcaatggaaggatcctgccttaagtcaacttatttgtttttgccgggaaagtcgctacatggatcaatgggttccagtgattaatctccctgaacggtgatggcatctgaatgaaaataactgaaccaaattgcactgaagtttttgaaatacctttgtagttactcaagcagttactccctacactgatgcaaggattacagaaactgatgccaaggggctgagtgagttcaactacatgttctgggggcccggagatagatgactttgcagatggaaagaggtgaaaatgaagaaggaagctgtgttgaaacagaaaaataagtcaaaaggaacaaaaattacaaagaaccatgcaggaaggaaaactatgtattaatttagaatggttgagttacattaaaataaaccaaatatgttaaagtttaagtgtgcagccatagtttgggtatttttggtttatatgccctcaagtaaaagaaaagccgaaagggttaatcatatttgaaaaccatattttattgtattttgatgagatattaaattctcaaagttttattataaattctactaagttattttatgacatgaaaagttatttatgctataaattttttgaaacacaatacctacaataaactggtatgaataattgcatcatttcaaaaaaaaaaaaaaaaaa

SEQ ID NO: 28 - DNA, 11242 bp, Homo sapiens
ttttttttttttttttttgagaaaggggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctgatccttggatcctgaatctgtgcaaacagtaacgtgtgcgcacgcgcagcggggtggggggggagagagagttttaacaatccattcacaagcctcctgtacctcagtggatcttcagaactgcccttgctgcccgcgggagacagcttctctgcagtaaaacacatttgggatgttccttttttcaatatgcaagcagctttttattccctgcccaaacccttaactgacatgggcctttaagaaccttaatgacaacacttaatagcaacagagcacttgagaaccagtctcctcactctgtccctgtccttccctgttctccctttctctctcctctctgcttcataacggaaaaataattgccacaagtccagctgggaagccctttttatcagtttgaggaagtggctgtccctgtggccccatccaaccactgtacacacccgcctgacaccgtgggtcattacaaaaaaacacgtggagatggaaatttttacctttatctttcacctttctagggacatgaaatttacaaagggccatcgttcatccaaggctgttaccattttaacgctgcctaattttgccaaaatcctgaactttctccctcatcggcccggcgctgattcctcgtgtccggaggcatgggtgagcatggcagctggttgctccatttgagagacacgctggcgacacactccgtccatccgactgcccctgctgtgctgctcaaggccacaggcacacaggtctcattgcttctgactagattattatttgggggaactggacacaataggtctttctctcagtgaaggtggggagaagctgaaccggcttccctgccctgcctccccagccccctgcccaacccccaagaatctggtggccatgggccccgaagcagcctggcggacaggcttggagtcaaggggccccatgcctgcttctctcccagccccagctcccccgcccgcccccaaggacacagatgggaaggggtttccagggactcagccccactgttgatgcaggtttgcaaggaaagaaattcaaacaccacaacagcagtaagaagaaaagcagtcaatggattcaagcattctaagctttgttgacattttctctgttcctaggacttcttcatgggtcttacagttctatgttagaccatgaaacatttgcatacacatcgtctttaatgtcacttttataacttttttacggttcagatattcatctatacgtctgtacagaaaaaaaaaagctgctattttttttgttcttgatctttgtggatttaatctatgaaaaccttcaggtccaccctctcccctttctgctcactccaagaaacttcttatgctttgtactagagtgcgtgactttcttcctcttttcccggtaatggatacttctatcacataatttgccatgaactgttggatgcctttttataaatacatcccccatccctgctcccacctgcccctttagttgttttctaacccgtaggctctctgggcacgaggcagaaagcaggccgggcacccatcctgagagggccgcgctcctctccccagcctgccctcacagcattggagcctgttacagtgcaagacatgatacaaactcaggtcagaaaaacaaaggttaaatatttcacacgtctttgttcagtgtttccactcaccgtggttgagaagcctcaccctctctttcccttgcctttgcttaggttgtgacacacatatatatatatttttttaattcttgggtacaacagcagtgttaaccgcagacactaggcatttggattactatttttcttaatggctatttaatccttccatcccacgaaaaacagctgctgagtccaagggagcagcagagcgtggtccggcagggcctgttgtggccctcgccacccccctcaccggaccgactgacctgtctttggaaccagaacatcccaagggaactccttcgcactggcgttgagtgggaccccgggatccaggctggcccagggcggcaccctcagggctgtgcccgctggagtgctaggtggaggcagcacagacgccacggtggcccaagagcccctttgcttcttgctgggggaccagggctgtggtgctggcccactttccctcggccaggaatccaggtccttggggcccaggggtcttgtcttgtttcatttttagcacttctcaccagagagatgacagcacaagagttgcttctgggatagaaatgtttaggagtaagaacaaagctgggatacggtgattgctagttgtgactgaagattcaacacagaaaagaaagtttatacggcttttttgctggtcagcagtttgtcccactgctttctctagtctctatcccatagcgtgttccctttaaaaaaaaaaaaaaggtattatatgtaggagttttcttttaatttattttgtgataaattaccagtttcaatcactgtagaaaagccccattatgaatttaaatttcaaggaaagggtgtgtgtgtgtgtatgtgtggggtgtgtgtgtgtgagagtgatgggacagttcttgattttttgggttttttttcccccaaacatttatctacctcactcttattttttatatgtgtatatagacaaaagaatacatctcacctttctcagcacctgacaataggccgttgatactggtaacctcatccacgccacaggcgccacacccaggtgatgcagggggaagccaggctgtattccggggtcaaagcaacactaactcacctctctgctcatttcagacagcttgcctttttctgagatgtcctgttttgtgttgctttttttgttttgttttctatcttggtttccaccaaggtgttagatttctcctcctcctagccaggtggccctgtgaggccaacgagggcaccagagcacacctgggggagccaccaggctgtccctggctggttgtctttggaacaaactgcttctgtgcagatggaatgaccaacacatttcgtccttaagagagcagtggttcctcaggttctgaggagaggaaggtgtccaggcagcaccatctctgtgcgaatccccagggtaaaggcgtggggcattgggtttgctccccttgctgctgctccatccctgcaggaggctcgcgctgaggcaggaccgtgcggccatggctgctgcattcattgagcacaaaggtgcagctgcagcagcagctggagagcaagagtcacccagcctgtgcgccagaatgcagaggctcctgacctcacagccagtccctgatagaacacacgcaggagcagagtcccctccccctccaggctgccctctcaacttctccctcacctccttccctaggggtagacagagatgtaccaaaccttccggctggaaagcccagtggccggcgccgaggctcgtggcgtcacgccccccccgccagggctgtacctccgtctccctggtcctgctgctcacaggacagacggctcgctcccctcttccagcagctgctcttacaggcactgatgatttcgctgggaagtgtggcgggcagctttgcctaagcgtggatggctcctcggcaattccagcctaagtgaaggcgctcaggagcctcctgctggaacgcgacccatctctcccaggaccccggggatcttaaggtcattgagaaatactgttggatcagggttttgttcttccacactgtaggtgaccccttggaataacggcctctcctctcgtgcacatacctaccggtttccacaactggatttctacagatcattcagctggttataagggttttgtttaaactgtccgagttactgatgtcattttgtttttgttttatgtaggtagcttttaagtagaaaacactaacagtgtagtgcccatcatagcaaatgcttcagaaacacctcaataaaagagaaaacttggcttgtgtgatggtgcagtcactttactggaccaacccacccaccttgactataccaaggcatcatctatccacagttctagcctaacttcatgctgatttctctgcctcttgatttttctctgtgtgttccaaataatcttaagctgagttgtggcattttccatgcaacctccttctgccagcagctcacactgcttgaagtcatatgaaccactgaggcacatcatggaattgatgtgagcattaagacgttctcccacacagcccttccctgaggcagcaggagctggtgtgtactggagacactgttgaacttgatcaagacccagaccaccccaggtctccttcgtgggatgtcatgacgtttgacatacctttggaacgagcctcctccttggaagatggaagaccgtgttcgtggccgacctggcctctcctggcctgtttcttaagatgcggagtcacatttcaatggtacgaaaagtggcttcgtaaaatagaagagcagtcactgtggaactaccaaatggcgagatgctcggtgcacattggggtgctttgggataaaagatttatgagccaactattctctggcaccagattctaggccagtttgttccactgaagcttttcccacagcagtccacctctgcaggctggcagccgaatggcttgccagtggctctgtggcaagatcacactgagatcgatgggtgagaaggctaggatgcttgtctagtgttcttagctgtcacgttggctccttccagggtggccagacggtgttggccactcccttctaaaacacaggcgccctcctggtgacagtgacccgccgtggtatgccttggcccattccagcagtcccagttatgcatttcaagtttggggtttgttcttttcgttaatgttcctctgtgttgtcagctgtcttcatttcctgggctaagcagcattgggagatgtggaccagagatccactccttaagaaccagtggcgaaagacactttctttcttcactctgaagtagctggtggtacaaatgagaacttcaagagaggatgttatttagactgaacctctgttgccagagatgctgaagatacagaccttggacaggtcagagggtttcatttttggccttcatcttagatgactggttgcgtcatttggagaagtgagtgctccttgatggtggaatgaccgggtggtgggtacagaaccattgtcacagggatcctggcacagagaagagttacgagcagcagggtgcagggcttggaaggaatgtgggcaaggttttgaacttgattgttcttgaagctatcagaccacatcgaggctcagcagtcatccgtgggcatttggtttcaacaaagaaacctaacatcctactctggaaactgatctcggagttaaggcgaattgttcaagaacacaaactacatcgcactcgtcagttgtcagttctggggcatgactttagcgttttgtttctgcgagaacataacgatcactcatttttatgtcccacgtgtgtgtgtccgcatctttctggtcaacattgttttaactagtcactcattagcgttttcaatagggctcttaagtccagtagattacgggtagtcagttgacgaagatctggtttacaagaactaattaaatgtttcattgcatttttgtaagaacagaataattttataaaatgtttgtagtttataattgccgaaaataatttaaagacactttttttttctctgtgtgtgcaaatgtgtgtttgtgatccattttttttttttttttttaggacacctgtttactagctagctttacaatatgccaaaaaaggatttctccctgaccccatccgtggttcaccctcttttccccccatgctttttgccctagtttataacaaaggaatgatgatgatttaaaaagtagttctgtatcttcagtatcttggtcttccagaaccctctggttgggaaggggatcattttttactggtcatttccctttggagtgtagctactttaacagatggaaagaacctcattggccatggaaacagccgaggtgttggagcccagcagtgcatggcaccgttcggcatctggcttgattggtctggctgccgtcattgtcagcacagtgccatggacatgggaagacttgactgcacagccaatggttttcatgatgattacagcatacacagtgatcacataaacgatgacagctatggggcacacaggccatttgcttacatgcctcgtatcatgactgattactgctttgttagaacacagaagagaccctattttatttaaggcagaaccccgaagatacgtatttccaatacagaaaagaatttttaataaaaactataacatacacaaaaattggttttaaagttgactccacttcctctaactccagtggattgttggccatgtctccccaactccacaatatctctatcatgggaaacacctggggtttttgcgctacataggagaaagatctggaaactatttgggttttgttttcaacttttcatttggatgtttggcgttgcacacacacatccaccggtggaagagacgcccggtgaaaacacctgtctgctttctaagccagtgaggttgaggtgagaggtttgccagagtttgtctacctctgggtatccctttgtctgggataaaaaaaatcaaaccagaaggcgggatggaatggatgcaccgcaaataatgcattttctgagttttcttgttaaaaaaaaatttttttaagtaagaaaaaaaaaggtaataacatggccaatttgttacataaaatgactttctgtgtataaattattcctaaaaaatcctgtttatataaaaaatcagtagatgaaaaaaatttcaaaatgtttttgtatattctgttgtaagaatttattcctgttattgcgatatactctggattctttacataatggaaaaaagaaactgtctattttgaatggctgaagctaaggcaacgttagtttctcttactctgcttttttctagtaaagtactacatggtttaagttaaataaaataattctgtatgca

SEQ ID NO: 29 - DNA, 1660 bp, Homo sapiens
ggtgcactagcaaaacaaacttattttgaacactcagctcctagcgtgcggcgctgccaatcattaacctcctggtgcaagtggcgcggcctgtgccctttataaggtgcgcgctgtgtccagcgagcatcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtctataatggaaacatttacaataaatattctgcatggaaatactgttaaaaaaaaaaa

SEQ ID NO: 30 - DNA, 2638 bp, Homo sapiens
agatgcgagcactgcggctgggcgctgaggatcagccgcttcctgcctggattccacagcttcgcgccgtgtactgtcgccccatccctgcgcgcccagcctgccaagcagcgtgccccggttgcaggcgtcatgcagcgggcgcgacccacgctctgggccgctgcgctgactctgctggtgctgctccgcgggccgccggtggcgcgggctggcgcgagctcggcgggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggtgagccgcccgcgccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtagacgcctgccgcaaggttaatgtggagctcaaatatgccttattttgcacaaaagactgccaaggacatgaccagcagctggctacagcctcgatttatatttctgtttgtggtgaactgattttttttaaaccaaagtttagaaagaggtttttgaaatgcctatggtttctttgaatggtaaacttgagcatcttttcactttccagtagtcagcaaagagcagtttgaattttcttgtcgcttcctatcaaaatattcagagactcgagcacagcacccagacttcatgcgcccgtggaatgctcaccacatgttggtcgaagcggccgaccactgactttgtgacttaggcggctgtgttgcctatgtagagaacacgcttcacccccactccccgtacagtgcgcacaggctttatcgagaataggaaaacctttaaaccccggtcatccggacatcccaacgcatgctcctggagctcacagccttctgtggtgtcatttctgaaacaagggcgtggatccctcaaccaagaagaatgtttatgtcttcaagtgacctgtactgcttggggactattggagaaaataaggtggagtcctacttgtttaaaaaatatgtatctaagaatgttctagggcactctgggaacctataaaggcaggtatttcgggccctcctcttcaggaatcttcctgaagacatggcccagtcgaaggcccaggatggcttttgctgcggccccgtggggtaggagggacagagagacagggagagtcagcctccacattcagaggcatcacaagtaatggcacaattcttcggatgactgcagaaaatagtgttttgtagttcaacaactcaagacgaagcttatttctgaggataagctctttaaaggcaaagctttattttcatctctcatcttttgtcctccttagcacaatgtaaaaaagaatagtaatatcagaacaggaaggaggaatggcttgctggggagcccatccaggacactgggagcacatagagattcacccatgtttgttgaacttagagtcattctcatgcttttctttataattcacacatatatgcagagaagatatgttcttgttaacattgtatacaacatagccccaaatatagtaagatctatactagataatcctagatgaaatgttagagatgctatatgatacaactgtggccatgactgaggaaaggagctcacgcccagagactgggctgctctcccggaggccaaacccaagaaggtctggcaaagtcaggctcagggagactctgccctgctgcagacctcggtgtggacacacgctgcatagagctctccttgaaaacagaggggtctcaagacattctgcctacctattagcttttctttatttttttaactttttggggggaaaagtatttttgagaagtttgtcttgcaatgtatttataaatagtaaataaagtttttaccattaaaaaaatatctttccctttgttattgaccatctctgggctttgtatcactaattattttattttattatataataattattttattataataaaatcctgaaaggggaaaataaaaaaaa

SEQ ID NO: 31 - DNA, 4723 bp, Homo sapiens
ggggggctgcgcggccgggtcggtgcgcacacgagaaggacgcgcggcccccagcgctcttgggggccgcctcggagcatgacccccgcgggccagcgccgcgcgcctgatccgaggagaccccgcgctcccgcagccatgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataatcacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccaatacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccaccactcaggggaaactcttcttccactataaccccaaactctgcttgtcagaaatccacaagatggaagaagtttcaggaaccaaggggcgccaggagagaaacgacattgccctgaagaccaatggggacaaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgatgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccagaaaaacctcttcaggcactggtgccgaggaccctaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccagtggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattcttttttttttttttttttttttttttttgctggtgtctgagcttcagtataaaagacaaaacttcctgtttgtggaacaaaatttcgaaagaaaaaaccaaa

SEQ ID NO: 32 - DNA, 3017 bp, Homo sapiens
ggatccctgagcgtcacgccgctgttgtggagcgcgtgttgacaacgtcgccggggagacgggcgggggcggggcccgggagagggggaggcgcggccctggcggcgcgcgaggggccgggctgtcagcgcaaggcccaggccgccgcagtggccacggccgctgccgcccgccggcttatataccgcggctaaatttaggctgcgcccggagctcgtccccatccgggacgcgtttccgccgccgccgctttggcccggcccccgcgcgcgccgcgcctataaggcttgggcgggcccggccgcggcccacagagccgtccccgcccgcccgcgccccgaccagcccggcctcgggcagccactcaccggtgtccccgtccgcgtccttcctccccgggtcccggccatggcgctgagtgaacccatcctgccgtccttttccactttcgccagcccgtgccgcgagcgcggcctgcaggaggtgagggcggcggggacggcggggcgaccgggaccgtgggcggcgggctcggggtagtagaacgtgggctgcggggtgacaggacgcgaaggcggggactgcagactcaggagaggaggatgcgggccacggggatcgcggacttagggtggtaaaaggcaagcagcgccccccgagccccgccgcccgctcacgcccattgccctgtcgcccgcagcgctggccgcgcgccgaacccgagtccggcggcaccgacgacgacctcaacagcgtgctggacttcatcctgtccatggggctggatggcctgggcgccgaggccgccccggagccgccgccgccgcccccgccgcctgcgttctattaccccgaacccggcgcgcccccgccctacagcgcccccgcgggtggcctggtgtctgagctgctgcgacccgagctggatgcgccgccggggcccgcactgcacggccgctttctgctggcgccgcccggccgcctggtcaaggccgagccccctgaagcggacggcggcggcggctacggctgcgcccccgggctgacccgtggaccgcgcggcctcaagcgcgagggcgccccaggcccggcggcttcgtgcatgcgaggtcccgggggccgccccccgccgccgcccgacacaccgccgctcagccccgacggccccgcgcgcctgcccgcgcccggtccgcgcgcctccttcccgccgcctttcggtggccctggtttcggcgcgcccgggcccggcctgcattacgcgccgcctgcgcccccagccttcggtcttttcgacgacgcggccgccgccgcggcagccctgggcctggcgccccccgccgcccgcggtctcctcacgccgcctgcgtccccgctggagctgctggaggccaagccaaagcgcggccgccgctcttggccccgcaaacgcaccgccactcacacctgcagctacgcgggctgcggcaagacctacaccaagagttcgcatctgaaggcgcatctgcgcacgcacacaggtgggcggcacgcacgagccaggagcgcaggcggggggacgcgggaggagaggtcggattcccagcgcgcgccagaaaatgaatttaggacctcccttggggcgtggctcagggggatctggcagtggtgcacgcttaggactccccaggagcgtggctcgggaggttggttgggggggcacacaggaacactccctaaggaagtgtgatccgagaggttggggtgggggcttgcacgcttaggacgaggggggcctccggaggttgggaagagcacttagaaaacctcctggaggcgtggctagggagacagtctcagaaagttggggagggggagcaggtttaggagccgctgggcacttggctcagaatccccggggctgaggctcaggtagttggggagtaggtgcgcgtttaggaaccccggggagatgctgcgtctcaggaagttggggagggcgctcaggcttgggactcctctggggacaaggctcaggaccttggggagggagtgttcgctgggaaaccttgagagattccgtgtcttagaatgctggagagaggtgcatgcttaggaccgtcggggagcgtggctgacaacagtggggagtggaccttgcgctcctccgaccccctgggggtgaggatccggattgtggggggagttggggatgtagggcaaggatccctcaggggcgcaacactaccgcggggagcgcgtcaaggccctggttagggataggttgcgctcgccggggtagccatacgtgccctgtcctgggaggggaactgacgcttactctcgccccctccctgcaggtgagaagccctaccactgcaactgggacggctgcggctggaagtttgcgcgctcagacgagctcacgcgccactaccgaaagcacacgggccaccggccattccagtgccatctgtgcgatcgtgccttctcgcgctccgatcacctggcgctgcacatgaaacggcacatgtagccgggacgcccccgcccacctgcgcgcggccgtggcgggtcccacgcgccgggcgcggccccctcccaaactgtgactggtatttattggacccagagaaccgggccgggcacagcgtggctacagagggtctccctcgatgacgacgacgacgacgccaccaccccagcccccgtctgtgactgaaggcccggtgggaaaagaccacgatcctccttgacgagttttgtttttcaaaatggtgcaataatttaagtggcatcttctctcccaccgggtctacactagaggatcgaggcttgtgatgccttgtaagaaataagggccttaatttgtactgtctgcggcattttttataatattgtatatagtgactgacaaatattgtattactgtacatagagagacaggtgggcatttttgggctacctggttcgtttttataagattttgctgggttggtttttttttttattaaaaagttttgcatcttttaaaaaaaaaa

SEQ ID NO: 33 - DNA, 2949 bp, Homo sapiens
agtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaaaaaaaaaaaaaaaaaa

SEQ ID NO: 34 - DNA, 2073 bp, Homo sapiens
agatgccacgccccatagctccaccagtcaccgcggcacagtggcccttaagcgaggagcggcggcgcccgcagcaatcacagcagtgccgacgtcgtgggtgtttggtgtgaggctgcgagccgccgcgagttctcacggtcccgccggcgccaccaccgcggtcactcaccgccgccgccgccaccactgccaccacggtcgcctgccacaggtgtctgcaattgaactccaaggtgcagaatggtttggaaagtagctgtattcctcagtgtggccctgggcattggtgccgttcctatagatgatcctgaagatggaggcaagcactgggtggtgatcgtggcaggttcaaatggctggtataattataggcaccaggcagacgcgtgccatgcctaccagatcattcaccgcaatgggattcctgacgaacagatcgttgtgatgatgtacgatgacattgcttactctgaagacaatcccactccaggaattgtgatcaacaggcccaatggcacagatgtctatcagggagtcccgaaggactacactggagaggatgttaccccacaaaatttccttgctgtgttgagaggcgatgcagaagcagtgaagggcataggatccggcaaagtcctgaagagtggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagattcggacgtggaagatctgactaaagagaccctgcacaagcagtaccacctggtaaaatcgcacaccaacaccagccacgtcatgcagtatggaaacaaaacaatctccaccatgaaagtgatgcagtttcagggtatgaaacgcaaagccagttctcccgtccccctacctccagtcacacaccttgacctcacccccagccctgatgtgcctctcaccatcatgaaaaggaaactgatgaacaccaatgatctggaggagtccaggcagctcacggaggagatccagcggcatctggatgccaggcacctcattgagaagtcagtgcgtaagatcgtctccttgctggcagcgtccgaggctgaggtggagcagctcctgtccgagagagccccgctcacggggcacagctgctacccagaggccctgctgcacttccggacccactgcttcaactggcactcccccacgtacgagtatgcgttgagacatttgtacgtgctggtcaacctttgtgagaagccgtatccgcttcacaggataaaattgtccatggaccacgtgtgccttggtcactactgaagagctgcctcctggaagcttttccaagtgtgagcgccccaccgactgtgtgctgatcagagactggagaggtggagtgagaagtctccgctgctcgggccctcctggggagcccccgctccagggctcgctccaggaccttcttcacaagatgacttgctcgctgttacctgcttccccagtcttttctgaaaaactacaaattagggtgggaaaagctctgtattgagaagggtcatatttgctttctaggaggtttgttgttttgcctgttagttttgaggagcaggaagctcatgggggcttctgtagcccctctcaaaaggagtctttattctgagaatttgaagctgaaacctctttaaatcttcagaatgattttattgaagagggccgcaagccccaaatggaaaactgtttttagaaaatatgatgatttttgattgcttttgtatttaattctgcaggtgttcaagtcttaaaaaataaagatttataacagaacccaaataaaaaaaaaaaaaaaaa

SEQ ID NO: 35 - DNA, 3470 bp, Homo sapiens
atacacacagactcacagcgagaccgacacacactcccatacactcacacacacaactgcaggcagcgaggctcgggaagtcaggccggcttttcgccccggcgccttctctgctccagccggccgggtctccctgggggcccggagctcggccgggccgcgcagccccgttagaggacgagctcggcggacccccgctcctccatgggcaaacgcgggcggccgcgcaaggaggcgcgctgcgagggcgcggggctggcccccgccgcgcccccggctgtgccccccgccgtggccgcgccccagcccccggccctgcccgaggaccccgctggggccaagcccaggtgccccttctcagacattttcaacaccagcgagaactcgatggagaagcacatcaacacttttctgcagaacgtgcagattctgctcgaggccgccagctacctggagcagatcgagaaagaaaacaaaaagtgtgaacatggctacgcctcttcattcccgtccatgccgagcccccgactgcagcattcaaagcccccacggaggttgagccgggcacagaaacacagcagcgggagcagcaacaccagcactgccaacagatctacacacaatgagctggaaaagaatcgacgagctcatctgcgcctttgtttagaacgcttaaaagttctgattccactaggaccagactgcacccggcacacaacacttggtttgctcaacaaagccaaagcacacatcaagaaacttgaagaagctgaaagaaaaagccagcaccagctcgagaatttggaacgagaacagagatttttaaagtggcgactggaacagctgcagggtcctcaggagatggaacgaatacgaatggacagcattggatcaactatttcttcagatcgttctgattcagagcgagaggagattgaagtggatgttgaaagcacagagttctcccatggagaagtggacaatataagtaccaccagcatcagtgacattgatgaccacagcagcctgccgagtattgggagtgacgagggttactccagtgccagtgtcaaactttcattcacttcatagaacccagcatgacataacagtgcagggcaaaatattcactgggccaattcaatacaaacaatctcttaaattgggttcatgatgcagtctcctctttaaaacaaaacaaaacaaaacaaaactatacttgaacaaaagggtcagaggacctgtatttaagcaaatacttagcaaaaagtggggcagagcctcccaaggagaacaaatattcagaatattcatattggaaaaatcacaatttttaatggcagcagaaaacttgtgtgaaattttcttgatttgagttgattgagaagaggacattggagatgccatcctctttctcttttctagtttgctcatactacattgagtagacacatttaaggatggggttatgaacccttcctgagctttatggtcctaaaagcaaaataaaaactattcgaatgaaaagacaagaaaatcaggtattaatcttggatagctaataatgagctattaaaactcagcctgggacagtttatcatgaagcctgtggatgatcaatcctttattattattttttttttttgaaaaaagctcatttcatgctctgcaaaaggagagactcccatgaagccttttgaaagggatcatcatgcagctcaactttctgttggattccatgctaagcaagctaaccttatcctgcattgttagcactaggcacccagctgccacctctccatcctgctgcccttaggccacatgggagcagtccatgcatgacagcctctatcctacaaggcctatgagtatggattgggggggccaaaaggaaaaagctccatgtgcctctttgtctgcgtgggtcagaagagttgtgcacgcagattagcaggccaaggtctgagccacagcagcatttttatttcagattttgataactgtttatatgtgttgaaaaccaaaatgacatctttttaaagcttatccataaaaaaaaatagatgtcttttatagtggaaaaacacatggggaaaaaaatcatctattttgatgcagcatttgataatgataaaacacctcacacctcactctttatagtgcacaaaatgaatgaggtctgggctaggtagaaaaagggtcaatgctatttttgtttttagaatcattaccttttaccagcttttaaccatctgatatctatagtagacacactatcatagttaacatagtaagttcagcacttgtctcattttaatgtaaagatttgcttccattttcctacaggcagtctctctcttcctcacagtcccactgtgcaggtgctattgttactcttacgaatattttcagtaatgttattttcttctaagtgaaatttctagcctgcactttgatgtcatgtgttccctttgtctttcaaactccaaggttcccttgtggccctctcccttaccctgggaaggcctcttggagaccttacccctggctgtttggactttgtatactttaaataatttaactacccttaattacttaaaaaaaaaaaaaagctttatgattttcataacttattgctgattttaatggattgttaatttcagtcctgtagttttattttatgtttagatagggctgggcaaggaaaaagaaaataaagacaaccatatttagcagtgcagttgagttgtgtgttaatgttagactatccctttgtgagtgacactttaacagcattcactgcttctatatatagtgtaccatcttggtcatacattacgcctcaacatatacttgtgctcttcctttgcctccagaagaagtttttccttgattgtgctatgtttcagtggaagaaattctttgaagtagatgtgagtgaaaaactgcatgcctttagaagcccagtatcagaacttgctacgtttcaggtgctagggacttaatgaaaaacaggacaaaacaattcctttttgtggcccaggtaaattatttctggtttcacttataattactaatggctgagtcaagatgttgtctctgtgtttgcttactcttgatcaagtgtgagacagtttgaagactgtgctaccatacaaagtgaatgaagccagtgactaagcttctgtttgttttgttattctcatggccttcgcttgcattatttgggccttcattcagatgaacttgaggtgccattttgttgcatatgtacaggattatgggctggaaagcatttgttataaacctatagtgcacattttaactgccccctaaattacccttccctgggtttgttttccttggggtggtgtagattgtatgagtaagaagtattaattttttaaaagacaaatcaactttgaagacacaaaagttaattggaagaaataaaaactgtgaacgaagaa

SEQ ID NO: 36 - DNA, 1823 bp, Homo sapiens
gagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggatatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctgaggggatggtggccgcccacccgcccgagggatggtgcccctagggtccctcgcgcccaaaagattgaacttaaatgcccccctcccaacagcgctttaaaagcgacctctcttgaggtaggagaggcgggagaactgaagtttccgcccccgccccacagggcaaggacacagcgcggttttttccacgcagcacccttctcggagacccattgcgatggccgctccgtgttcctcggtgggccagagctgaaccttgaggggctaggttcagctttctcgcgccctcccccatgggggtgagaccctcgcagacctaagccctgccccgggatgcaccggttatttgggggggcgtgagacccagtgcactccggtcccaaatgtagcaggtgtaaccgtaacccacccccaacccgtttcccggttcaggaccactttttgtaatacttttgtaatctattcctgtaaataagagttgctttgccagagcaggagcccctggggctgtatttatctctgaggcatggtgtgtggtgctacagggaatttgtacgtttataccgcaggcgggcgagccgcgggcgctcgctcaggtgatcaaaataaaggcgctaatttataaaaaaaaaaaaaaaaaa

SEQ ID NO: 37 - DNA, 4345 bp, Homo sapiens
actgaaactaggggcaaggagacgaagagaacatgaaagttaaactttaagatgaagaacaaagctgaacatactgatgcattggatctttggagaggatctcagaactcattgtacttaatttacaggctaaaaccttagaagaggaatttattatatcctacacaagactccagggaagcacatggccttggactgaaggctggcatctggaagctgtcagccaccagcaccttctgcagcaggaaaaggccagggctctgctggagcaggcagcagagtggacgcacagtaacatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctggaacagccagctggtgcgctacgcgggctaccggcagcaggatggctctgtgcggggggacccagccaacgtggagatcaccgagctctgcattcagcacggctggaccccaggaaacggtcgcttcgacgtgctgcccctgctgctgcaggccccagatgatcccccagaactcttccttctgccccccgagctggtccttgaggtgcccctggagcaccccacgctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggatgtggctgtctgcatggacctggatacccggaccacctcgtccctgtggaaagacaaggcagcagtggaaatcaacgtggccgtgctgcacagttaccagctagccaaagtcaccatcgtggaccaccacgccgccacggcctctttcatgaagcacctggagaatgagcagaaggccagggggggctgccctgcagactgggcctggatcgtgccccccatctcgggcagcctcactcctgttttccatcaggagatggtcaactatttcctgtccccggccttccgctaccagccagacccctggaaggggagtgccgccaagggcaccggcatcaccaggaagaagacctttaaagaagtggccaacgccgtgaagatctccgcctcgctcatgggcacggtgatggcgaagcgagtgaaggcgacaatcctgtatggctccgagaccggccgggcccagagctacgcacagcagctggggagactcttccggaaggcttttgatccccgggtcctgtgtatggatgagtatgacgtggtgtccctcgaacacgagacgctggtgctggtggtaaccagcacatttgggaatggggatcccccggagaatggagagagctttgcagctgccctgatggagatgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgggctcggctcccgggcatacccccacttctgcgcctttgctcgtgccgtggacacacggctggaggaactgggcggggagcggctgctgcagctgggccagggcgacgagctgtgcggccaggaggaggccttccgaggctgggcccaggctgccttccaggccgcctgtgagaccttctgtgtgggagaggatgccaaggccgccgcccgagacatcttcagccccaaacggagctggaagcgccagaggtaccggctgagcgcccaggccgagggcctgcagttgctgccaggtctgatccacgtgcacaggcggaagatgttccaggctacaatccgctcagtggaaaacctgcaaagcagcaagtccacgagggccaccatcctggtgcgcctggacaccggaggccaggaggggctgcagtaccagccgggggaccacataggtgtctgcccgcccaaccggcccggccttgtggaggcgctgctgagccgcgtggaggacccgccggcgcccactgagcccgtggcagtagagcagctggagaagggcagccctggtggccctccccccggctgggtgcgggacccccggctgcccccgtgcacgctgcgccaggctctcaccttcttcctggacatcacctccccacccagccctcagctcttgcggctgctcagcaccttggcagaagagcccagggaacagcaggagctggaggccctcagccaggatccccgacgctacgaggagtggaagtggttccgctgccccacgctgctggaggtgctggagcagttcccgtcggtggcgctgcctgccccactgctcctcacccagctgcctctgctccagccccggtactactcagtcagctcggcacccagcacccacccaggagagatccacctcactgtagctgtgctggcatacaggactcaggatgggctgggccccctgcactatggagtctgctccacgtggctaagccagctcaagcccggagaccctgtgccctgcttcatccggggggctccctccttccggctgccacccgatcccagcttgccctgcatcctggtgggtccaggcactggcattgcccccttccggggattctggcaggagcggctgcatgacattgagagcaaagggctgcagcccactcccatgactttggtgttcggctgccgatgctcccaacttgaccatctctaccgcgacgaggtgcagaacgcccagcagcgcggggtgtttggccgagtcctcaccgccttctcccgggaacctgacaaccccaagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagcccctgagagccgcctggctttcccttccagttccgggagagcggctgcccgactcaggtccgcccgaccaggatcagccccgctcctcccctcttgaggtggtgccttctcacatctgtccagaggctgcaaggattcagcattattcctccaggaaggagcaaaacgcctcttttccctctctaggcctgttgcctcgggcctgggtccgccttaatctggaaggcccctcccagcagcggtaccccagggcctactgccacccgcttcctgtttcttagtcgaatgttagattcctcttgcctctctcaggagtatcttacctgtaaagtctaatctctaaatcaagtatttattattgaagatttaccataagggactgtgccagatgttaggagaactactaaagtgcctaccccagctcatgtggattacaaaaaaaa

SEQ ID NO: 38 - DNA, 2692 bp, Homo sapiens
tttaaagctgggaggttctgccaccaagcacggccttcccactgggaacacaaacttgctggcgggaagagcccggaaagaaacctgtggatctcccttcgagatcatccaaagagaagaaaggtgacctcacattcgtgccccttagcagcactctgcagaaatgcctcctcagctgcaaaacggcctgaacctctcggccaaagttgtccagggaagcctggacagcctaccccaggcagtgagggagtttctcgagaataacgctgagctgtgtcagcctgatcacatccacatctgtgacggctctgaggaggagaatgggcggcttctgggccagatggaggaagagggcatcctcaggcggctgaagaagtatgacaactgctggttggctctcactgaccccagggatgtggccaggatcgaaagcaagacggttatcgtcacccaagagcaaagagacacagtgcccatccccaaaacaggcctcagccagctcggtcgctggatgtcagaggaggattttgagaaagcgttcaatgccaggttcccagggtgcatgaaaggtcgcaccatgtacgtcatcccattcagcatggggccgctgggctcgcctctgtcaaagatcggcatcgagctgacggattcaccctacgtggtggccagcatgcggatcatgacgcggatgggcacgcccgtcctggaagcagtgggcgatggggagtttgtcaaatgcctccattctgtggggtgccctctgcctttacaaaagcctttggtcaacaactggccctgcaacccggagctgacgctcatcgcccacctgcctgaccgcagagagatcatctcctttggcagtgggtacggcgggaactcgctgctcgggaagaagtgctttgctctcaggatggccagccggctggccaaggaggaagggtggctggcagagcacatgctgattctgggtataaccaaccctgagggtgagaagaagtacctggcggccgcatttcccagcgcctgcgggaagaccaacctggccatgatgaaccccagcctccccgggtggaaggttgagtgcgtcggggatgacattgcctggatgaagtttgacgcacaaggtcatttaagggccatcaacccagaaaatggctttttcggtgtcgctcctgggacttcagtgaagaccaaccccaatgccatcaagaccatccagaagaacacaatctttaccaatgtggccgagaccagcgacgggggcgtttactgggaaggcattgatgagccgctagcttcaggtgtcaccatcacgtcctggaagaataaggagtggagctcagaggatggggaaccttgtgcccaccccaactcgaggttctgcacccctgccagccagtgccccatcattgatgctgcctgggagtctccggaaggtgttcccattgaaggcattatctttggaggccgtagacctgctggtgtccctctagtctatgaagctctcagctggcaacatggagtctttgtgggggcggccatgagatcagaggccacagcggctgcagaacataaaggcaaaatcatcatgcatgacccctttgccatgcggcccttctttggctacaacttcggcaaatacctggcccactggcttagcatggcccagcacccagcagccaaactgcccaagatcttccatgtcaactggttccggaaggacaaggaaggcaaattcctctggccaggctttggagagaactccagggtgctggagtggatgttcaaccggatcgatggaaaagccagcaccaagctcacgcccataggctacatccccaaggaggatgccctgaacctgaaaggcctggggcacatcaacatgatggagcttttcagcatctccaaggaattctgggagaaggaggtggaagacatcgagaagtatctggaggatcaagtcaatgccgacctcccctgtgaaatcgagagagagatccttgccttgaagcaaagaataagccagatgtaatcagggcctgagtgctttacctttaaaatcattccctttcccatccataaggtgcagtaggagcaagagagggcaagtgttcccaaattgacgccaccataataatcatcaccacaccgtgagcagatctgaaaggcacactttgatttttttaaggataagaaccacagaacactgggtagtagctaatgaaattgagaagggaaatcttagcatgcctccaaaaattcacatccaatgcatagtttgttcaaatttaaggttactcaggcattgatcttttcagtgttttttcactttagctatgtggattagctagaatgcacaccaaaaaaatacttgagctgtatatatatatgtgtgtgtgtgtgtgtgtgtgtgtgtgtgtgtgtgcatgtatgtgcacatgtgtctgtgtggtatatttgtgtatgtgtatttgtatgtactgttattgaaaatatatttaatacctttggaaaaatcttgggcaagatgacctactagttttccttgaaaaaaagttgctttgttattaatattgtgcttaaattatttttatacaccattgttccttacctttacataattgcaatatttcccccttactacttcttggaaaaaaattacaaaatgaagttttatagaaaagaaaaaaaa

SEQ ID NO: 39 - DNA, 3710 bp, Homo sapiens
aggacgcgtttccaagttccagtgactcctcctgtttgggactcggggggagagtgcggggagacaaataaaacctcgggcggcggcggctggtgggaagacttgaacttgaatctcgaaccactgcatctccgactctgcccagactcttcactccgcggcaccctcaaaccccagcccaggccggggcgcacaagccagccagcgcacctgcagtcctcgcccggacgcgccgcgccccctcggaaccaggctctgctccgagcagccttcgcccctcaagccagccacagtccccgccaggccgggtgggcgtcaagatgaaggcggcccgcttcgtgctgcgcagcgctggctcgctcaacggcgccggcctggtgccccgagaggtggagcatttctcgcgctacagcccgtccccgctgtccatgaagcagctactggactttggttcagaaaatgcatgtgaaagaacttcttttgcatttttgcgacaagaattgcctgtgagactcgccaacattctgaaggaaattgatatcctcccgacccaattagtaaatacctcttcagtgcaattggttaaaagctggtatatacagagcctgatggatttggtggaattccatgagaaaagcccagatgaccagaaagcattatcagactttgtagatacactcatcaaagttcgaaatagacaccataatgtagtccctacaatggcacaaggaatcatagagtataaagatgcctgtacagttgacccagtcaccaatcaaaatcttcaatatttcttggatcgattttacatgaaccgtatttctactcggatgctgatgaaccagcacattcttatatttagtgactcacagacaggaaacccaagccacattggaagcattgatcctaactgtgatgtggtagcagtggtccaagatgcctttgagtgttcaaggatgctctgtgatcagtattatttatcatctccagaattaaagcttacacaagtgaatggaaaatttccagaccaaccaattcacatcgtgtatgttccttctcacctccatcatatgctctttgaactatttaagaatgcaatgcgggcaacagttgaacaccaggaaaatcagccttcccttacaccaatagaggttattgttgtcttgggaaaagaagaccttaccattaagatttcagacagaggaggtggtgttcccctgagaattattgaccgcctctttagttatacatactccactgcaccaacgcctgtgatggataattcccggaatgctcctttggctggttttggttacggcttgccaatttctcgtctgtatgcaaagtactttcaaggagatctgaatctctactctttatcaggatatggaacagatgctatcatctacttaaaggctttgtcttctgagtctatagaaaaacttccagtttttaacaagtcagccttcaaacattatcagatgagctctgaggctgatgactggtgtatcccaagcagggaaccaaagaacctggcaaaagaagtggccatgtgaagagggacactcaggacactttacgggatcaaagtgggtctacaccagtgctgcttcctgaatgtttgtgtgtgaacccttgtttcctccaaaacaaacgacagcaacgaaaactccttaatcagaacactgatccaatgaggaatggagcttgtttctgtgacccaggagaacttagtgcaagactacaggagttaacagatggccagctccttattttttaatgtagaataactcctgagtttatatcaaatcctgaagaaataagcctcagttttccatctgtttttgataagaataagaaagggagtgagtgtgaagatggtggttagcagtttcactaagactgatattttaggcctcttgttcacatcaaaagatattggtgtcagaataccagcattttcctgccatgcaaaggattaaaacttagtttacactatgtggttacaaatatatgtcaatgtacattttgaacatatttatgtgctatggaaggaaatgctggtgactaaaataaggtttactctgaaagaggaggaattttattcaaagcattcaaacattttattcaagtgtttcaaaattcaaagcattgtattcaaagttgcagtgaaggcatcaacttatgtaaaaactcagaaggaaggctcctctgataaaaacacagctcctttattatgctgcttttcttgttcactttacacactaagtaaacacttattgtcaggtgcctagtcttgagtgaattgttagatgtgcactgaactcgggatgttggggattggagagagagaattgccaaagtaacagcaaaaatatctcttactttgctttgtttataaataaattagtagattggaaaaactagtgttagggaaagaaatcacatgttcagagcctaattcagtaggaagggcttttctctaccctgaaatgaaggtaatccaaaggcatccattttctaggcttaaaagatatatttttgatatatttaattatattctctacactccagcattaatatgtctgtttaaaaattactaattctcaaatggctcaagaacattagaatttaagtaccttttagagtaattattttaagcaaatagcctggacgtaagagattctcatgccagcatgctttcatttgtcagttgttgtgactgagagataatgaatgacacctgaaatgcatatggtatttttgggagagttaaggtataatttgaaggttggcagaccagttgcgctgattactcttagagaagaagaaatggaaaaatgaaagaaggcaggaaggaaagaaaggatataggaagagagggaagcagaaggcaggcatttttctattttccccacaaattatttcaaaaaaaatctgtattttctgggatatgtcattggcaagaggaagaactggtgttttgaaagcagtatggattctttaaatgcctctcactcttacaagatagtaggctttgagataataaacttacccgtgtcaattaacatttaaactggcatatagaaaaaaaggaggatttttctgcattgtaaaataatcagtatggtttatatgttgaatttgacatttgtgtgtaatttcatggtggcctagtgttgtggtgcttctggtaatggtaatagaagctcaactatttttttgtggatttcagtttttatcatcagaagtcctagacagtgacatttcttaatggtgggagtccagctcatgcatttctgattatacaaaacagtttgcagtaggttatttgtcatttcagttttttactgaaatttgagctaaacatttttacatgtaaatacttgtatttaccaaagatttaaatcagttgattaattaattaactcaaatactgtgaactatctctaaaacactagaaaaaagaaatgttagtatctcaattacaccaactgtgcaaatgaactttgataaaatagaaataatctacattggcctttgtgaaatctggggaagagctttaggattctagtagatggatactgaatactcaggcccacttaaattattaatgtatacattgtgtttttgtctttatgctatgtacagagaaatgtgataattttttataataaatattttttatgatgataaaagaaaaaaaaaaa

SEQ ID NO: 40 - DNA, 1295 bp, Homo sapiens
ccttcccctggcccggggagctgctccttgtgctgccgggaaggtcaaagtcccgcgcccaccaggagagctcggcaagtatataaggacagaggagcgcgggaccaagcggcggcgaaggaggggaagaagagccgcgaccgagagaggccgccgagcgtccccgccctcagagagcagcctcccgagacaggcacttgctggattctccaaaagtatctgcagtggctgttccaccaggagagcctcagcctgcctggaagatgccgagatcgtgctgcagccgctcgggggccctgttgctggccttgctgcttcaggcctccatggaagtgcgtggctggtgcctggagagcagccagtgtcaggacctcaccacggaaagcaacctgctggagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtgagggcacagcggggccccagggctaccctcccccaggaggtcgaccccaaagccccttgctctcccctgccctgctgccgcctcccagcctggggggtcgtggcagataatcagcctcttaaagctgcctgtagttaggaaataaaacctttcaaatttcacatccacctctgactttgaatgtaaactgtgtgaataaagtaaaaatacgtagccgtcaaataacagc

SEQ ID NO: 41 - DNA, 6318 bp, Homo sapiens
tagtaagacaggtgccttcagttcactctcagtaaggggctggttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacagaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacgcaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaacatgttccctagctgaggatgacagagggatggcgaatacctcatgggacagcgcgtccttccctaaagactattgcaagtcatacttaggaatttctcctactttacactctctgtacaaaaacaaaacaaaacaacaacaatacaacaagaacaacaacaacaataacaacaatggtttacatgaacacagctgctgaagaggcaagagacagaatgatatccagtaagcacatgtttattcatgggtgtcagctttgcttttcctggagtctcttggtgatggagtgtgcgtgtgtgcatgtatgtgtgtgtgtatgtatgtgtgtggtgtgtgtgcttggtttaggggaagtatgtgtgggtacatgtgaggactgggggcacctgaccagaatgcgcaagggcaaaccatttcaaatggcagcagttccatgaagacacgcttaaaacctagaacttcaaaatgttcgtattctattcaaaaggaaatatatatatatatatatatatatatatatatatatataaattaaaaaggaaagaaaactaacaaccaaccaaccaaccaaccaaccacaaaccaccctaaaatgacagccgctgatgtctgggcatcagcctttgtactctgtttttttaagaaagtgcagaatcaacttgaagcaagctttctctcataacgtaatgattatatgacaatcctgaagaaaccacaggttccatagaactaatatcctgtctctctctctctctctctctctctcttttttttttctttttccttttgccatggaatctgggtgggagaggatactgcgggcaccagaatgctaaagtttcctaacattttgaagtttctgtagttcatccttaatcctgacacccatgtaaatgtccaaaatgttgatcttccactgcaaatttcaaaagccttgtcaatggtcaagcgtgcagcttgttcagcggttctttctgaggagcggacaccgggttacattactaatgagagttgggtagaactctctgagatgtgttcagatagtgtaattgctacattctctgatgtagttaagtatttacagatgttaaatggagtatttttattttatgtatatactatacaacaatgttcttttttgttacagctatgcactgtaaatgcagccttcttttcaaaactgctaaatttttcttaatcaagaatattcaaatgtaattatgaggtgaaacaattattgtacactaacatatttagaagctgaacttactgcttatatatatttgattgtaaaaacaaaaagacagtgtgtgtgtctgttgagtgcaacaagagcaaaatgatgctttccgcacatccatcccttaggtgagcttcaatctaagcatcttgtcaagaaatatcctagtcccctaaaggtattaaccacttctgcgatatttttccacattttcttgtcgcttgtttttctttgaagttttatacactggatttgttaggggaatgaaattttctcatctaaaatttttctagaagatatcatgattttatgtaaagtctctcaatgggtaaccattaagaaatgtttttattttctctatcaacagtagttttgaaactagaagtcaaaaatctttttaaaatgctgttttgttttaatttttgtgattttaatttgatacaaaatgctgaggtaataattatagtatgatttttacaataattaatgtgtgtctgaagactatctttgaagccagtatttctttcccttggcagagtatgacgatggtatttatctgtattttttacagttatgcatcctgtataaatactgatatttcattcctttgtttactaaagagacatatttatcagttgcagatagcctatttattataaattatgagatgatgaaaataataaagccagtggaaattttctacctaggatgcatgacaattgtcaggttggagtgtaagtgcttcatttgggaaattcagcttttgcagaagcagtgtttctacttgcactagcatggcctctgacgtgaccatggtgttgttcttgatgacattgcttctgctaaatttaataaaaacttcagaaaaacctccattttgatcatcaggatttcatctgagtgtggagtccctggaatggaattcagtaacatttggagtgtgtattcaagtttctaaattgagattcgattactgtttggctgacatgacttttctggaagacatgatacacctactactcaattgttcttttcctttctctcgcccaacacgatcttgtaagatggatttcacccccaggccaatgcagctaattttgatagctgcattcatttatcaccagcatattgtgttctgagtgaatccactgtttgtcctgtcggatgcttgcttgattttttggcttcttatttctaagtagatagaaagcaataaaaatactatgaaatgaaagaacttgttcacaggttctgcgttacaacagtaacacatctttaatccgcctaattcttgttgttctgtaggttaaatgcaggtattttaactgtgtgaacgccaaactaaagtttacagtctttctttctgaattttgagtatcttctgttgtagaataataataaaaagactattaagagcaataaattatttttaagaaatcgagatttagtaaatcctattatgtgttcaaggaccacatgtgttctctattttgcctttaaatttttgtgaaccaattttaaatacattctcctttttgccctggattgttgacatgagtggaatacttggtttcttttcttacttatcaaaagacagcactacagatatcatattgaggattaatttatcccccctacccccagcctgacaaatattgttaccatgaagatagttttcctcaatggacttcaaattgcatctagaattagtggagcttttgtatcttctgcagacactgtgggtagcccatcaaaatgtaagctgtgctcctctcatttttatttttatttttttgggagagaatatttcaaatgaacacgtgcaccccatcatcactggaggcaaatttcagcatagatctgtaggatttttagaagaccgtgggccattgccttcatgccgtggtaagtaccacatctacaattttggtaaccgaactggtgctttagtaatgtggatttttttcttttttaaaagagatgtagcagaataattcttccagtgcaacaaaatcaattttttgctaaacgactccgagaacaacagttgggctgtcaacattcaaagcagcagagagggaactttgcactattggggtatgatgtttgggtcagttgataaaaggaaaccttttcatgcctttagatgtgagcttccagtaggtaatgattatgtgtcctttcttgatggctgtaatgagaacttcaatcactgtagtctaagacctgatctatagatgacctagaatagccatgtactataatgtgatgattctaaatttgtacctatgtgacagacattttcaataatgtgaactgctgatttgatggagctactttaagatttgtaggtgaaagtgtaatactgttggttgaactatgctgaagagggaaagtgagcgattagttgagcccttgccgggccttttttccacctgccaattctacatgtattgttgtggttttattcattgtatgaaaattcctgtgattttttttaaatgtgcagtacacatcagcctcactgagctaataaagggaaacgaatgtttcaaatcta

SEQ ID NO: 42 - DNA, 4790 bp, Homo sapiens
ggggaagcgcagtgcgcaggcgcaactgcctggctctgctcgctccggcgctccggcccagctctcgcggacaagtccagacatcgcgcgcccccccttctccgggtccgccccctcccccttctcggcgtcgtcgaagataaacaatagttggccggcgagcgcctagtgtgtctcccgccgccggattcggcgggctgcgtgggaccggcgggatcccggccagccggccatggcggggctgtactcgctgggagtgagcgtcttctccgaccagggcgggaggaagtacatggaggacgttactcaaatcgttgtggagcccgaaccgacggctgaagaaaagccctcgccgcggcggtcgctgtctcagccgttgcctccgcggccgtcgccggccgcccttcccggcggcgaagtctcggggaaaggcccagcggtggcagcccgagaggctcgcgaccctctcccggacgccggggcctcgccggcacctagccgctgctgccgccgccgttcctccgtggcctttttcgccgtgtgcgacgggcacggcgggcgggaggcggcacagtttgcccgggagcacttgtggggtttcatcaagaagcagaagggtttcacctcgtccgagccggctaaggtttgcgctgccatccgcaaaggctttctcgcttgtcaccttgccatgtggaagaaactggcggaatggccaaagactatgacgggtcttcctagcacatcagggacaactgccagtgtggtcatcattcggggcatgaagatgtatgtagctcacgtaggtgactcaggggtggttcttggaattcaggatgacccgaaggatgactttgtcagagctgtggaggtgacacaggaccataagccagaacttcccaaggaaagagaacgaatcgaaggacttggtgggagtgtaatgaacaagtctggggtgaatcgtgtagtttggaaacgacctcgactcactcacaatggacctgttagaaggagcacagttattgaccagattccttttctggcagtagcaagagcacttggtgatttgtggagctatgatttcttcagtggtgaatttgtggtgtcacctgaaccagacacaagtgtccacactcttgaccctcagaagcacaagtatattatattggggagtgatggactttggaatatgattccaccacaagatgccatctcaatgtgccaggaccaagaggagaaaaaatacctgatgggtgagcatggacaatcttgtgccaaaatgcttgtgaatcgagcattgggccgctggaggcagcgtatgctccgagcagataacactagtgccatagtaatctgcatctctccagaagtggacaatcagggaaactttaccaatgaagatgagttatacctgaacctgactgacagcccttcctataatagtcaagaaacctgtgtgatgactccttccccatgttctacaccaccagtcaagtcactggaggaggatccatggccaagggtgaattctaaggaccatatacctgccctggttcgtagcaatgccttctcagagaattttttagaggtttcagctgagatagctcgagagaatgtccaaggtgtagtcataccctcaaaagatccagaaccacttgaagaaaattgcgctaaagccctgactttaaggatacatgattctttgaataatagccttccaattggccttgtgcctactaattcaacaaacactgtcatggaccaaaaaaatttgaagatgtcaactcctggccaaatgaaagcccaagaaattgaaagaacccctccaacaaactttaaaaggacattagaagagtccaattctggccccctgatgaagaagcatagacgaaatggcttaagtcgaagtagtggtgctcagcctgcaagtctccccacaacctcacagcgaaagaactctgttaaactcaccatgcgacgcagacttaggggccagaagaaaattggaaatcctttacttcatcaacacaggaaaactgtttgtgtttgctgaaatgcatctgggaaatgaggtttttccaaacttaggatataagagggctttttaaatttggtgccgatgttgaactttttttaaggggagaaaattaaaagaaatatacagtttgactttttggaattcagcagttttatcctggccttgtacttgcttgtattgtaaatgtggattttgtagatgttagggtataagttgctgtaaaatttgtgtaaatttgtatccacacaaattcagtctctgaatacacagtattcagagtctctgatacacagtaattgtgacaatagggctaaatgtttaaagaaatcaaaagaatctattagattttagaaaaacatttaaactttttaaaatacttattaaaaaatttgtataagccacttgtcttgaaaactgtgcaactttttaaagtaaattattaagcagactggaaaagtgatgtattttcatagtgacctgtgtttcacttaatgtttcttagagccaagtgtcttttaaacattattttttatttctgatttcataattcagaactaaatttttcatagaagtgttgagccatgctacagttagtcttgtcccaattaaaatactatgcagtatctcttacatcagtagcatttttctaaaaccttagtcatcagatatgcttactaaatcttcagcatagaaggaagtgtgtttgcctaaaacaatctaaaacaattcccttctttttcatcccagaccaatggcattattaggtcttaaagtagttactcccttctcgtgtttgcttaaaatatgtgaagttttccttgctatttcaataacagatggtgctgctaattcccaacatttcttaaattattttatatcatacagttttcattgattatatgggtatatattcatctaataaatcagtgaactgttcctcatgttgctgaatttgtagttgttggtttattttaatggtatgtacaagttgagtatcccttatccaaaatgcttgggaccagaagtgtttcagattttttaaaattttggaatatttgctttatactgagcttttgagtgttcccaatctgaaattcaaaatgctctaatgagcatttcctttgagcatcatgcctgctctgaaaaagtttctgattctggagcattttggattttggattttcagattagggatgcttaacctggattaacattctgttgtgccatgatcatgctttacagtgagtgtattttatttatttattattttgtttgtttgtttgagatggagtctcactctgtcatccaggctagagtgcagtggcgtgatctcggctgactgcaacctctgcctcccgggttcaagtgattctcctgcctcaatctctctccccagaagctgggattacaggtgtgtgccaccacacccggctaatttttttttttttttttgagatggagtctagctctgtcatccaggctggagtgcagtggtgtgatctcggctccctgcaacctctgccttctgggttcctgcgattctcctgcctcagcctcctgagtagctgagattacaggcacgcgccactgtgcccagccaatttttgtatttttagtagagatggggtttcacatgtcagtcatgctggtcttgatctcctgacctcgtgatccacccgcctcgacctcccaaagtactgggattacaggcgtgagccaccgcatccggcctgagttttatgctttcaatgtatttcttacatttcagttcaagtgattttcatgtctcagcctcctgagtagctggaactacaggtgcgtgccaccatgcctggctaagttttgtatttttagtagagatgggttttcatcatgttggccaagatggtcttgatctcttgacctcatgatccaccagcctaggcctcccaaagtgctgggattacaggtgtgagccaccgtgcccagccaactatgccattatttaaccatgtccacacattctggttattttcaatattttgcagaagataattcttgatcggtgtgtcttatgccacaaggattaaaatatgtattcattgctacaaaacaatatctcgaaatttagcagtttaaaacaacaaatattatctccagtttctgagcctcagaaatctgagagtggtttagctgggtgatagtctcgtggttttggtcaagctaccaaccagggctacaatctttcgaaggtgtcattggggctagaagatctgcttcccgcaagactcacagctgttggcaggagacctcagtttgttgccacatgttcccctccagagggcctctcacaacatggcagttatttgtccccagagcaagcaacaccggagggcaaggaagaagccatgatgttttttgtaacctagcctctgaaagtgtcataccaattctgtattttgttggtcacacagaccaagtcaactacaacgtgggagactcctacacaaggcatgaattctaggaggtgggcatttttaagtgtcatctggaaggaggctgtcacaacctggaagttaaaagcattgatattctgaaatacagcgtgtataacattgttttagtagggtgtgcaatagttatgttttggtaatagcattaatgaacaatgttattttcatcttccagacatctggaagattgctctagtggagtaaaacatcttaatgtattttgtccctaaataaactatctcactaacaaaaaaaaaaaaaaa

SEQ ID NO: 43 - DNA, 1637 bp, homo sapiens
agagggcccgctcaccaccccgtaggccccgcccctgcgtctctgcccgccccgtggcgcccgagtgcactgaagatggcggctgctgtaggacggttgctccgagcgtcggttgcccgacatgtgagtgccattccttggggcatttctgccactgcagccctcaggcctgctgcatgtggaagaacgagcttgacaaatttattgtgttctggttccagtcaagcaaaattattcagcaccagttcctcatgccatgcacctgctgtcacccagcatgcaccctattttaagggtacagccgttgtcaatggagagttcaaagacctaagccttgatgactttaaggggaaatatttggtgcttttcttctatcctttggatttcacctttgtgtgtcctacagaaattgttgcttttagtgacaaagctaacgaatttcacgacgtgaactgtgaagttgtcgcagtctcagtggattcccactttagccatcttgcctggataaatacaccaagaaagaatggtggtttgggccacatgaacatcgcactcttgtcagacttaactaagcagatttcccgagactacggtgtgctgttagaaggttctggtcttgcactaagaggtctcttcataattgaccccaatggagtcatcaagcatttgagcgtcaacgatctcccagtgggccgaagcgtggaagaaaccctccgcttggtgaaggcgttccagtatgtagaaacacatggagaagtctgcccagcgaactggacaccggattctcctacgatcaagccaagtccagctgcttccaaagagtactttcagaaggtaaatcagtagatcacccatgtgtatctgcaccttctcaactgagagaagaaccacagttgaaacctgcttttatcattttcaagatggttatttgtagaaggcaaggaaccaattatgcttgtattcataagtattactctaaatgttttgtttttgtaattctggctaagaccttttaaacatggttagttgctagtacaaggaatcctttattggtaacatcttggtggctggctagctagtttctacagaacataatttgcctctatagaaggctattcttagatcatgtctcaatggaaacactcttctttcttagccttacttgaatcttgcctataataaagtagagcaacacacattgaaagcttctgatcaacggtcctgaaattttcatcttgaatgtctttgtattaaactgaattttcttttaagctaacaaagatcataattttcaatgattagccgtgtaactcctgcaatgaatgtttatgtgattgaagcaaatgtgaatcgtattattttaaaaagtggcagagtgacttaactgatcatgcatgatccctcatccctgaaattgagtttatgtagtcattttacttattttattcattagctaactttgtctatgtatatttctagatattgattagtgtaatcgattataaaggatatttatcaaatccagggattgcattttgaaattataattattttctttgctgaagtattcattgtaaaacatacaaaataaacatattttaaaacatttgcattttaccaccaaaaaaaaaaaaaaa

SEQ ID NO: 44 - DNA, 6582 bp, Homo sapiens
agagggcaaggagagagcagagaacacactttgccttctctttggtattgagtaatatcaaccaaattgcagacatctcaacactttggccaggcagcctgctgagcaaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaa

SEQ ID NO: 45 - DNA, 2457 bp, Homo sapiens
attagatcagtgttcataagaacatctgtaggcacacatacacactctctttacagtcagccttctgcttgccacagtcatagtgggcagtcagtgaatcttccccaagtgctgacaattaatacctggtttagcggcaaagattcagagaggcgtgagcagcccctctggccttcagacaaaaatctacgtaccatcagaaactatgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccgagggaaaagtatgggtgttctctttggaggacgctcatacatgccttctacccacagaaccacagaaaaatggaatagtgtagctgactgcctgccctgtgttttcctggtggattttgaatttgggtgtgctacatcatacattcttccagaacttcaggatgggctatcttttcatgtctctattgccaaaaatgacaccatctatattttaggaggacattcacttgccaataatatccggcctgccaacctgtacagaataagggttgatcttcccctgggtagcccagctgtgaattgcacagtcttgccaggaggaatctctgtctccagtgcaatcctgactcaaactaacaatgatgaatttgttattgttggtggctatcagcttgaaaatcaaaaaagaatgatctgcaacatcatctctttagaggacaacaagatagaaattcgtgagatggagaccccagattggaccccagacattaagcacagcaagatatggtttggaagcaacatgggaaatggaactgtttttcttggcataccaggagacaataaacaagttgtttcagaaggattctatttctatatgttgaaatgtgctgaagatgatactaatgaagagcagacaacattcacaaacagtcaaacatcaacagaagatccaggggattccactccctttgaagactctgaagaattttgtttcagtgcagaagcaaatagttttgatggtgatgatgaatttgacacctataatgaagatgatgaagaagatgagtctgagacaggctactggattacatgctgccctacttgtgatgtggatatcaacacttgggtaccattctattcaactgagctcaacaaacccgccatgatctactgctctcatggggatgggcactgggtccatgctcagtgcatggatctggcagaacgcacactcatccatctgtcagcaggaagcaacaagtattactgcaatgagcatgtggagatagcaagagctctacacactccccaaagagtcctacccttaaaaaagcctccaatgaaatccctccgtaaaaaaggttctggaaaaatcttgactcctgccaagaaatcctttcttagaaggttgtttgattagttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaaaaaaaaaaaaaaaaaaaaaaaaaaaa

SEQ ID NO: 46 - DNA, 4903 bp, Homo sapiens
gtcgtttgcggcggcgcaggcgcggtgcgggcggcggacgggcgggcgcttcgccgtttgaatggctgcgggcccgggccctcacctcacctgaggtccggccgcccaggggtgcgctatgccgtcgggaggtgaccagtcgccaccgcccccgcctccccctccggcggcggcagcctcggatgaggaggaggaggacgacggcgaggcggaagacgccgcgccgcctgccgagtcgcccacccctcagatccagcagcggttcgacgagctgtgcagccgcctcaacatggacgaggcggcgcgggccgaggcctgggacagctaccgcagcatgagcgaaagctacacgctggagggaaatgatcttcattggttagcatgtgccttatatgtggcttgcagaaaatctgttccaactgtaagcaaagggacagtggaaggaaactatgtatctttaactagaatcctgaaatgttcagagcagagcttaatcgaattttttaataagatgaagaagtgggaagacatggcaaatctacccccacatttcagagaacgtactgagagattagaaagaaacttcactgtttctgctgtaatttttaagaaatatgaacccatttttcaggacatctttaaataccctcaagaggagcaacctcgtcagcagcgaggaaggaaacagcggcgacagccctgtactgtgtctgaaattttccatttttgttgggtgctttttatatatgcaaaaggtaatttccccatgattagtgatgatttggtcaattcttatcacctgctgctgtgtgctttggacttagtttatggaaatgcacttcagtgttctaatcgtaaagaacttgtgaaccctaattttaaaggcttatctgaagattttcatgctaaagattctaaaccttcctctgaccccccttgtatcattgagaaactgtgttccttacatgatggcctagttttggaagcaaaggggataaaggaacatttctggaaaccctatattaggaaactttatgaaaaaaagctccttaagggaaaagaagaaaatctcactgggtttctagaacctgggaactttggagagagttttaaagccatcaataaggcctatgaggagtatgttttatctgttgggaatttagatgagcggatatttcttggagaggatgctgaggaggaaattgggactctctcaaggtgtctgaacgctggttcaggaacagagactgctgaaagggtgcagatgaaaaacatcttacagcagcattttgacaagtccaaagcacttagaatctccacaccactaactggtgttaggtacattaaggagaatagcccttgtgtgactccagtttctacagctacgcatagcttgagtcgtcttcacaccatgctgacaggcctcaggaatgcaccaagtgagaaactggaacagattctcaggacatgttccagagatccaacccaggctattgctaacagactgaaagaaatgtttgaaatatattctcagcatttccagccagacgaggatttcagtaattgtgctaaagaaattgccagcaaacattttcgttttgcggagatgctttactataaagtattagaatctgttattgagcaggaacaaaaaagactaggagacatggatttatctggtattctggaacaagatgcgttccacagatctctcttggcctgctgccttgaggtcgtcactttttcttataagcctcctgggaattttccatttattactgaaatatttgatgtgcctctttatcatttttataaggtgatagaagtattcattagagcagaagatggcctttgtagagaggtggtaaaacaccttaatcagattgaagaacagatcttagatcatttggcatggaaaccagagtctccactctgggaaaaaattagagacaatgaaaacagagttcctacatgtgaagaggtcatgccacctcagaacctggaaagggcagatgaaatttgcattgctggctcccctttgactcccagaagggtgactgaagttcgtgctgatactggaggacttggaaggagcataacatctccaaccacattatacgataggtacagctccccaccagccagcactaccagaaggcggctatttgttgagaatgatagcccctctgatggagggacgcctgggcgcatgcccccacagcccctagtcaatgctgtccctgtgcagaatgtatctggggagactgtttctgtcacaccagttcctggacagactttggtcaccatggcaaccgccactgtcacagccaacaatgggcaaacggtaaccattcctgtgcaaggtattgccaatgaaaatggagggataacattcttccctgtccaagtcaatgttggggggcaggcacaagctgtgacaggctccatccagcccctcagtgctcaggccctggctggaagtctgagctctcaacaggtgacaggaacaactttgcaagtccctggtcaagtggccattcaacagatttccccaggtggccaacagcagaagcaaggccagtctgtaaccagcagtagtaatagacccaggaagaccagctctttatcgcttttctttagaaaggtataccatttagcagctgtccgccttcgggatctctgtgccaaactagatatttcagatgaattgaggaaaaaaatctggacctgctttgaattctccataattcagtgtcctgaacttatgatggacagacatctggaccagttattaatgtgtgccatttatgtgatggcaaaggtcacaaaagaagataagtccttccagaacattatgcgttgttataggactcagccgcaggcccggagccaggtgtatagaagtgttttgataaaagggaaaagaaaaagaagaaattctggcagcagtgatagcagaagccatcagaattctccaacagaactaaacaaagatagaaccagtagagactccagtccagttatgaggtcaagcagcaccttgccagttccacagcccagcagtgctcctcccacacctactcgcctcacaggtgccaacagtgacatggaagaagaggagaggggagacctcattcagttctacaacaacatctacatcaaacagattaagacatttgccatgaagtactcacaggcaaatatggatgctcctccactctctccctatccatttgtaagaacaggctcccctcgccgaatacagttgtctcaaaatcatcctgtctacatttccccacataaaaatgaaacaatgctttctcctcgagaaaagattttctattacttcagcaacagtccttcaaagagactgagagaaattaatagtatgatacgcacaggagaaactcctactaaaaagagaggaattcttttggaagatggaagtgaatcacctgcaaaaagaatttgcccagaaaatcattctgccttattacgccgtctccaagatgtagctaatgaccgtggttcccactgaggttagtctcttgtattaaactcttcacaaaatctgtttagcagcagcctttaatgcatctagattatggagcttttttccttaatccagctgatgagttacagcctgttagtaacatgaggggacattttggtgagaaatgggacttaactccttccagtgtccttagaacattttaattcatcccaactgtctttttttccctaccattcagtgattactgtcaaggctgcttagaatccaaacttggatttttgactctggcaaagcttttagaaatactgcaagaaaatgatgtgtacccaaacgtgagcataggaggcttctgttgacgtactccaacagaagaactgtgtttcaagttcaatcctacctgttttgtggtcagctgtagtcctcataaaaagcaaaacaaaaattaggtattttgtcctaaaacacctggtaggagtgtgtgattttttgcattcctgacaaaggagagcacacccaggtttggaggtcctaggtcattagccctcgtctcccgttccctttgtgcacatcttccctctccccattcggtgtggtgcagtgtgaaaagtccttgattgttcgggtgtgcaatgtctgagtgaacctgtataagtggaggcactttagggctgtaaaatgcatgattttgtaacccagattttgctgtatatttgtgatagcactttctacaatgtgaactttattaaatacaaaacttccaggctaaacatccaatattttctttaatgcttttatatttttttaaaatgttaaaacccctatagccaccttttgggaatgttttaaattctccagttttttgttatatagggatcaaccagctaagaaaagattttaatcaagttgaattgaggggattaatatgaaaacttatgacctcttcctttaggagggagttatctaaaagaaatgtctattaaggtgatatatttaaaaatatttttgggtgttcctggcagtttaaaaaaattggttggagaatttaggtttttattagtaccatagtaccatttatacaaattagaaaatgttatttaacagctgaattatctatacatatctttattaatcactattgttccagcagttttcaagtcaaattaataatcttattagggagaaaattcaattgtaaattgaatcagtataaacaaagttactaggtaacttcatattgctgagagaaatatggaacttacattgttcaattagaatagtgttctgcaaaaatatttataaaacttctcaagatactgctactgtaattttatatgaagataagtgtatttttcaataaagcatttataaattaaaaaaaaaaaaaaaaaa

SEQ ID NO: 47 - DNA, 5189 bp, Homo sapiens
ggactgcgaaaggagcagggttgcggagctagggctccagcctgcggccgcgcattcttgcgtctggccagccgcgagctctaagggtcggccccgcccggtccgcccccgcggctccctgccaggctctcgcgggcgcgctcggggtggggcctcgcggctggcggagatgcggccggggctgcgcggtggtgatgcgagcctgctgggcggcgcgccggggcagccggagccgcgcgccgcggcgctgtaatcggacaccaagagcgctcgcccccggcctccggccactttccattcactccgaggtgcttgattgagcgacgcggagaagagctccgggtgccgcggcactgcagcgctgagattcctttacaaagaaactcagaggaccgggaagaaagaatttcacctttgcgacgtgctagaaaataaggtcgtctgggaaaaggactggagacacaagcgcatccaaccccggtagcaaactgatgacttttccgtgctgatttctttcaacctcggtattttcccttggatattaacttgcatatctgaagaaatggcattccggacaatttgcgtgttggttggagtatttatttgttctatctgtgtgaaaggatcttcccagccccaagcaagagtttatttaacatttgatgaacttcgagaaaccaagacctctgaatacttcagcctttcccaccatcctttagactacaggattttattaatggatgaagatcaggaccggatatatgtgggaagcaaagatcacattctttccctgaatattaacaatataagtcaagaagctttgagtgttttctggccagcatctacaatcaaagttgaagaatgcaaaatggctggcaaagatcccacacacggctgtgggaactttgtccgtgtaattcagactttcaatcgcacacatttgtatgtctgtgggagtggcgctttcagtcctgtctgtacttacttgaacagagggaggagatcagaggaccaagttttcatgattgactccaagtgtgaatctggaaaaggacgctgctctttcaaccccaacgtgaacacggtgtctgttatgatcaatgaggagcttttctctggaatgtatatagatttcatggggacagatgctgctatttttcgaagtttaaccaagaggaatgcggtcagaactgatcaacataattccaaatggctaagtgaacctatgtttgtagatgcacatgtcatcccagatggtactgatccaaatgatgctaaggtgtacttcttcttcaaagaaaaactgactgacaataacaggagcacgaaacagattcattccatgattgctcgaatatgtcctaatgacactggtggactgcgtagccttgtcaacaagtggaccactttcttaaaggcgaggctggtgtgctcggtaacagatgaagacggcccagaaacacactttgatgaattagaggatgtgtttctgctggaaactgataacccgaggacaacactagtgtatggcatttttacaacatcaagctcagttttcaaaggatcagccgtgtgtgtgtatcatttatctgatatacagactgtgtttaatgggccttttgcccacaaagaagggcccaatcatcagctgatttcctatcagggcagaattccatatcctcgccctggaacttgtccaggaggagcatttacacccaatatgcgaaccaccaaggagttcccagatgatgttgtcacttttattcggaaccatcctctcatgtacaattccatctacccaatccacaaaaggcctttgattgttcgtattggcactgactacaagtatacaaagatagctgtggatcgagtgaacgctgctgatgggagataccatgtcctgtttctcggaacagatcggggtactgtgcaaaaagtggttgttcttcctactaacaactctgtcagtggcgagctcattctggaggagctggaagtctttaagaatcatgctcctataacaacaatgaaaatttcatctaaaaagcaacagttgtatgtgagttccaatgaaggggtttcccaggtatctctgcaccgctgccacatctatggtacagcctgtgctgactgctgcctggcgcgggacccttattgcgcctgggatggccattcctgttccagattctacccaactgggaaacggaggagccgaagacaagatgtgagacatggaaacccactgactcaatgcagaggatttaatctaaaagcatacagaaatgcagctgaaattgtccagtatggagtaaaaaataacaccacttttctggagtgtgcccccaagtctccgcaggcatctatcaagtggctgttacagaaagacaaagacaggaggaaagaggttaagctgaatgaacgaataatagccacttcacagggactcctgatccgctctgttcagggttctgaccaaggactttatcactgcattgctacagaaaatagtttcaagcagaccatagccaagatcaacttcaaagttttagattcagaaatggtggctgttgtgacggacaaatggtccccatggacctgggccagctctgtgagggctttacccttccacccgaaggacatcatgggggcattcagccactcagaaatgcagatgattaaccaatattgcaaagacactcggcagcaacatcagcagggagatgaatcacagaaaatgagaggggactatggcaagttaaaggccctcatcaatagtcggaaaagtagaaacaggaggaatcagttgccagagtcataatattttcttatgtgggtcttatgcttccattaacaaatgctctgtcttcaatgatcaaattttgagcaaagaaacttgtgctttaccaaggggaattactgaaaaaggtgattactcctgaagtgagttttacacgaactgaaatgagcatgcattttcttgtatgatagtgactagcactagacatgtcatggtcctcatggtgcatataaatatatttaacttaacccagattttatttatatctttattcaccttttcttcaaaatcgatatggtggctgcaaaactagaattgttgcatccctcaattgaatgagggccatatccctgtggtattcctttcctgctttggggctttagaattctaattgtcagtgattttgtatatgaaaacaagttccaaatccacagcttttacgtagtaaaagtcataaatgcatatgacagaatggctatcaaaagaaatagaaaaggaagacagcatttaaagttgtataaaaacatgagttattcataaagagaaaatgatgagtttttatggttccaatgaaatatgttggggtttttttaagattgtaaaaataatcagttactggtatctgtcactgacctttgtttccttattcaggaagataaaaatcagtaacctaccccatgaagatatttggtgggagttatatcagtgaagcagtttggtttatattcttatgttatcaccttccaaacaaaagcacttactttttttggaagttatttattttagactcaaagaatataatctggcactactcagttattactgtttgttctcttattccctagtctgtgtggcaaattaaacaatataagaaggaaaaatttgaagtattagacttctaaataaggtgtgaaatcatcaaaaagaaaaatcaaagtagaaactactaattttttaagaggaatttataacaaatatggctagttttcaacttcagtactcaaattcaatgattcttccttttattaaaaccagtctcagatatcatactgatttttaagtcaacactatatattttatgatcttttcagtgtgatggcaaggtgcttgttatgtctagaaagtaagaaaacaatatgaggagacattctgtctttcaaaaggtaatggtacatacgttcactggtctctaagtgtaaaagtagtaaattttgtgatgaataaaataattatctcctaattgtatgttagaataattttattagaataatttcatactgaaattattttctccaaataaaaattagatggaaaaatgtgaaaaaaattattcatgctctcatatatattttaaaaacactacttttgcttttttatttaccttttaagacattttcatgcttccaggtaaaaacagatattgtaccatgtacctaatccaaatatcatataaacattttatttatagttaataatctatgatgaaggtaattaaagtagattatggcctttttaagtattgcagtctaaaacttcaaaaactaaaatcattgtcaaaattaatatgattattaatcagaatatcagaatatgattcactatttaaactatgataaattatgataatatatgaggaggcctcgctatagcaaaaatagttaaaatgctgacataacaccaaacttcattttttaaaaaatctgttgttccaaatgtgtataattttaaagtaatttctaaagcagtttattataatggtttgcctgcttaaaaggtataattaaacttcttttctcttctacattgacacacagaaatgtgtcaatgtaaagccaaaaccatcttctgtgtttatggccaatctattctcaaagttaaaagtaaaattgtttcagagtcacagttccctttatttcacataagcccaaactgatagacagtaacggtgtttagttttatactatatttgtgctatttaattctttctattttcacaattattaaattgtgtacactttcattacttttaaaaatgtagaaattcttcatgaacataactctgctgaatgtaaaagaaaattttttttcaaaaatgctgttaatgtatactactggtggttgattggttttattttatgtagcttgacaattcagtgacttaatatctattccatttgtattgtacataaaattttctagaaatacacttttttccaaagtgtaagtttgtgaatagattttagcatgatgaaactgtcataatggtgaatgttcaatctgtgtaagaaaacaaactaaatgtagttgtcacactaaaatttaattggatattgatgaaatcattggcctggcaaaataaaacatgttgaattccccaaaaaaaaaaaaaaaa

SEQ ID NO: 48 - DNA, 2164 bp, Homo sapiens
ataaatatcagagtgtgctgctgtggctttgtggagctgccagagtaaagcaaagagaaaggaagcaggcccgttggaagtggttgtgacaaccccagcaatgtggagaagcctggggcttgccctggctctctgtctcctcccatcgggaggaacagagagccaggaccaaagctccttatgtaagcaacccccagcctggagcataagagatcaagatccaatgctaaactccaatggttcagtgactgtggttgctcttcttcaagccagctgatacctgtgcatactgcaggcatctaaattagaagacctgcgagtaaaactgaagaaagaaggatattctaatatttcttatattgttgttaatcatcaaggaatctcttctcgattaaaatacacacatcttaagaataaggtttcagagcatattcctgtttatcaacaagaagaaaaccaaacagatgtctggactcttttaaatggaagcaaagatgacttcctcatatatgatagatgtggccgtcttgtatatcatcttggtttgcctttttccttcctaactttcccatatgtagaagaagccattaagattgcttactgtgaaaagaaatgtggaaactgctctctcacgactctcaaagatgaagacttttgtaaacgtgtatctttggctactgtggataaaacagttgaaactccatcgcctcattaccatcatgagcatcatcacaatcatggacatcagcaccttggcagcagtgagctttcagagaatcagcaaccaggagcaccaaatgctcctactcatcctgctcctccaggccttcatcaccaccataagcacaagggtcagcataggcagggtcacccagagaaccgagatatgccagcaagtgaagatttacaagatttacaaaagaagctctgtcgaaagagatgtataaatcaattactctgtaaattgcccacagattcagagttggctcctaggagctgatgctgccattgtcgacatctgatatttgaaaaaacagggtctgcaatcacctgacagtgtaaagaaaacctcccatctttatgtagctgacagggacttcgggcagaggagaacataactgaatcttgtcagtgacgtttgcctccagctgcctgacaaataagtcagcagcttatacccacagaagccagtgccagttgacgctgaaagaatcaggcaaaaaagtgagaatgaccttcaaactaaatatttaaaataggacatactccccaatttagtctagacacaatttcatttccagcatttttataaactaccaaattagtgaaccaaaaatagaaattagatttgtgcaaacatggagaaatctactgaattggcttccagattttaaattttatgtcatagaaatattgactcaaaccatattttttatgatggagcaactgaaaggtgattgcagcttttggttaatatgtctttttttttctttttccagtgttctatttgctttaatgagaatagaaacgtaaactatgacctaggggtttctgttggataattagcagtttagaatggaggaagaacaacaaagacatgctttccatttttttctttacttatctctcaaaacaatattactttgtcttttcaatcttctacttttaactaataaaataagtggattttgtattttaagatccagaaatacttaacacgtgaatattttgctaaaaaagcatatataactattttaaatatccatttatcttttgtatatctaagactcatcctgatttttactatcacacatgaataaagcctttgtatctttctttctctaatgttgtatcatactcttctaaaacttgagtggctgtcttaaaagatataaggggaaagataatattgtctgtctctatattgcttagtaagtatttccatagtcaatgatggtttaataggtaaaccaaaccctataaacctgacctcctttatggttaatactattaagcaagaatgcagtacagaattggatacagtacggatttgtccaaataaattcaataaaaaccttaaagctgaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - DNA, 3212 bp, Homo sapiens
gaacccggtggctgcacagacaaaaaagccccgaatggctggagggcgttcagctgttaacagccttttggggcagagcacggatttgacagctccacaacgtgaggatatccactgaccccgcgagacggaggagaacgcttccccgaaattctctgcccaccaaagccagcgctgcaaggttgcaactttcaaactttgtttttccagaaagaagactgccctttcgtgtacaaggagagggtgagagggtgacctagcttgtagatcggctgaaggcaccagtggttccaaatgtcacccagatgtgtgttttcatgacgatttgatttctctgattttatttttacatttttcattttaaaaatacaaagcaatttttttggggcatgctgaaaggtaactgaagaccgcaaaggaaaaactattgtcatggctgaaggagagaatgaagtgagatgggatggactctgcagcagagattcaactactagggagacagcattggaaaacattaggcaaaccattttgaggaaaaccgagtatcttcgttcggtgaaagaaacacctcatcgtccatcagacgggctttcaaataccgagtcttcggatgggttgaataagctacttgctcatctgcttatgctttctaagaggtgtcccttcaaagatgtgagagagaaaagtgagtttattctgaagagcatccaggaacttggcattagaattcctcgaccactaggacagggaccaagcagattcatcccagaaaaggagatcctccaagtggggagtgaagacgcacagatgcatgctttatttgcagattcttttgctgctttgggccgtttggataacattacgttagtgatggttttccacccacaatatttagaaagtttcttaaaaactcagcactatctactgcaaatggatgggccgttacccctacattatcgtcactacattggaataatggctgcggcaagacatcagtgctcctacttagtgaacctgcatgtaaatgatttccttcatgttggtggggaccccaagtggctcaatggtttagagaatgctcctcaaaaactacagaatttaggagaacttaacaaagtgttagcccatagaccttggcttattaccaaagaacacattgagggacttttaaaagctgaagagcacagctggtcccttgcggaattggtacatgcagtagttttactcacacactatcattctcttgcctcattcacattcggctgtggaatcagtccagaaattcattgtgatggtggccacacattcagacctccttctgttagcaactactgcatctgtgacattacaaatggcaatcacagtgtggatgagatgccggtcaactcagcagaaaatgtttctgtaagtgattctttctttgaggttgaagccctcatggaaaagatgaggcagttacaggaatgtcgagatgaagaagaggcaagtcaggaagagatggcttcacgttttgaaatagaaaaaagagagagtatgtttgtcttctcttcagatgatgaagaagttacaccagcaagagctgtatctcgtcattttgaggatactagttatggctataaagatttctctagacatgggatgcatgttccaacatttcgtgtccaggactattgctgggaagatcatggttattctttggtaaatcgcctttatccagatgtgggacagttgattgatgaaaaatttcacattgcttacaatcttacttataatacaatggcaatgcacaaagatgttgatacctcaatgcttagacgggcaatttggaactatattcactgcatgtttggaataagatatgatgattatgactatggtgaaattaaccagctattggatcgtagctttaaagtttatatcaaaactgttgtttgcactcctgaaaaggttaccaaaagaatgtatgatagcttctggaggcagttcaagcactctgagaaggttcatgttaatctgcttcttatagaagctaggatgcaagcagaactcctttatgctctgagagccattacccgctatatgacctgatgcctttccttcattaaagatgattctggaatgatcagcagatatagtctacaagggggaaggtactaagccccaggaccaatggtagacaaaataattcagaaatccattgtgccatgattcctttagtttctgctatttttctgtggaaaaccactgctggcacaagcagtgactgtttggcagcttcaagtttagagctgtgaagacaggctgccattcacagtattttgctttttgacagtacaagatgctgtgtaactgttttaatacagcaaatagtaactctccaaatcctgttgcttttatgttaaataagataacaagaattggagcatgcaaagaatgggacttggataatgacttaagctttatatgtaaagaattttagaagatcttggtgctgctattcctgctggaggaatgaatagatggctgtttcagttaagctattagtaataaaagtgaacattgctactatctgagcctacatacataacttgtgtgatttcaaattaaacttgcattatgtgttaattttcttgcatctaaaaaagcatagaattcctactcacacagctcagcaacaaccattttgatggtaacagttaatttctttcattagttttttaaattcagggttctggatattaaattaaaatggcattcttaaagattttcttcaaaaagcaatcctaaatgaaagtgtgtaaattataagaagctggcgatcttttgatatgctgtttcacaggatcctgacactggagggcagctgtcttgtgcattacttgtgtttccagcaccaaagttgtgggacatgttgctgtagactgctgcgcagtcctgggtgcattcagtctctctgcctctgcctgcctcctggtccccactttaaaggctgtgcagctccttaaataataaagctggaaaatatttttagtcgggttatcaaatttgatttacaaaaacgctaactttgtttgaaatgcaaacaggtttgaaaatatgtattaagtactttgtattctggaagcgtgaattgcttttgaagtctgtcagtattactggtatttttaaataaagaagaatttttctccaattttaaaaaaaaaaaaaaaaaa

SEQ ID NO: 50 - DNA, 4086 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttgggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctg

SEQ ID NO: 51 - DNA, 11579 bp, Homo sapiens
cggaccgtgctttcgccgcctgggagccgtccggcgcagcagtttctaggtccccactgtccccgccgtcccgccccttcgcgtcccgggaaccggctggcttccgagccgcactcgccgatcctccaggcatgccccgctacgagctggctttaatcctgaaagccatgcagcggggttggtacagtaggcttcactagacttagctgcaactcagaatttctcctccagcacctgagtaaatgctgatggtcttgtggagagtggattaagagtacgagctaagttctcaatcccaattaagaagcggaaaatttaaactgtcttcttcaaagtttatcacaaccaccaccatcaagacagcaaaccaaaggacaaagactttgaccctgctgtgttgctctgtgtagtccagttcacgtatggtttacagacttggctggggttactaaaaataaataaaaagttggacacttctgtcattggagcgctattattcacaagttaccagaatgagagctgtactggacacagcagacattgccatagtggccctgtattttatcctggtcatgtgcattggtttttttgccatgtggaaatctaatagaagcaccgtgagtggatacttcctggcggggcgctctatgacctgggtaacaattggtgcctctctgtttgtgagcaatattgggagtgagcacttcattgggctggcaggatctggagctgcaagtggatttgcagtgggcgcatgggaattcaatgccttactgcttttacaacttctgggatgggttttcatcccaatttacatccggtcaggggtatataccatgcctgaatacttgtccaagcgatttggtggccataggattcaggtctattttgcagccttgtctctgattctctatattttcaccaagctctcggtggatctgtattcgggtgccctttttatccaggagtctttgggttggaatctttatgtgtctgtcatcctgctcattggcatgactgctttgctgactgtcaccggaggccttgttgcagtgatctacacagacactctgcaggctctgctcatgatcattggggcacttacacttatgattattagcataatggagattggcgggtttgaggaagttaagagaaggtacatgttggcctcacccgatgtcacttccatcttattgacatacaacctttccaacacaaattcttgtaatgtctcccctaagaaagaagccctgaaaatgctgcggaatccaacagatgaagatgttccttggcctggattcattcttgggcagaccccagcttcagtatggtactggtgtgctgaccaagtcatcgtgcagagggtccttgcagccaaaaacattgctcatgccaaaggctctactcttatggctggcttcttaaagctcctgccaatgtttatcatagttgtcccaggaatgatttccaggatactgtttactgatgatatagcttgcatcaacccagagcactgcatgctggtgtgtggaagcagagctggttgctccaatattgcttacccacgcctggtgatgaagctggttcctgtgggccttcggggtttaatgatggcagtgatgattgcagctctgatgagtgacttagactctatctttaacagtgccagtaccatattcaccctcgatgtgtacaaacttatccgcaagagcgcaagctcccgggagttaatgattgtggggaggatatttgtggcatttatggtggtgatcagcatagcatgggtgccaatcatcgtggagatgcaaggaggccagatgtacctttacattcaggaggtagcagattacctgacacccccagtggcagccttgttcctgctggcaattttctggaagcgctgcaatgaacaaggggctttctatggtggaatggctggctttgttcttggagcagtccgtttgatactggcctttgcctaccgtgccccagaatgtgaccaacctgataataggccgggcttcatcaaagacatccattatatgtatgtggccacaggattgttttgggtcacgggactcattactgtaattgtgagccttctcacaccacctcccacaaaggaacagattcgaaccaccaccttttggtctaagaagaacctggtggtgaaggagaactgctccccaaaagaggaaccataccaaatgcaagaaaagagcattctgagatgcagtgagaataatgagaccatcaaccacatcattcccaacgggaaatctgaagacagcattaagggccttcagcctgaagatgttaatctgttggtaacctgcagagaggagggcaacccagtggcatccttaggtcattcagaggcagaaacaccagttgacgcttactccaatgggcaagcagctctcatgggtgagaaagagagaaagaaagaaacggatgatggaggtcggtactggaagttcatagactggttttgtggctttaaaagtaagagcctcagcaagaggagtctcagagacctgatggaagaggaggctgtttgtttacagatgctagaagagactcggcaagttaaagtaatactaaatattggactttttgctgtgtgttcacttggaattttcatgtttgtttatttctccttatgaacttaaggatatggtgagacactaacttaagacaatactgactggtctttggggaaaaaagttatgtaactgtgcatctctcaggcattgtttacgctgtaggttttagccaaattttacttagcagaaaatcatctaattacaagactttattttcccagagatggattaaagtaaatcttcaacttaagtgaagccaaacctaacagactgaattgtgcaaatgtggttttaaattttgcataccaaagtaagaagagaccaattattctcacagagcacttagagcagaatatatgttaagttaccatgaattaaggtatactgtctgcactgccaagtcttggcagaccttaccctgaagtagaagatttgctcatttctaaagttttttttctgtctctgtaatccctcctaccattaagaaaaacttatttcttagacattgtacaatcagttatgtactgaaaatcgaatgtgcttgtgtgatacttgtttcaggacaagttcatttgccaggttcattttgttagcatgagcctacggattctgatttcccaaagaaagaatgttttcctgtaggtatttttgtaccaccagtatatggaatgttagggaaaaactttgttccagttcctttttttttttctttctactttcaagtttaagtgaaccatactgaaatgaccaacaagtctgcctgtaaagttacatgtcatgattgtgttgttaaatgattatgggggagaaaatgaagtaaatgttgctgatgatccccatatttattgatcatattaaggttgtttatatagtttggaaatgaccagccccctaagcagtgtttgattaacttatgctaatcagatgattactcatatattctgctaattttctagctttattcttgttatttggaaaaattattagccaaatgccttcctaggtggatccagttggaagatatgtccagaaacctgaagaaaaattgacgctgcctttgtgtgctggattgctctacttgattagatcatgatatatcaaggttgaatttttagagggaaaatttaattctgatatcttattgcatccttgataagtttttccctgattttttttttcctcaaaagactttccatctgtacacagcctctacatttttgttgtagtgacttagagcataaggatgtttcagtgcaaactggccgtcggtaacagaaaactcagtgcatactttgctgttgttaggttgtcaatatagtctttctgtaggatggatagcatgtttgagaggtgccaaacaagaacttttggggttagtagtgtgtcttgtggagggtattacaggactgtgtaattataggactctaacttgacatggcttggcacccacttgcagctagtgggtacagggtacaaaagatgttagagaaaagctctacagattacgtacttctgtgtcttcgtatgctcaacactgtcctttgtcctccatgaaagatgaaggaagcaaattatgtatgtactttctttgaccttctttaatctctgatactttttagattgcatgattttactaggcttgtatttagggaaattactttcataaatacttttgtagattttgaatcaaaactcagtctttttaatttttttgtagtctataaactagtttcattatgatggacttgattagtccaaagttaattttagaaattgtcaggtagcatagtgtcttcccatgatcaggaggctttctgaaggactgagtctgtaaatgaaaaaataatttatgtatgaatagcatgtatttctgaagagcttagagtgccttgtagaatttttttctcaattttattcttgaggtttataatttgggggccaaatagatagagctcatcattttcttgtttggaagttgaggctgcgacatgtccaaggttatgaagtctcttttgggaagaacagaaaccaggtctccaaatctggactcatggtttgttcagatgtgtctggacaaatggttgtcaatgttttgtcctgttttttcaaaggaactgttcttcctttgggacaaccttttggtgtttgggaaagtaataagatcttggatttttcaaattaacattaagttgtaagaactaaaattttctttgaaccacattactgtgtaattcactgataattgacatattggctgggcagcctatctcttccatatccagcgtaaatgaataggaggtgtttgtgattttttttttctccctttatttaacattgagtcctagtagtttggagaattagggtccctctaccttctttctgctcttgtcttagtaagatacataaggtacatcatcttgtgtctgtgtgtatatagcagtaggtcaagtttagagtactaaagtctgtaaataaggaatgactattagcatattcattagaattgtttattcttgccagtataaacatcattttatttagactaaagtccctgaagcttgtctttcttattgcttcccagtaatagataatgtgctcgagtaagtttgtgaattgctgattgcaacttaattcagggaccagtcttcaatctatatttcattagaatgattgttcctggaatgatcatacatggactgtcttaagctagcaaaatgttcatactttacactgactaaatgggtcctaaatgatgacattggtctttagacattaacatgtgtatatttttatattagctcaagctaaggttcagaattgaagcttgatattgactagaatagctaaaagtcaaaatgaggtgaggacactggtcttggaaggtagagaaaaataaatgtcttaccaggtgttaatggtatccccagttcttagacttttgtcttctcaggcaattttcatctcaagatctgatgagaagggcatattacattggtatgcaggatgattattgcatattttgtgggacctctaatttccctggtcatctttcagaatattctgttctgccacccccagagagtaaacacttgagccgatttcttcttccccagctattctttcctgggggtaattatgctttgtctttagattagagaagcatcaagcaatagcaatggtgctgtgtccttcggcctaaattcaatagatctcatctcctagggcttccttttcacttggctcaaaggatccattgtattttggcacaaagagcctggccagggtcatgtagccatagctcttagggatgatacctcaagaaattagctgggacccatcactctgtgaaacttcacattttaagaactgagttgagggggttgttatgcacttctgtaacttgaggctaagcaaggggttaactcttgtgagagccaatagagtgtgtctgtattcgcagtccatggctcattttctttatagtaggcatatggatcttcccctctgactttgaatatcatttggtgtggcctgtgggttattttcattctttaccaccaaataaagcggcttattagctactcagttacttgctactcaaaggttaggtcttccctgttcctgcttggcagtgttaaagcttacagggttaacttatgatgattctcctggctcattttcatcagaggcatgatgactggaaagggatcacatgggtcgttggtggtgacacctcactgtttcctaggtttggatagagagatgtatacaagacctttcctgttaaattacgtgactacagagacttgccaggacaaaattttcctaagaaatcagaaaaatgattaagtgagataagtacctgggtgacacagatattagcccgttggtaaaagacaacaaatattagcttaaaatctgcatatgtagaatcattttcattagatttagagcttgaagcaccttggctctcagctactttaaactcctccccatataaatcagggcaccaataaataagtttcagctttttaaaccctggtttgatgttaagcattataaagtacgaagtttgttaccacagtagagataatttagtagaaaaatgctttgaggcttcagtatttgtaagattttgcattagccagatgctaggttgttgaaggcatttcagtgttgataatagcctgagcagacttctttacaaatgggatctgtttctatatgtgtatatgcccacttaccattcagagagactggtctttctctttgtcttccttcacattgctgtgtcagttctacacctagtcttttcagcacttagcaaattcaaattttgatttttttgtcagcttagttcactttaaggcatattggcatggtgtgtgaaagtgatgttttgccccagtattgaggacttttagatccaaataatgactcattaaatataattatgttttaagtatactgaatttctgttagcttaaaatgttaattctcaggaatgattttctcacactttgtgttggctaataataaaagcactgttttattctcaaaactcctttttcaaaaattagggagagagcagtagtgatcatttatgtgagcccctttgaaatgatggtgtcagagtgcagagaaacaatggagttttgatgccaaaaaggtttttttgcagtaaaagtaaaaatttggaattagttggcatatagaggaacccttttgtactggaacgtatgaggctggattgtgaaaaggtaatctttcgattgctagacttggttaacttagggctgcaaatctttttcttctgtcaaggtcacttaatatggaatgtttttgtcagactgtcctttgttggaatactttagctgttcagctactttgactcctaggagagaatttagttaaggttcaaagtaattaactggctttgccagtggtgagtcccacaccattattcacttagtagtcatataaatgtttttatttaaacttctctctcttcaatgctgagaataaggctttaaattactgattcacctttaaaggaatgttgtgagaattgatgtaatttctgtttctgtttccatctaaacttctttataaaaagagggattagtttttttgttttggggtaagcacctaatttatccagtaaccaacaaccctaaccattggcatatatagtctttcactcagaaataaacaaaaactgtttggtatatctgtatcattgctaatcttgtgcactttactttttgggcagtaccatacatagtctgaggctattgacttaaaccaataactgtactttatgtaatgactcttaaatttggttacctgggttcacagcttgcttgaagagaaaggatgctagaataaagtaagcagctgaagagcgagcaaatcaagacaaaacacagtggtctcagatttttcgtagtgtgggaacagtggttttgctctataccactgaaaagcactataacataattgttgtccatgatactgaagcttttcccctcacttctaggttgtttacattcagagctctatcaataagaggaatacatattacagtgaattcgacaaccgcacaagttggcagtaggtatccccaacctaatttatcttggtaaattcaccctgtttcctagtgctgctggataaaagagtgtttactttttattgctcttagacagagtagtctagataagttttcaatttatcaacatagcctagacttctgtaagtggaatgttcattagtaactcatctttttgttgttataattggaaacagaaacgaggcttattgctattgcagaaatcccaaactggcaaaggccagtatatatggtattccataatataaccagcttttgaaatttatgtgtttggattagtgccttctggttaccagtattgactctgctagtttgcacctttccgttcttaacagaaaatttgtatttgttattcctcttaaattttgtcgtaactagtgaaggaagtaaaaaaaaaaaaaaaacatgcattacattgacatactttatgtgcagcctttatttaggttcagtgaaaccaggtagttctgtatttgtgttgtagcctaaatgttgtttcttttatatccattaaaaacttaaagttacttatgttctgtgatcttaattttgttgtgtttccattgtaggttgataggtatatcgagaacaggtacgtgacaacagtttatattccatgatagaaagctaaagtccatagaaagcacaaaatcgtgttcacacattagtgtacccacacatagaaagcacaagactaatagtattctctgtatcccacaagtgccagtcataaaggccaccaggtatttgtctcagagttgctatgagcactacagtattgataagcccaagacaatgcggtatctaaactggtcctaatggtaagggacccaaaggaataatctcaataagtttgtaccacattgatggagggagagaatataaatgtcaagaatgccaaaattatatttgggggttactagctaaaatggggtttgagggctttttactgcaacttgaaactggagaaatagggacagatgtctaggtttttggtgggtggaacaggtgacatatttctgttttaagctgtagtgtgattggggttttttgtaaaaaatcttaaatcttttaggaaatattacctcttaacagtgcccccccaaacatgcagaaagtcatactttaacagggcaaatactacttgtctttgattttttttgtgtacgtttgtatgtgagagatgaagttacctttatttttttcctatacttgactgtgcttcattttaataaaggataatttgatctgagtgttctgagcatcagactaattctgaagcatatttgctagaggagctactttgcttttcacaatggggtggagaggattctttcacttgtcccattaaccctcttctagtctagatgagatgaaatctgttaatgtgtgtgtagaagaaaacgtatgttcttctactcagcattgcccttttccacctcctcacttcacctccgagtagcttgtttatcaagaatgaatgaatgtctttgtcttaaattttgcccatgtgttaaaagatgtaattctcagaatgggagagaaatgactacctttgttcctactcttttatataattatccttttagggaaagacttggtcaactctaatatatctagaaggaagactatatctggtgtagactaatatgagatgttttagaagagttaacctgaacactttgagggagagattattcttgccagcaaaaagctagccaggaatgagcctaccacattatttgagaatatcaaacctcaggcctggggggatgaggggaagaagattaccagaagtgcaggaaagagaagtttgaggaacacccttggcttagcaacatgtgataatgcaaagctgttataacctgttaatcctacgtactatgtgttctgtacctttacatgtttttaaatttaagatagtttgtaagaactgtacaaaaaaatgcttctggagatttctttggcagaaatgcctttcatctataatttcatggagaactgctttaattagcctaggtgaaaagtagtcctagcagtgtaaatatgtataattagagttttctaatttcactgtgagatctctaacttttgagtggcaaacagatcaagtcttttgctcatagacttttctgtggggttattaaaatgcaaaagctttattttttttaataatgccatactccattagtgtcagatgatggtatggaatttgttcccttgctttcccccactgttactgcttcagtttatagattgccagcagagttcagaaatagagcagggatttacccgttctttgcttggacatcccattttcttttgtccagacccatgttggcaatcatgtatgaactgtgttatacttctcagtgctttcttttttctttttgataagatggatatcaaaaatagttgctgtgcaaaagttagtagtcttcttcaagaagaaaaccaattctttttctaataatatcctgtgaaattgcttcattcattcatttatttttaagccaaatgtcagcagagtgctgctgcttttatctagtaattttgatatgtaagtattaatgcatttttaaaagatgtctacattgaaacatgttcttcccagtgtcctgcttatgatgctttgttcagattttttgtaagagaccagttagtacactgggggtgtatattgtgtacatgtgtcattttagttaggcattgtaggccaaatgtgattataaatgaagttgatgaacattaattttgttattagtgagttttttgaattgtaaatggatttccagtttaccttctgttgtctacagcttttttaattttaaggtttgactaattgtatccatctcattgtacagtgttttagttgcaagcagaaagtagaatttggtataaagcaggttatttctatattgaaaggagtacagttgaaattgtagatttaagattgttaaaatcatgacaattctaacttgtctattctaacctattgtgtacaatctgattttttaaaattgtaaacatgtatgatcttggtttcatgtgtttttgaaagtgttattgtttaaaaaatgaaaaaagcatatctgctaaagagctgtcagttttcattactgactctgtaaaatacactgttctttgtgtactgtgtgttattttgccagctgctgcattagccttcaaaagtatttggaaacttaagatgaactacatttcttgcaaagtacattcctttctgtggtattttgtcctgtaactgaagtatagtaattattttatggaaatgttagcaattctgtaccaactttgaataaaatgaaaaatttataaaa

SEQ ID NO: 52 - DNA, 8789 bp, Homo sapiens
atgctcagtggcttctcgacaagttggcagcaacaacacggccctggtcgtcgtcgccgctgcggtaacggagcggtttgggtggcggagcctgcgttcgcgccttcccgctctcctcgggaggcccttcctgctctcccctaggctccgcggccgcccagggggtgggagcgggtgaggggagccaggcgcccagcgagagaggccccccgccgcagggcggcccgggagctcgaggcggtccggcccgcgcgggcagcggcgcggcgctgaggaggggcggcctggccgggacgcctcggggcgggggccgaggagctctccgggccgccggggaaagctacgggcccggtgcgtccgcggaccagcagcgcgggagagcggactcccctcgccaccgcccgagcccaggttatcctgaatacatgtctaacaattttccttgcaacgttagctgttgtttttcactgtttccaaaggatcaaaattgcttcagaaattggagacatatttgatttaaaaggaaaaacttgaacaaatggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactgaggtcttttaccgttggggcccttaaccttatcaggatggtggactacaaaatacaatcctgtttataatctgaagatatatttcacttttgttctgctttatcttttcataaagggttgaaaatgtgtttgctgccttgctcctagcagacagaaactggattaaaacaattttttttttcctcttcagaacttgtcaggcatggctcagagcttgaagattaggagaaacacattcttattaattcttcacctgttatgtatgaaggaatcattccagtgctagaaaatttagccctttaaaacgtcttagagccttttatctgcagaacatcgatatgtatatcattctacagaataatccagtattgctgattttaaaggcagagaagttctcaaagttaattcacctatgttattttgtgtacaagttgttattgttgaacatacttcaaaaataatgtgccatgtgggtgagttaattttaccaagagtaactttactctgtgtttaaaaagtaagttaataatgtattgtaatctttcatccaaaatattttttgcaagttatattagtgaagatggtttcaattcagattgtcttgcaacttcagttttatttttgccaaggcaaaaaactcttaatctgtgtgtatattgagaatcccttaaaattaccagacaaaaaaatttaaaattacgtttgttattcctagtggatgactgttgatgaagtatacttttcccctgttaaacagtagttgtattcttctgtatttctaggcacaaggttggttgctaagaagcctataagaggaatttcttttccttcattcatagggaaaggttttgtattttttaaaacactaaaagcagcgtcactctacctaatgtctcactgttctgcaaaggtggcaatgcttaaactaaataatgaataaactgaatattttggaaactgctaaattctatgttaaatactgtgcagaataatggaaacattacagttcataataggtagtttggatatttttgtacttgatttgatgtgactttttttggtataatgtttaaatcatgtatgttatgatattgtttaaaattcagtttttgtatcttggggcaagactgcaaacttttttatatcttttggttattctaagccctttgccatcaatgatcatatcaattggcagtgactttgtatagagaatttaagtagaaaagttgcagatgtattgactgtaccacagacacaatatgtatgctttttacctagctggtagcataaataaaactgaatctcaacatacaaagttgaattctaggtttgatttttaagattttttttttcttttgcacttttgagtccaatctcagtgatgaggtaccttctactaaatgacaggcaacagccagttctattgggcagctttgtttttttccctcacactctaccgggacttccccatggacattgtgtatcatgtgtagagttggttttttttttttttaatttttattttactatagcagaaatagacctgattatctacaagatgataaatagattgtctacaggataaatagtatgaaataaaatcaaggattatctttcagatgtgtttacttttgcctggagaacttttagctatagaaacacttgtgtgatgatagtcctccttatatcacctggaatgaacacagcttctactgccttgctcagaaggtcttttaaatagaccatcctagaaaccactgagtttgcttatttctgtgatttaaacatagatcttgatccaagctacatgacttttgtctttaaataacttatctaccacctcatttgtactcttgattacttacaaattctttcagtaaacacctaattttcttctgtaaaagtttggtgatttaagttttattggcagttttataaaaagacatcttctctagaaattgctaactttaggtccattttactgtgaatgaggaataggagtgagttttagaataacagatttttaaaaatccagatgatttgattaaaaccttaatcatacattgacataattcattgcttcttttttttgagatatggagtcttgctgtgttgcccaggcaggagtgcagtggtatgatctcagctcactgcaacctctgcctcccgggttcaactgattctcctgcctcagcctccctggtagctaggattacaggtgcccgccaccatgcctggctaacttttgtagttttagtagagacggggttttgcctgttggccaggctggtcttgaactcctgacctcaagtgatccatccaccttggcctcccaaagtgctgggattacgggcgtgagccactgtccctggcctcattgttcccttttctactttaaggaaagttttcatgtttaatcatctggggaaagtatgtgaaaaatatttgttaagaagtatctctttggagccaagccacctgtcttggtttctttctactaagagccataaagtatagaaatacttctagttgttaagtgcttatatttgtacctagatttagtcacacgcttttgagaaaacatctagtatgttatgatcagctattcctgagagcttggttgttaatctatatttctatttcttagtggtagtcatctttgatgaataagactaaagattctcacaggtttaaaattttatgtctactttaagggtaaaattatgaggttatggttctgggtgggttttctctagctaattcatatctcaaagagtctcaaaatgttgaatttcagtgcaagctgaatgagagatgagccatgtacacccaccgtaagacctcattccatgtttgtccagtgcctttcagtgcattatcaaagggaatccttcatggtgttgcctttattttccggggagtagatcgtgggatatagtctatctcatttttaatagtttaccgcccctggtatacaaagataatgacaataaatcactgccatataaccttgctttttccagaaacatggctgttttgtattgctgtaaccactaaataggttgcctataccattcctcctgtgaacagtgcagatttacaggttgcatggtctggcttaaggagagccatacttgagacatgtgagtaaactgaactcatattagctgtgctgcatttcagacttaaaatccatttttgtggggcagggtgtggtgtgtaaaggggggtgtttgtaatacaagttgaaggcaaaataaaatgtcctgtctcccagatgatatacatcttattatttttaaagtttattgctaattgtaggaaggtgagttgcaggtatctttgactatggtcatctggggaaggaaaattttacattttactattaatgctccttaagtgtctatggaggttaaagaataaaatggtaaatgtttctgtgcctggtttgatggtaactggttaatagttactcaccattttatgcagagtcacattagttcacaccctttctgagagccttttgggagaagcagttttattctctgagtggaacagagttctttttgttgataatttctagtttgctcccttcgttattgccaactttactggcattttatttaatgatagcagattgggaaaatggcaaatttaggttacggaggtaaatgagtatatgaaagcaattacctctaaagccagttaacaattattttgtaggtggggtacactcagcttaaagtaatgcatttttttttcccgtaaaggcagaatccatcttgttgcagatagctatctaaataatctcatatcctcttttgcaaagactacagagaataggctatgacaatcttgttcaagcctttccatttttttccctgataactaagtaatttctttgaacataccaagaagtatgtaaaaagtccatggccttattcatccacaaagtggcatcctaggcccagccttatccctagcagttgtcccagtgctgctaggttgcttatcttgtttatctggaatcactgtggagtgaaattttccacatcatccagaattgccttatttaagaagtaaaacgttttaatttttagcctttttttggtggagttatttaatatgtatatcagaggatatactagatggtaacatttctttctgtgcttggctatctttgtggacttcaggggcttctaaaacagacaggactgtgttgcctttactaaatggtctgagacagctatggttttgaatttttagttttttttttttaacccacttcccctcctggtctcttccctctctgataattaccattcatatgtgagtgttagtgtgcctccttttagcattttcttcttctctttctgattcttcatttctgactgcctaggcaaggaaaccagataaccaaacttactagaacgttctttaaaacacaagtacaaactctgggacaggacccaagacactttcctgtgaagtgctgaaaaagacctcattgtattggcatttgatatcagtttgatgtagcttagagtgcttcctgattcttgctgagtttcaggtagttgagatagagagaagtgagtcatattcatattttcccccttagaataatattttgaaaggtttcattgcttccacttgaatgctgctcttacaaaaactggggttacaagggttactaaattagcatcagtagccagaggcaataccgttgtctggaggacaccagcaaacaacacacaacaaagcaaaacaaaccttgggaaactaaggccatttgttttgttttggtgtcccctttgaagccctgccttctggccttactcctgtacagatatttttgacctataggtgcctttatgagaattgagggtctgacatcctgccccaaggagtagctaaagtaattgctagtgttttcagggattttaacatcagactggaatgaatgaatgaaactttttgtcctttttttttctgtttttttttttctaatgtagtaaggactaaggaaaacctttggtgaagacaatcatttctctctgttgatgtggatacttttcacaccgtttatttaaatgctttctcaataggtccagagccagtgttcttgttcaacctgaaagtaatggctctgggttgggccagacagttgcactctctagtttgccctctgccacaaatttgatgtgtgacctttgggcaagtcatttatcttctctgggccttagttgcctcatctgtaaaatgagggagttggagtagattaattattccagctctgaaattctaagtgaccttggctaccttgcagcagttttggatttcttccttatctttgttctgctgtttgagggggctttttacttatttccatgttattcaaaggagactaggcttgatattttattactgttcttttatggacaaaaggttacatagtatgcccttaagacttaattttaaccaaaggcctagcaccaccttaggggctgcaataaacacttaacgcgcgtgcgcacgcgcgcgcgcacacacacacacacacacacacacacacacaggtcagagtttaaggctttcgagtcatgacattctagcttttgaattgcgtgcacacacacacgcacgcacacactctggtcagagtttattaaggctttcgagtcatgacattatagcttttgagttggtgtgtgtgacaccaccctcctaagtggtgtgtgcttgtaattttttttttcagtgaaaatggattgaaaacctgttgttaatgcttagtgatattatgctcaaaacaaggaaattcccttgaaccgtgtcaattaaactggtttatatgactcaagaaaacaataccagtagatgattattaactttattcttggctctttttaggtccattttgattaagtgacttttggctggatcattcagagctctcttctagcctacccttggatgagtacaattaatgaaattcatattttcaaggacctgggagccttccttggggctgggttgagggtggggggttggggagtcctggtagaggccagctttgtggtagctggagaggaagggatgaaaccagctgctgttgcaaaggctgcttgtcattgatagaaggactcacgggcttggattgattaagactaaacatggagttggcaaactttcttcaagtattgagttctgttcaatgcattggacatgtgatttaagggaaaagtgtgaatgcttatagatgatgaaaacctggtgggctgcagagcccagtttagaagaagtgagttgggggttggggacagatttggtggtggtatttcccaactgtttcctcccctaaattcagaggaatgcagctatgccagaagccagagaagagccactcgtagcttctgctttggggacaactggtcagttgaaagtcccaggagttcctttgtggctttctgtatacttttgcctggttaaagtctgtggctaaaaaatagtcgaacctttcttgagaactctgtaacaaagtatgtttttgattaaaagagaaagccaactaaaaaaaaaaaaaaaaaaaa

SEQ ID NO: 53 - DNA, 1593 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 54 - DNA, 3286 bp, Homo sapiens
gcgtgtcgggcgcggaagggggaggcggcccggggcgcccgcgagtgaggcgcggggcggcgaagggagcgcgggtggcggcacttgctgccgcggccttggatgggctgggcccccctcgccgctccgcctcctccacacgcgcggcggccgcggcgagggggacgcgccgcccggggcccggcaccttcgggaaccccccggcccggagcctgcggcctgcgccgcctcggccgccgggagccccgtggagcccccgccgccgcgccgccccgcggaccggacgctgagggcactcggggcggggcgcgcgctcgggcagacgtttgcggggaggggggcgcctgccgggccccggcgaccaccttgggggtcgcgggccggctcggggggcgcccagtgcgggccctcgcgggcgccgggcagcgaccagccctgagcggagctgttggccgcggcgggaggcctcccggacgcccccagccccccgaacgctcgcccgggccggcgggagtcggcgccccccgggaggtccgctcggtcgtccgcggcggagcgtttgctcctgggacaggcggtgggaccggggcgtcgccggagacgcccccagcgaagttgggctctccaggtgtgggggtcccggggggtagcgacgtcgcggacccggcctgtgggatgggcggcccggagaagactgcgctcggccgtgttcatacttgtccgtgggcctgaggtccccggaggatgacctagcactgaaaagccccggccggcctccccagggtccccgaggacgaagttgaccctgaccgggccgtctcccagttctgaggcccgggtcccactggaactcgcgtctgagccgccgtcccggacccccggtgcccgccggtccgcagaccctgcaccgggcttggactcgcagccgggactgacgtgtagaacaatcgtttctgttggaagaagggtttttcccttccttttggggtttttgttgccttttttttttcttttttctttgtaaaattttggagaagggaagtcggaacacaaggaaggaccgctcacccgcggactcagggctggcggcgggactccaggaccctgggtccagcatggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtgaggctggccgcctgcagcccgtgtccaggagccccgccaggtgcccgcgccaggccctcagtcttcctgccggttccgcccgccctcccggagaggtggccgccatgcttctgtgccgaccacgccccaggacctccggagcgccctgcagggccgggcagggggacagcagggaccgggcgcagccctcccccctcggccgcccggcagtgcacgcggcttgttgacttcgcagccccgggcggagccttcccgggcgggcgtgggaggagggaggcggcctccatgcactttatgtggagactactggccccgcccgtggcctcgtgctccgcagggcgcccagcgccgtccggcggccccgccgcagaccagctggcgggtgtggagaccaggctcctgaccccgccatgcatgcagcgccacctggaagccgcgcggccgctttggttttttgtttggttggttccattttctttttttctttttttttttaagaaaaaataaaaggtggatttgagctgtggctgtgaggggtgtttgggagctgctgggtggcaggggggctgtggggtcgggctcacgtcgcggccgcctttgcgctctcgggtcaccctgctttggcggcccggccggagggcaggaccctcacctctcccccaaggccactgcgctcttgggaccccagagaaaacccggagcaagcaggagtgtgcggtcaatatttatatcatccagaaaagaaaaacacgagaaacgccatcgcgggatggtgcagacgcggcggggactcggagggtgccgtgcgggcgaggccgcccaaatttggcaataaataaagcttgggaagcttggacctgaaaaaaaaaa

SEQ ID NO: 55 - DNA, 5174 bp, Homo sapiens
ggagagctcgccagagcgctcgcatggcgggccggtgattgtagtcaatctggccgtattctcaggcagggtcgcccggggcggactacatctcccgggatgctgcgcggccgccccgcggaagattgtgaatatgtatcagaatgttaatgattagctgctgctaaatttggtcaaagaagtcacctacacagagcgtgttgttagagctgtgctgagcgggtgtttgggttgttggctgctttcttccccctttctcacacacttgtatattattttgaggtggtgttcgcagagtttgaaaggagagagaattaaaaaaaaaagccgcaagcgtttcactcttttatttttataatccccttcaatttggggttaaaaaaaagacaagaaaacaggaaggaagagaaataaggaaatgagatgtggtaaaagaagctaaaaggtgccttttaaaagatcgttgctgtgaagtgaaaaaaatctccagagaaaccaaaaagcaccgccgagacctcttccgaaccaaaggagtttgtgtttgcttttagggaagaagaaagatcattcattcggaggaataacaaccaattaaaagacaaataaaaaaagtttggagtgggacgcagagcgagcgagaggagctgccggcgggcggtggggcgcggagcccgcactttcccggccgggtgagcggcggccgcggcgccgggctcggcgggtgcgcctcggcggagcgaacgtcggagcgttgccttgggagacgcgcgccggacaatgcccgcggcgggccagtgacgcccgcggggaatgcggagcggcccggcagccggcacccagccgccgccgcgcgttcctgccgcccgtgtcacgcgagacccggcgggggccgggaccgcccgagccgcccctcagaccgagccggccgcctccgctgccgcggccgcctcctcttcggggtcattaaagccaatgagccgcgcgcctctgccgagcgcagccaactaaatcggcttggatgattcgcgacctgagcaagatgtacccgcagaccagacacccggcaccgcatcagcctgctcaaccctttaaatttacaatttccgaatcctgtgatcggattaaggaagagtttcagtttttacaggctcaataccacagtctgaagctggaatgtgagaaactcgccagtgagaagacagagatgcagcggcattatgtcatgtattatgaaatgtcctatgggttgaatatagaaatgcacaagcaggcagagattgtcaagaggctgaatgctatctgtgcacaagtcattcctttcctgtcccaagagcaccagcaacaagtggtgcaggctgtggaacgggccaagcaggtgaccatggcagaactgaacgccatcattgggcaacaactccaggcccagcatttatcacatggacatggtctccccgtacctctgactccacacccttcagggctccagccccctgccattccacccatcggtagcagtgccgggcttctggccctctccagtgctctaggaggtcagtcccatcttccaattaaagatgagaagaagcaccatgacaatgatcaccaaagagacagagactccatcaagagctcttcagtatccccatcagccagtttccgaggtgctgagaagcacagaaactccgcagactactcctcagagagcaaaaagcagaaaactgaagaaaaggaaattgcagctcgttatgacagcgatggtgagaaaagtgatgacaacttggtggttgacgtttccaatgaggatccatcttcccctcgagggagcccagcacattcccccagagagaatggcctagacaagacacgcctgctcaagaaagatgccccgattagtccagcctctattgcatcttccagcagtactccctcctccaaatccaaagaacttagccttaagagggatatggggaaattgagtgaaacacgtcttagcgaagatgaacaatgcacattggggttacagagatggttttgtcgcctgtggtttatgaatgaaaaatctactactcccgtctcaaagtccaatacccctactccacgaactgatgcgcccaccccaggcagtaactctactcccggattgaggcctgtacctggaaaaccaccaggagttgaccctttggcctcaagcctaaggaccccaatggcagtaccttgtccatatccaactccatttgggattgtgccccatgctggaatgaacggagagctgaccagccccggagcggcctacgctgggctccacaacatctcccctcagatgagcgcagctgctgccgccgccgctgctgctgctgcctatgggagatcaccagtggtgggatttgatccacaccatcacatgcgtgtgccagcaatacctccaaacctgacaggcattccaggaggaaaaccagcatactccttccatgttagcgcagatggtcagatgcagcctgtcccttttccacccgacgccctcatcggacctggaatcccccggcatgctcgccagatcaacaccctcaaccacggggaggtggtgtgcgcggtgaccatcagcaaccccacgagacacgtgtacacgggtgggaagggctgcgtcaaggtctgggacatcagccacccaggcaataagagtcctgtctcccagctcgactgtctgaacagggataactacatccgttcctgcagattgctccctgatggtcgcaccctaattgttggaggggaagccagtactttgtccatttgggacctggcggctccaaccccacgcatcaaggcagagctgacatcctcggcccccgcctgctatgccctggccatcagccccgattccaaggtctgcttctcatgctgcagcgacggcaacatcgctgtgtgggatctgcacaaccagaccttggtgaggcaattccagggccacacagatggagccagctgtattgacatttctaatgatggcaccaagctctggacaggtggtttggacaacacggtcaggtcctgggacctgcgcgaggggcggcagctgcagcagcacgacttcacctcccagatcttttctctgggctactgcccaactggagagtggcttgcagtggggatggagaacagcaatgtggaagttttgcatgtcaccaagccagacaaataccaactacatcttcatgagagctgtgtgctgtcgctcaagtttgcccattgtggcaaatggtttgtaagcactggaaaggacaaccttctgaatgcctggagaacaccttatggggccagtatattccagtccaaagaatcctcatcggtgcttagctgtgacatctccgtggacgacaaatacattgtcactggctctggggataagaaggccacagtttatgaagttatttattaaagacaaatcttcatgcagactggacttctcctcctggtagcactttgctctgtcatcctttttgttcacccccatccccgcatctaaaaccaaggatttcagatactcattgcagttgtggagtttaatcccctttcttaacctcacttcccacttgctattgaattgtgaatagtcattaaaaacctgtgataccaaatcttcagctgtctacttggaagaacatggaataagcatacttaacagtgaaaagaatctttaattatgtattatatctgtaatatatttattttgtttaaagaaggctttctaacaatgactgactaaataaagctgtctgctcctgcattgataatgaaggtgcgttgtatttgatacccctccccccctttttttggcaaaggaggggaaaggaaggtttaaaataattgatttaaaatgtcactaagtgtagactgatgactgtatagagatgtgaaatgtataattacacatggaagcaatatgttgctgtgttgttattaggttttttttgtttttgttttctacatcttttaaagacttttggaaatttggctgaacaattagaacacaacaggccaactcatactcatttggatctatttagacaacgttaaccaatatatctatagctttagattatattcgataaaagtaattggactttttttctttttttgactcgttgacaagtgtctttgtaatatgtttttagttccctttttttgttgtattataggcagatgaacaaattaaatttggcctcaaagagagaacttactcccttctggatatttttgccacatttctttgcaaaaggagatatatatatctttagtcagttttgttgttatgagaaattatgggttattttgtggcatgctctttgggagctgcacagttatggggaggactcccactgctgtgcaagttaagtcttttacaaaacaaggacagcagaggagggtttgcagagacctccctctgaaaaacacaaagaatggactctctcctgggatgaggacttgctttctttacctccggttctttccatgtcttagttggatgtccctgaaatggacacaggctgtgccattgtgccagaaacattgtgttatcttttatgttgttgttgttgctgttaaactataatatgtgacttctttttttattattttttgtttgaatgctttaaaaatcttttaagtctgtggatctgctgatgtacagtgcctttgctgctatggatcaaaatcaaaagaaccgtgtagatatactttattgtataagtagaaaattacttaatttcatactagaaatggatggatgctgcaagttgaaatggactgtccattgacgttcctaatgtggtagcagaaaaaaaaaaatggtgtcttaagtgcttagtgtttgatgtcattaacagtttcgtaaaactctacagtgtagaaagattttgatactaaactgtgcgttgtacatagttctaatgcattgtattgaccaccagtacttctataatggtagattgtttgtgaattcagacttttaagcattaaacataaataacttctagtatgcttatttttctaattctttgtcttgatgacattagtttattttttatctttggctgtgccactcctatatattaaaaatgcctagttttttcaagggagattgttgttaaagtaaagtggttttttttgttgttaaa

SEQ ID NO: 56 - DNA, 1769 bp, Homo sapiens
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 57 - DNA, 2979 bp, Homo sapiens
gtggctcttctggcccgggctactatatagagacgtttccgcctcctgcttgaaactaacccctctttttctccaaaggagtgcttgtggagatcggatcttttctccagcaattgggggaaagaaggctttttctctgaattagcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttacgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgcccaaccctgtgatcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtattatgtatctttttgtatggtaaagactatattttgtacttaaccagatatatttttaccccagatggggatattctttgtaaaaaatgaaaataaagtttttttaatggaaaaaaaaaaaaaaaaaaaaaaaaaaaaaaa

(19) United States
(12) Patent Application Publication
(10) Pub. No.: US 2019/0376142 A1
(43) Pub. Date: Dec. 12, 2019

(54) METHOD TO DISTINGUISH TUMOR SUPRESSIVE FOXO ACTIVITY FROM OXIDATIVE STRESS

(71) Applicant: KONINKLIJKE PHILIPS N.V., EINDHOVEN (NL)

(72) Inventors: Anja VAN DE STOLPE, VUGHT (NL); Rick VELTER, EINDHOVEN (NL); Wilhelmus Franciscus Johannes VERHAEGH, Heusden gem. Asten (NL); Hendrik Jan VAN OOIJEN, Wijk en Aalburg (NL); Martijn Theodorus Lambert AKSE, TILBURG (NL)

(73) Assignee: KONINKLIJKE PHILIPS N.V., EINDHOVEN (NL)

(21) Appl. No.: 16/349,414

(22) PCT Filed: Nov. 24, 2017
(86) PCT No.: PCT/EP2017/080298

§ 371 Date: May 13, 2019

(30) Foreign Application Priority Data
Nov. 25, 2016 (EP) 16200697.7

(51) Int. Cl. C12Q 1/6886; G16B 25/10

(52) U.S. Cl. CPC C12Q 1/6886; G16B 25/10; C12Q 2600/118; C12Q 2600/112; C12Q 2600/106; C12Q 2600/158

(57) ABSTRACT

The present invention relates to certain target genes of the FOXO transcription factor family, which are markers for an oxidative stress state and can be used for inferring an oxidative stress state of a FOXO transcription factor element in the body of a medical subject. The invention further relates to methods for inferring an oxidative stress state of a FOXO transcription element and for inferring the activity of the FOXO/PI3K cellular signalling pathway based on expression levels of the target genes as well as products to perform the methods.

### FIELD OF THE INVENTION

The present invention generally relates to the field of bioinformatics, genomic/transcriptomic processing, proteomic processing, and related arts. More particularly, the present invention relates to certain target genes of the FOXO transcription factor family, which are markers for an oxidative stress state and can be used for inferring an oxidative stress state of a FOXO transcription factor element in a medical subject. The present invention also relates to a method for inferring an oxidative stress state of a FOXO transcription factor element in a medical subject based on the expression level(s) of one or more of the FOXO target genes and a method for inferring the activity of the FOXO/PI3K cellular signaling pathway in a medical subject based on expression levels of one or more target gene(s) of the FOXO/PI3K cellular signaling pathway measured in an extracted sample of the medical subject and based on the inferred oxidative stress state of the FOXO transcription factor element in the medical subject. The present invention further relates to a product comprising primers and/or probes for determining the expression levels of the FOXO target genes. The present invention further relates to an apparatus comprising a digital processor configured to perform the method, a non-transitory storage medium storing instructions that are executable by a digital processing device to perform such a method, and a computer program comprising program code means for causing a digital processing device to perform such a method.

### BACKGROUND OF THE INVENTION

Genomic/transcriptomic and proteomic analyses have substantial realized and potential promise for clinical application in medical fields such as oncology, where various cancers are known to be associated with specific combinations of genomic mutations/variations and/or high or low expression levels for specific genes, which play a role in growth and evolution of cancer, e.g., cell proliferation and metastasis.

For example, screening for an over-expression of the HER2 receptor on the membrane of cells in breast cancer samples is currently the standard test performed for identifying patients that are eligible to HER2 inhibitors such as Trastuzumab. Over-expression of the ERBB2 gene, which results in an over-expression of the HER2 receptor on the cell membrane, occurs in approximately 25% to 30% of all breast cancers and is associated with an increased disease recurrence and a poor prognosis. However, the expression of the HER2 receptor is by no means a conclusive indicator for driving tumor growth as the signaling initiated by the HER2 receptor can for instance be dampened by the downstream cellular signaling pathway. This also seems to be reflected in the initial response rate of 26% in HER2-positive breast cancer patients treated with Trastuzumab (Charles L. Vogel, et al., “Efficacy and Safety of Trastuzumab as a Single Agent in First-Line Treatment of HER2-Overexpressing Metastatic Breast Cancer”, Journal of Clinical Oncology, Vol. 20, No. 3, February 2002, pages 719 to 726). Besides that, the cellular signaling pathway downstream of the HER2 receptor can also be activated by mutations/over-expression in proteins downstream of the HER2 receptor, resulting in (a) relatively aggressive tumor type(s) that will not be detected by measuring HER2 expression levels.

It has been shown that the possibilities of characterizing patients having a tumor, e.g., breast cancer, can be improved by studying effects occurring in the cellular signaling pathway downstream of the HER2 receptor. Thus, a method for inferring activity of a PI3K cellular signaling pathway using mathematical modelling of target gene expressions has been described in the published international patent application WO 2015/101635 A1 (“Assessment of the PI3K cellular signaling pathway activity using mathematical modelling of target gene expression”).

According to WO 2015/101635 A1 the method for inferring activity of a PI3K cellular signaling pathway using mathematical modelling of target gene expressions, comprises:

inferring the activity of the FOXO/PI3K cellular signaling pathway in a medical subject based at least on expression levels of one or more target gene(s) of the FOXO/PI3K cellular signaling pathway measured in an extracted sample of the medical subject, wherein the inferring comprises:

determining an activity level of a FOXO transcription factor element in the extracted sample of the medical subject, the FOXO transcription factor element controlling transcription of the one or more target gene(s) of the PI3K cellular signaling pathway, the determining being based at least in part on evaluating a mathematical model relating expression levels of the one or more target gene(s) of the FOXO/PI3K cellular signaling pathway to the activity level of the FOXO transcription factor element;

inferring the activity of the PI3K cellular signaling pathway in the medical subject based on the determined activity level of the FOXO transcription factor element in the extracted sample of the medical subject.

In this context it has been realized that a suitable way of identifying effects occurring in the cellular signaling pathway downstream of the HER2 receptor, such as the PI3K cellular signaling pathway, can be based on a measurement of the signaling output of the cellular signaling pathway, which is—amongst others—the transcription of the target genes by a transcription factor (TF), such as a FOXO transcription factor element, controlled by the cellular signaling pathway. The PI3K cellular signaling pathway targeted herein is not only linked to breast cancer, but is known to be inappropriately activated in many types of cancer (Jeffrey A. Engelman, “Targeting PI3K signalling in cancer: opportunities, challenges and limitations”, Nature Reviews Cancer, No. 9, August 2009, pages 550 to 562). It is thought to be regulated by the RTK receptor family, which also includes the HER-family. Subsequently, the PI3K cellular signaling pathway passes on its received signal(s) via a multitude of processes, of which the two main branches are the activation of the mTOR complexes and the inactivation of a family of transcription factors often referred to as FOXO (cf. the figure showing the PI3K cellular signaling pathway in the above article from Jeffrey A. Engelman). The method concentrates on the PI3K cellular signaling pathway and the FOXO TF family, the activity of which is substantially negatively correlated with the activity of the PI3K cellular signaling pathway, i.e., activity of FOXO is substantially correlated with inactivity of the PI3K cellular signaling pathway, whereas inactivity of FOXO is substantially correlated with activity of the PI3K cellular signaling pathway. The method makes it possible to determine the activity of the PI3K cellular signaling pathway in a medical subject by (i) determining an activity level of a FOXO transcription factor element in the extracted sample of the medical subject, wherein the determining is based at least in part on evaluating a mathematical model relating expression levels of one or more target gene(s) of the PI3K cellular signaling pathway, the transcription of which is controlled by the FOXO transcription factor element, to the transcriptional activity level of the FOXO transcription factor element, and by (ii) inferring the activity of the PI3K cellular signaling pathway in the medical subject based on the determined activity level of the FOXO transcription factor element in the extracted sample of the medical subject. This allows improving the possibilities of characterizing patients that have a tumor, e.g., breast cancer, which is at least partially driven by a deregulated PI3K cellular signaling pathway, and that are therefore likely to respond to inhibitors of the PI3K cellular signaling pathway.

The nuclear FOXO3 transcription factor, a member of the FOXO transcription factor family, for example, can be active in normal cells but also in a situation in which the cell experiences oxidative stress, like in a cancer cell. In both situations FOXO is present in the nucleus (cf. FIG. 1). When the PI3K cellular signaling pathway becomes active FOXO is translocated from nucleus to cytoplasm, which is associated with inactivity of FOXO. This is not possible, however, when FOXO is activated due to oxidative stress.

Therefore, in a situation, in which FOXO is found to be active and in the nucleus, this may either be a normal cell, in which the PI3K pathway is inactive and FOXO is in a tumor suppressive state or it may be in a situation in which the cell experiences oxidative stress and in which the PI3K pathway is active but FOXO translocation from the nucleus and inactivity is prevented. It is therefore desirable to find a way to distinguish a tumor suppressive FOXO activity from oxidative stress in order to render the inference that the PI3K pathway is active or inactive more reliable. A decision tree to infer PI3K activity is shown in FIG. 2.

### SUMMARY OF THE INVENTION

In accordance with a main aspect of the present invention, the above problem is solved by a FOXO target gene or set of two or more FOXO target genes for use as marker for an oxidative stress state of a FOXO transcription factor element in a medical subject based on the expression level(s) of the one or the set of two or more FOXO target gene(s) in an extracted sample of the medical subject, wherein the target gene(s) is/are selected from the group consisting of SOD2, BNIP3, MXI1, PCK1, PPARGC1A and CAT.

The present invention is based on the realization that specific FOXO target genes are differentially expressed between the “normal”, i.e. the tumor suppressive state, and an “oxidative stress” state and therefore measuring their expression levels allows distinction between the two states.

Herein, a FOXO transcription factor (TF) element is defined to be a protein complex containing at least one of the FOXO TF family members, i.e., FOXO1, FOXO3, FOXO4 and FOXO6, which is capable of binding to specific DNA sequences, thereby controlling transcription of target genes.

An oxidative stress state of a FOXO transcription factor element herein refers to a state, in which FOXO is active and in the nucleus but wherein the PI3K pathway can be either active or inactive. If the PI3K pathway is active, deactivation of FOXO does not occur due to the cell experiencing oxidative stress. In particular, an oxidative stress state refers to a state of cancer or a pre-cancer stage of a cell.

In contrast, a tumor suppressive state of a FOXO transcription factor element herein refers to a state, in which FOXO is active in the nucleus and the PI3K pathway is inactive. In particular, a tumor suppressive state refers to a normal, healthy state of a cell.

A “PI3K cellular signaling pathway” or “PI3K pathway” herein preferably refers to a cellular signaling pathway that ultimately leads to transcriptional activity of the transcription factor (TF) complexes associated with the pathway. In the present case, these consist of a FOXO TF family member as mentioned above. The pathway may therefore also be referred to as the “FOXO/PI3K cellular signaling pathway” in the context of the present invention.

The “target gene(s)” may be “direct target genes” and/or “indirect target genes”.

Suitable target genes are described in the following text.

SOD2 (superoxide dismutase-2) is a mitochondrial matrix enzyme that scavenges oxygen radicals produced by the extensive oxidation-reduction and electron transport reactions occurring in mitochondria.

BNIP3 (BcL-2/adenovirus E1B-19-kDa protein interacting protein 3) is normally expressed as an inactive monomer, but following toxic stimuli, it forms stable homodimers, integrates into the outer mitochondrial membrane, and causes loss of mitochondrial membrane potential and cell death (Sassone et al., “BNIP3 has a key role in the mitochondrial dysfunction induced by mutant huntigtin”, Human Molecular Genetics, Vol. 24, 2015, pages 6530-6539).

PCK1 (Phosphoenolpyruvate carboxykinase) is a main target for regulation of gluconeogenesis. Transcription of the PEPCK gene is regulated by insulin, glucocorticoids, cAMP, and diet, in order to adjust glucose production to physiologic requirements.

The MXI1 gene encodes a basic helix-loop-helix leucine zipper transcription factor that bind MAX in vitro, forming a sequence-specific DNA-binding complex similar to the MYC-MAX heterodimer. MXI1 antagonizes MYC function and is a candidate tumor suppressor gene. (Delpuech O, Griffiths B, East P, Essafi A, Lam E W, Burgering B, Downward J, Schulze A. “Induction of Mxi1-SR alpha by FOXO3a contributes to repression of Myc-dependent gene expression”, Molecular Cell Biology, July 2007; Vol. 27(13), pages 4917-30)

PPARGC1A is a coactivator of nuclear receptors and other transcription factors that regulate metabolic processes, including mitochondrial biogenesis and respiration, hepatic gluconeogenesis, and muscle fiber-type switching (Lin et al., “Defects in adaptive energy metabolism with CNS-linked hyperactivity in PGC-1-alpha null mice”, Cell, 2004, Vol. 119, pages 121-135).

According to a preferred embodiment, the present invention relates to a set of at least four FOXO target genes, preferably all target genes, selected from the group consisting of SOD2, BNIP3, MXI1, PCK1, PPARGC1A and CAT for use as marker as described above.

According to a particularly preferred embodiment, the present invention relates to a set of two or more FOXO target genes, preferably all target genes, selected from the group consisting of SOD2, BNIP3, MXI1 and PCK1 for use as marker as described above.

Another aspect of the present invention relates to the use of a FOXO target gene or of a set of two or more FOXO target genes as marker for inferring an oxidative stress state of a FOXO transcription factor element in a medical subject based on the expression levels of the one or the set of two or more FOXO target gene(s) in an extracted sample of the medical subject, wherein the target gene(s) is/are selected from the group consisting of SOD2, BNIP3, MXI1, PCK1, PPARGC1A and CAT.

The term “inferring” in the context of the present invention refers to the act of applying a created mathematical expression or model to a dataset measured in a sample, such as expression levels of specific genes, to derive information relating to a state of the sample. For example, “inferring” may involve the calculation of a score, such as an oxidative stress score, for a sample and deducing a state, such as an oxidative stress state, by e.g. applying a threshold, wherein the score is either above or below the threshold according to the state of the sample.

The “subject” or “medical subject” may be a human or an animal.

The extracted sample may be a sample of the tissue and/or the cells and/or the body fluid of the medical subject or may be from a cell line and/or a tissue culture derived from a medical subject and, if applicable, cultivated in vitro in the lab (e.g., for regenerative medicine purposes). The sample can be, e.g., a sample obtained from a cancer lesion, or from a lesion suspected for cancer, or from a metastatic tumor, or from a body cavity in which fluid is present which is contaminated with cancer cells (e.g., pleural or abdominal cavity or bladder cavity), or from other body fluids containing cancer cells, and so forth, preferably via a biopsy procedure or other sample extraction procedure. The cells of which a sample is extracted may also be tumorous cells from hematologic malignancies (such as leukemia or lymphoma). In some cases, the cell sample may also be circulating tumor cells, that is, tumor cells that have entered the bloodstream and may be extracted using suitable isolation techniques, e.g., apheresis or conventional venous blood withdrawal. Aside from blood, the body fluid of which a sample is extracted may be urine, gastrointestinal contents, or an extravasate. The term “extracted sample”, as used herein, also encompasses the case where tissue and/or cells and/or body fluid of the subject have been taken from the subject and, e.g., have been put on a microscope slide, and where for performing the claimed method a portion of this sample is extracted, e.g., by means of Laser Capture Microdissection (LCM), or by scraping off the cells of interest from the slide, or by fluorescence-activated cell sorting techniques. The cells or tissue can also be from normal, non-malignant tissue, or from diseased tissue other than cancer.

Preferred is the use as described above, wherein inferring an oxidative stress state of a FOXO transcription factor element in a medical subject is based on the expression levels of at least four FOXO target genes, preferably all target genes selected from the group consisting of SOD2, BNIP3, MXI1, PCK1, PPARGC1A and CAT in an extracted sample of the medical subject.

Further preferred is the use as described above, wherein inferring an oxidative stress state of a FOXO transcription factor element in a medical subject is based on the expression levels of two or more FOXO target genes, preferably all target genes, selected from the group consisting of SOD2, BNIP3, MXI1 and PCK1, in an extracted sample of the medical subject.

According to another main aspect, the present invention relates to a method for inferring an oxidative stress state of a FOXO transcription factor element in a medical subject, wherein the inferring comprises:

determining the expression levels of one or more FOXO target gene(s) in an extracted sample of the medical subject, wherein the target gene(s) is/are selected from the group consisting of SOD2, BNIP3, MXI1, PCK1, PPARGC1A and CAT; and

inferring the oxidative stress state of the FOXO transcription factor element in the medical subject based on the determined expression levels of one or more FOXO target gene(s) in the extracted sample of the medical subject.

The distinguishing set of target genes used for determining the oxidative stress state of a FOXO transcription factor element as opposed to a tumor suppressive state was found by comparing the target gene expression profile in normal breast tissue and normal colon tissue samples in which FOXO is active, with that in samples from respectively breast cancer and colon cancer.

A preferred embodiment is a method as described above, wherein the inferring is based on the expression level(s) of at least four, preferably all of the FOXO target gene(s) selected from the group consisting of SOD2, BNIP3, MXI1, PCK1, PPARGC1A and CAT in the extracted sample of the medical subject.

A further preferred embodiment is a method as described above, wherein the inferring is based on the expression level(s) of two or more, preferably all of the FOXO target gene(s) selected from the group consisting of SOD2, BNIP3, MXI1 and PCK1 in the extracted sample of the medical subject.

The target genes recited in the paragraph above have been found to be particularly informative with respect to the oxidative stress state.

In another embodiment, the present invention relates to a method as described above, wherein an oxidative stress state of the FOXO transcription factor element is inferred, when the expression level(s) of SOD2 and/or BNIP3 are upregulated in the extracted sample of the medical subject compared to a control sample and/or when the expression level(s) of one or more target gene(s) selected from MXI1, PCK1, PPARGC1A and CAT are downregulated in the extracted sample of the medical subject compared to a control sample.

A preferred embodiment is a method as described above, wherein an oxidative stress state of the FOXO transcription factor element is inferred, when the expression level(s) of SOD2 and/or BNIP3 are upregulated in the extracted sample of the medical subject compared to a control sample and/or when the expression level(s) of MXI1 and/or PCK1 are downregulated in the extracted sample of the medical subject compared to a control sample.

As mentioned above, the distinguishing set of target genes used for determining the oxidative stress state of a FOXO transcription factor element as opposed to a tumor suppressive state was found by comparing the target gene expression profile in normal breast tissue and normal colon tissue samples in which FOXO is active, with that in samples from respectively breast cancer and colon cancer.

The results can be summarized such that a comparison of FOXO being active in normal colon versus FOXO active in colon carcinoma shows:

increased expression levels of SOD2 and BNIP3 in colon cancer, and

increased expression levels of MXI1, PCK1 and PPARGC1A in normal colon tissue.

The above results were reproducible in FOXO active breast cancer versus FOXO active in normal breast tissue:

increased expression levels of SOD2 and BNIP3 in breast cancer, and

increased expression levels of MXI1, PCK1, CAT and PPARGC1A in normal breast tissue.

The results were also at least partially reproducible in FOXO active esophageal cancer versus FOXO active in normal esophageal tissue:

increased expression level of SOD2 in esophageal cancer, and

increased expression levels of MXI1 and PPARGC1A in normal esophageal tissue.

Accordingly, the method of the invention can be used to indicate a cancer or pre-cancer state in a medical subject, in particular it can be used to determine the presence or absence of colon cancer, breast cancer and esophageal cancer.

The most distinguishing genes are SOD2, BNIP3 (both increased in cancer) and MXI1, PCK1 (both decreased in cancer). PPARGC1A is less informative in breast cancer and therefore less preferred.

The control sample can be a sample of “normal” tissue, cells or body fluid extracted from a healthy medical subject or it can refer to averaged expression data over collected samples from multiple healthy medical subjects. Such expression data may be derived from public databases.

The method according to the invention may also be a method for inferring the activity of a PI3K cellular signaling pathway in a subject based at least on the oxidative stress state of the subject, comprising,

inferring the oxidative stress state of the subject based on the expression level(s) of one or more gene(s) selected from the group consisting of SOD2, BNIP3, MXI1, PCK1, PPARGC1A and CAT in the subject.

The oxidative stress state of the subject is preferably the oxidative stress state of a FOXO transcription factor element in the subject.

The method is preferably based at least on expression levels of one or more target gene(s) of the PI3K cellular signaling pathway measured in the subject, which may be a medical subject. Preferably, the method is performed using an extracted sample of the subject, i.e. expression levels are measured in an extracted sample of the subject.

According to a further aspect of the present invention, the method described above can be integrated into a method for inferring the activity of the FOXO/PI3K cellular signalling pathway as e.g. described in WO 2015/101635 A1 and improve the reliability of the result as explained above.

Preferably, inferring the activity of the PI3K cellular signaling pathway in the subject is therefore based on the inferred oxidative stress state of the subject and the activity level of the FOXO transcription factor element in the subject.

The method may comprise determining the expression level(s) of the one or more genes in the subject.

The expression levels of the one or more gene(s) are preferably determined in an extracted sample of the subject.

Preferably, inferring the oxidative stress state of the subject is based on the expression level(s) of at least four, preferably all of the FOXO target gene(s) selected from the group consisting of SOD2, BNIP3, MXI1, PCK1, PPARGC1A and CAT in the subject.

Further preferably, inferring the oxidative stress state of the subject is based on the expression level(s) of two or more, preferably all of the FOXO target gene(s) selected from the group consisting of of SOD2, BNIP3, MXI1 and PCK1 in the subject.

Further preferably, inferring the oxidative stress state of the subject is based on the expression level of one FOXO target gene selected from the group consisting of SOD2, BNIP3, MXI1, PCK1, PPARGC1A and CAT in the subject.

Another preferred embodiment is a method as described above, further comprising inferring the activity of the FOXO/PI3K cellular signalling pathway in the medical subject based at least on expression levels of one or more target gene(s) of the FOXO/PI3K cellular signalling pathway measured in an extracted sample of the medical subject, wherein the inferring comprises:

determining an activity level of a FOXO transcription factor element in the extracted sample of the medical subject, the FOXO transcription factor element controlling transcription of the one or more target gene(s) of the FOXO/PI3K cellular signalling pathway, the determining being based at least in part on evaluating a mathematical model relating expression levels of the one or more target gene(s) of the FOXO/PI3K cellular signalling pathway to the activity level of the FOXO transcription factor element;

inferring the activity of the FOXO/PI3K cellular signalling pathway in the medical subject based on the determined activity level of the FOXO transcription factor element in the extracted sample of the medical subject and the inferred oxidative stress state of the FOXO transcription factor element in the medical subject,

wherein inferring the activity of the FOXO/PI3K cellular signaling pathway is performed by a digital processing device using the mathematical model.

A further preferred embodiment of the present invention is a method for inferring the activity of the FOXO/PI3K cellular signalling pathway in a medical subject based at least on expression levels of one or more target gene(s) of the FOXO/PI3K cellular signalling pathway measured in an extracted sample of the medical subject, wherein the inferring comprises:

determining an activity level of a FOXO transcription factor element in the extracted sample of the medical subject, the FOXO transcription factor element controlling transcription of the one or more target gene(s) of the FOXO/PI3K cellular signalling pathway, the determining being based at least in part on evaluating a mathematical model relating expression levels of the one or more target gene(s) of the FOXO/PI3K cellular signalling pathway to the activity level of the FOXO transcription factor element;

inferring the oxidative stress state of the FOXO transcription factor element in the medical subject based on the expression levels of one or more FOXO target gene(s) selected from the group consisting of SOD2, BNIP3, MXI1, PCK1, PPARGC1A and CAT in the extracted sample of the medical subject;

inferring the activity of the FOXO/PI3K cellular signalling pathway in the medical subject based on the determined activity level of the FOXO transcription factor element in the extracted sample of the medical subject and the inferred oxidative stress state of the FOXO transcription factor element in the medical subject,

wherein inferring the activity of the FOXO/PI3K cellular signaling pathway is performed by a digital processing device using the mathematical model.

By relying not only on the determined activity level of the FOXO transcription factor element but including also the inferred oxidative stress state of the FOXO transcription factor element, the inferred activity of the FOXO/PI3K cellular signalling pathway becomes more reliable as explained above.

As will be understood by a person skilled in the art, determining the expression levels of the FOXO target gene(s) for both purposes, inferring the oxidative stress state of the FOXO transcription factor element as well as determining the activity level of the FOXO transcription factor element, may be done using the same or different samples from the same medical subject and/or the same or different probes and may be based on the same or different, or partially overlapping (sets) of target genes as applicable. If one, two or more, or all of the expression level(s) of the target gene(s) is/are selected from the group consisting of SOD2, BNIP3, MXI1, PCK1, PPARGC1A and CAT are determined the result can be used for both purposes. Preferably, only one sample for the medical subject is used to determine all of the expression levels needed for the method described above.

The mathematical model may be a probabilistic model, preferably a Bayesian network model as described in WO 2015/101635 A1, based at least in part on conditional probabilities relating the FOXO transcription factor element and expression levels of the one or more target gene(s) of the PI3K cellular signaling pathway measured in the extracted sample of the tissue and/or the cells and/or the body fluid of the medical subject, or the mathematical model may be based at least in part on one or more linear combination(s) of expression levels of the one or more target gene(s) of the PI3K cellular signaling pathway measured in the extracted sample of the tissue and/or the cells and/or the body fluid of the medical subject. In particular, the inferring of the activity of the PI3K cellular signaling pathway may be performed as disclosed in the published international patent application WO 2013/011479 A2 (“Assessment of cellular signaling pathway activity using probabilistic modeling of target gene expression”) or as described in the published international patent application WO 2014/102668 (“Assessment of cellular signaling pathway activity using linear combination(s) of target gene expressions”).

According to a preferred embodiment of the present invention, an oxidative stress state is inferred, when the expression level(s) of SOD2 and/or BNIP3 are upregulated in the extracted sample of the medical subject compared to a control sample and/or when the expression level(s) of one or more target gene(s) selected from MXI1, PCK1, PPARGC1A and CAT are downregulated in the extracted sample of the medical subject compared to a control sample.

According to a further preferred embodiment of the present invention, an oxidative stress state is inferred, when the expression level(s) of SOD2 and/or BNIP3 are upregulated in the extracted sample of the medical subject compared to a control sample and/or when the expression level(s) of MXI1 and/or PCK1 are downregulated in the extracted sample of the medical subject compared to a control sample.

Preferably, the oxidative stress state is the oxidative stress state of a FOXO transcription factor element in the subject.

According to a preferred embodiment of the present invention, the target gene(s) for inferring the activity of the PI3K cellular signaling pathway is/are selected from the group consisting of the target genes below.

In a preferred method as described above, the activity level of the FOXO transcription factor element in the subject is determined based at least on expression levels of one or more, preferably at least three, target gene(s) of the PI3K cellular signaling pathway measured in an extracted sample of the subject selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10.

In a further preferred method as described above, the activity level of the FOXO transcription factor element is determined based at least on expression levels of one or more, preferably at least three, target gene(s) of the PI3K cellular signaling pathway measured in the extracted sample of the subject selected from the group consisting of: ATP8A1, C10orf10, CBLB, DDB1, DYRK2, ERBB3, EREG, EXT1, FGFR2, IGF1R, IGFBP1, IGFBP3, LGMN, PPM1D, SEMA3C, SEPP1, SESN1, SLC5A3, SMAD4 and TLE4 and/or selected from the group consisting of: ATG14, BIRC5, IGFBP1, KLF2, KLF4, MYOD1, PDK4, RAG1, RAG2, SESN1, SIRT1, STK11 and TXNIP.

The activity level of the FOXO transcription factor element is preferably inferred based on expression level(s) of the target gene(s) recited above measured in an extracted sample of the subject.

Particularly preferred is a method wherein

inferring the activity of the FOXO/PI3K cellular signaling pathway in the medical subject is based at least on expression levels of one or more, preferably at least three, target gene(s) of the FOXO/PI3K cellular signaling pathway measured in the extracted sample of the medical subject selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10 and/or wherein inferring the oxidative stress state of the FOXO transcription factor element is based on the expression levels of one or more, preferably all of the target genes of a FOXO transcription factor SOD2, BNIP3, MXI1 and PCK1 measured in the extracted sample of the medical subject.

Further preferred is an embodiment, wherein the inferring the activity of the FOXO/PI3K cellular signaling pathway in the medical subject is based at least on expression levels of six or more, preferably ten or more, more preferably all target gene(s) of the FOXO/PI3K cellular signaling pathway measured in the extracted sample of the medical subject selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10.

Further preferred is a method, wherein the inferring is further based on expression levels of at least one target gene of the PI3K cellular signaling pathway measured in the extracted sample of the tissue and/or the cells and/or the body fluid of the medical subject selected from the group consisting of: ATP8A1, C10orf10, CBLB, DDB1, DYRK2, ERBB3, EREG, EXT1, FGFR2, IGF1R, IGFBP1, IGFBP3, LGMN, PPM1D, SEMA3C, SEPP1, SESN1, SLC5A3, SMAD4 and TLE4.

Further preferred is a method, wherein the inferring is further based on expression levels of at least one target gene of the PI3K cellular signaling pathway measured in the extracted sample of the tissue and/or the cells and/or the body fluid of the medical subject selected from the group consisting of: ATG14, BIRC5, IGFBP1, KLF2, KLF4, MYOD1, PDK4, RAG1, RAG2, SESN1, SIRT1, STK11 and TXNIP.

If the inferring is further based both on expression levels of at least one target gene selected from the group specified in the preceding paragraph and on expression levels of at least one target gene selected from the group specified in the paragraph preceding the preceding paragraph, the target gene IGFBP1, which is mentioned above with respect to both groups, may only be contained in one of the groups.

Another aspect of the present invention relates to a method (as described herein), further comprising:

determining whether the PI3K cellular signaling pathway is operating abnormally in the tissue and/or the cells and/or the body fluid of the medical subject based on the inferred activity of the PI3K cellular signaling pathway in the tissue and/or the cells and/or the body fluid of the medical subject.

In a preferred embodiment, the method as described above therefore further comprises determining whether the PI3K cellular signalling pathway is operating abnormally in the subject based on the inferred activity of the PI3K cellular signalling pathway in the subject.

The phrase “the cellular signaling pathway is operating abnormally” refers to the case where the “activity” of the pathway is not as expected, wherein the term “activity” may refer to the activity of the transcription factor complex in driving the target genes to expression. “Normal” may be when it is inactive in tissue where it is expected to be inactive and active where it is expected to be active. Furthermore, there may be a certain level of activity that is considered normal, and anything higher or lower may be considered abnormal.

The present invention also relates to a method (as described herein) further comprising:

recommending prescribing a drug for the medical subject that corrects for abnormal operation of the PI3K cellular signaling pathway,

wherein the recommending is performed only if the PI3K cellular signaling pathway is determined to be operating abnormally in the tissue and/or the cells and/or the body fluid of the medical subject based on the inferred activity of the PI3K cellular signaling pathway.

According to a preferred embodiment the method of the present invention therefore further comprises recommending prescribing a drug for the subject that corrects for abnormal operation of the PI3K cellular signalling pathway, wherein the recommending is performed if the PI3K cellular signalling pathway is determined to be operating abnormally in the subject based on the inferred activity of the PI3K cellular signalling pathway.

Advantageously, the method as described above can be used to indicate a cancer or pre-cancer state in a medical subject, in particular it can be used to determine the presence or absence of colon cancer, breast cancer and esophageal cancer.

The present invention also relates to a method (as described herein), wherein the inferring comprises:

inferring the activity of the PI3K cellular signaling pathway in the medical subject based at least on expression levels of two, three or more target genes of a set of target genes of the PI3K cellular signaling pathway measured in the extracted sample of the tissue and/or the cells and/or the body fluid of the medical subject.

Preferably,

the set of target genes of the PI3K cellular signaling pathway includes at least nine, preferably all target genes selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10.

A method, wherein

the set of target genes of the PI3K cellular signaling pathway further includes at least one target gene selected from the group consisting of: ATP8A1, C10orf10, CBLB, DDB1, DYRK2, ERBB3, EREG, EXT1, FGFR2, IGF1R, IGFBP1, IGFBP3, LGMN, PPM1D, SEMA3C, SEPP1, SESN1, SLC5A3, SMAD4 and TLE4,

is particularly preferred.

A method, wherein

the set of target genes of the PI3K cellular signaling pathway further includes at least one target gene selected from the group consisting of: ATG14, BIRC5, IGFBP1, KLF2, KLF4, MYOD1, PDK4, RAG1, RAG2, SESN1, SIRT1, STK11 and TXNIP,

is also particularly preferred.

If the set of target genes further includes both at least one target gene selected from the group specified in the preceding paragraph and at least one target gene selected from the group specified in the paragraph preceding the preceding paragraph, the target gene IGFBP1, which is mentioned above with respect to both groups, may only be contained in one of the groups.

In a further aspect, the present invention also relates to a product comprising:

primers and/or probes for determining a gene expression level of one or a set of two or more FOXO target gene(s), preferably at least four FOXO target genes in an extracted sample of a medical subject, wherein the target gene(s) is/are selected from the group consisting of SOD2, BNIP3, MXI1, PCK1, PPARGC1A and CAT, preferably selected from SOD2, BNIP3, MXI1 and PCK1; and

optionally further comprising primers and/or probes for determining the expression levels of a set of genes other than the above mentioned genes in the extracted sample of a medical subject, preferably of two or more target gene(s) of the FOXO/PI3K cellular signaling pathway selected from the group consisting of: AGRP, BCL2L11, BCL6, BTG1, CAV1, CCND1, CCND2, CCNG2, CDKN1A, CDKN1B, ESR1, FASLG, FBXO32, GADD45A, INSR, NOS3, POMC, PRDX3, RBL2 and TNFSF10.

In a preferred embodiment, the product described above is a PCR kit, a RNA-sequencing kit, or a microarray kit.

The materials for use in the methods of the present invention are ideally suited for preparation of kits produced in accordance with well known procedures. The invention thus provides kits comprising agents for the detection of expression of the disclosed genes and sequences. Such kits optionally comprise the agent with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, RGTP and UTP), reverse transcriptase, DNA polymerase, RNA polymerase, and one or more primers. A set of instructions will also typically be included.

In the context of the present invention, the expression level(s) may be determined by a method involving the detection of an mRNA encoded by the gene.

For example, the measurement of the nucleic acid level of marker gene(s) expression may be assessed by purification of nucleic acid molecules (e.g. RNA or cDNA) obtained from the sample, followed by hybridization with specific oligonucleotide probes as defined herein above. Comparison of expression levels may be accomplished visually or by means of an appropriate device. Methods for the detection of mRNA or expression products are known to the person skilled in the art.

Alternatively, the nucleic acid level of marker gene(s) expression may be detected in a DNA array or microarray approach. Typically, sample nucleic acids derived from patients to be tested are processed and labeled, preferably with a fluorescent label. Subsequently, such nucleic acid molecules may be used in a hybridization approach with immobilized capture probes corresponding to the marker genes of the present invention. Suitable means for carrying out microarray analyses are known to the person skilled in the art.

In a standard setup a DNA array or microarray comprises immobilized high-density probes to detect a number of genes. The probes on the array are complementary to one or more parts of the sequence of the marker genes. Typically, cDNAs, PCR products, and oligonucleotides are useful as probes.

A DNA array- or microarray-based detection method typically comprises the following steps: (1) Isolating mRNA from a sample and optionally converting the mRNA to cDNA, and subsequently labeling this RNA or cDNA. Methods for isolating RNA, converting it into cDNA and for labeling nucleic acids are described in manuals for micro array technology. (2) Hybridizing the nucleic acids from step 1 with probes for the marker genes. The nucleic acids from a sample can be labeled with a dye, such as the fluorescent dyes Cy3 (red) or Cy5 (blue). Generally, a control sample is labeled with a different dye. (3) Detecting the hybridization of the nucleic acids from the sample with the probes and determining at least qualitatively, and more particularly quantitatively, the amounts of mRNA in the sample for marker genes investigated. The difference in the expression level between sample and control can be estimated based on a difference in the signal intensity. These can be measured and analyzed by appropriate software such as, but not limited to the software provided for example by Affymetrix.

There is no limitation on the number of probes corresponding to the marker genes used, which are spotted on a DNA array. Also, a marker gene can be represented by two or more probes, the probes hybridizing to different parts of a gene. Probes are designed for each selected marker gene. Such a probe is typically an oligonucleotide comprising 5-50 nucleotide residues. Longer DNAs can be synthesized by PCR or chemically. Methods for synthesizing such oligonucleotides and applying them on a substrate are well known in the field of micro-arrays. Genes other than the marker genes may be also spotted on the DNA array. For example, a probe for a gene whose expression level is not significantly altered may be spotted on the DNA array to normalize assay results or to compare assay results of multiple arrays or different assays.

Alternatively, the nucleic acid level of marker gene(s) expression may be detected in a quantitative RT-PCR approach, preferably in a real-time PCR approach following the reverse transcription transcripts of interest. Typically, as first step, a transcript is reverse transcribed into a cDNA molecule according to any suitable method known to the person skilled in the art. A quantitative or real-time PCR approach may subsequently be carried out based on a first DNA strand obtained as described above.

Preferably, Taqman or Molecular Beacon probes as principal FRET-based probes of this type may be used for quantitative PCR detection. In both cases, the probes, serve as internal probes which are used in conjunction with a pair of opposing primers that flank the target region of interest, preferably a set of marker gene(s) specific oligonucleotides as defined herein above. Upon amplification of a target segment, the probe may selectively bind to the products at an identifying sequence in between the primer sites, thereby causing increases in FRET signaling relative to increases in target frequency.

Preferably, a Taqman probe to be used for a quantitative PCR approach according to the present invention may comprises a specific oligonucleotide as defined above of about 22 to 30 bases that is labeled on both ends with a FRET pair. Typically, the 5′ end will have a shorter wavelength fluorophore such as fluorescein (e.g. FAM) and the 3′ end is commonly labeled with a longer wavelength fluorescent quencher (e.g. TAMRA) or a non-fluorescent quencher compound (e.g. Black Hole Quencher). It is preferred that the probes to be used for quantitative PCR, in particular probes as defined herein above, have no guanine (G) at the 5′ end adjacent to the reporter dye in order to avoid quenching of the reporter fluorescence after the probe is degraded.

A Molecular Beacon probe to be used for a quantitative PCR approach according to the present invention preferably uses FRET interactions to detect and quantify a PCR product, with each probe having a 5′ fluorescent-labeled end and a 3′ quencher-labeled end. This hairpin or stem-loop configuration of the probe structure comprises preferably a stem with two short self-binding ends and a loop with a long internal target-specific region of about 20 to 30 bases.

Alternative detection mechanisms which may also be employed in the context of the present invention are directed to a probe fabricated with only a loop structure and without a short complementary stem region. An alternative FRET-based approach for quantitative PCR which may also be used in the context of the present invention is based on the use of two hybridization probes that bind to adjacent sites on the target wherein the first probe has a fluorescent donor label at the 3 ‘ end and the second probe has a fluorescent acceptor label at its 5’ end.

In accordance with another disclosed aspect, an apparatus comprises a digital processor configured to perform a method according to the present invention as described herein.

In accordance with another disclosed aspect, a non-transitory storage medium stores instructions that are executable by a digital processing device to perform a method according to the present invention as described herein. The non-transitory storage medium may be a computer-readable storage medium, such as a hard drive or other magnetic storage medium, an optical disk or other optical storage medium, a random access memory (RAM), read only memory (ROM), flash memory, or other electronic storage medium, a network server, or so forth. The digital processing device may be a handheld device (e.g., a personal data assistant or smartphone), a notebook computer, a desktop computer, a tablet computer or device, a remote network server, or so forth.

In accordance with another disclosed aspect, a computer program comprises program code means for causing a digital processing device to perform a method according to the present invention as described herein. The digital processing device may be a handheld device (e.g., a personal data assistant or smartphone), a notebook computer, a desktop computer, a tablet computer or device, a remote network server, or so forth.

The present invention as described herein can, e.g., also advantageously be used in connection with:

diagnosis based on the inferred activity of the PI3K cellular signaling pathway in the tissue and/or the cells and/or the body fluid of the medical subject;

prognosis based on the inferred activity of the PI3K cellular signaling pathway in the tissue and/or the cells and/or the body fluid of the medical subject;

drug prescription based on the inferred activity of the PI3K cellular signaling pathway in the tissue and/or the cells and/or the body fluid of the medical subject;

prediction of drug efficacy based on the inferred activity of the PI3K cellular signaling pathway in the tissue and/or the cells and/or the body fluid of the medical subject;

prediction of adverse effects based on the inferred activity of the PI3K cellular signaling pathway in the tissue and/or the cells and/or the body fluid of the medical subject;

monitoring of drug efficacy;

drug development;

assay development;

pathway research;

cancer staging;

enrollment of the medical subject in a clinical trial based on the inferred activity of the PI3K cellular signaling pathway in the tissue and/or the cells and/or the body fluid of the medical subject;

selection of subsequent test to be performed; and

selection of companion diagnostics tests.

According to a preferred embodiment, the method of the present invention is therefore used in at least one of the following activities:

diagnosis based on the inferred activity of the PI3K cellular signalling pathway in the subject;

prognosis based on the inferred activity of the PI3K cellular signalling pathway in the subject;

drug prescription based on the inferred activity of the PI3K cellular signalling pathway in the subject;

prediction of drug efficacy based on the inferred activity of the PI3K cellular signalling pathway in the subject;

prediction of adverse effects based on the inferred activity of the PI3K cellular signalling pathway in the subject;

monitoring of drug efficacy;

drug development;

assay development;

pathway research;

cancer staging;

enrollment of the subject in a clinical trial based on the inferred activity of the PI3K cellular signalling pathway in the subject;

selection of subsequent test to be performed; and

selection of companion diagnostics tests.

Further advantages will be apparent to those of ordinary skill in the art upon reading and understanding the attached figures, the following description and, in particular, upon reading the detailed examples provided herein below.

It shall be understood that the method, the apparatus, the non-transitory storage medium, and the computer program of the present invention have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

FIG. 1 schematically shows the FOXO/PI3K cellular signaling pathway in a cell, wherein FOXO3 is located in the nucleus.

FIG. 2 shows a schematic decision tree for inferring activity of the PI3K cellular signalling pathway.

FIG. 3 shows the oxidative stress score for each subgroup specified in the dataset using all genes and probesets shown in table 1.

FIG. 4 shows the oxidative stress score for only those samples that had FOXO activity as inferred by the FOXO activity model described in WO 2015/101635 A1

FIG. 5 shows the oxidative stress score obtained by using only the most informative oxidative stress-induced FOXO target genes SOD2, MXI1, PCK1 and BNIP3.

FIG. 6 shows a schematic FOXO model structure incorporating oxidative stress by adding an oxidative stress node. Nodes representing probesets are not shown for readability purposes.

FIG. 7 shows the results of the model described in example 2 and shown in FIG. 6 tested on the dataset GS20916. The x-axis shows the probability of the transcription complex (TC) node to be active and the y-axis the probability of the OXI (oxidative stress state) node to be active. Black circles represent normal colon samples, empty circles represent adenocarcinoma samples and crosses represent colon carcinoma samples.

FIG. 8 shows a Bayesian computational model predicting FOXO activity. A. The Bayesian network structure used as a basis for the modeling approach shown as a simplified model of the transcriptional program of a cellular signal transduction pathway, consisting of three types of nodes: transcription factor, target gene and microarray probe sets corresponding to target genes. B. Training of the computational FOXO3 model on a public GEO dataset GSE16573, consisting of Affymetrix microarray 2.0 Plus expression data from HUVEC containing a 4OHT-inducible FOXO3.A3-ER expression construct. Each bar represents a sample analysis result. The vertical axis indicates the probability that FOXO is “active” (values above the horizontal axis) versus “inactive” (values below the horizontal axis).

FIG. 9 shows the FOXO and PI3K activity are correctly predicted in breast cancer cell lines A. Western blot analysis of FOXO3 expression levels in MCF7-FOXO3.A3 and MDA-MB-231 cells cultured in the absence or presence of doxycycline (dox) for 16 hours. The lower FOXO3 blot represents a longer exposure of the same blot. B. Biological validation of the computational FOXO3 model using MCF7-FOXO3.A3 cells treated with 20% FBS, PI3K inhibitor LY294002, doxycycline or a combination of doxycycline and LY294002 for 16 hours. Each bar represents the analysis results of one sample. The vertical axis indicates the probability that the FOXO3 is “active” (values above the horizontal axis) versus “inactive” (values below the horizontal axis. C. Biological validation of the computational FOXO3 model using MCF7-FOXO3.A3 and MDA-MB-231 cells treated with doxycycline for 16 hours. Each bar represents the analysis results of one sample. The vertical axis indicates the probability that the FOXO3 is “active” (values above the horizontal axis) versus “inactive” (values below the horizontal axis).

FIG. 10 shows targeted drugs which inhibit the PI3K pathway and induce FOXO activity. Public datasets from samples that were treated with drugs targeting growth factor pathways. FOXO activity score is indicated as log 2odds. p values resulting from of Wilcoxon rank statistical tests are indicated in the figures. A. GEO GSE51212. The lung cancer cell line HCC827 was treated either with vehicle (DMSO) or with erlotinib, AZD6244 (selumetinib) or BEZ235, as indicated (from left to right). Erlotinib inhibits EGFR; selumetinib inhibits specifically MEK1/MEK2; BEZ235 is a dual inhibitor of PI3K/mTOR. B. GEO GSE30516 dataset. Three breast cancer cell lines representing triple-negative (BT20), ER positive (MCF7), and HER2 positive breast cancer (MDA-MB-453) were treated with Erlotinib (time periods indicated, from left to right).

FIG. 11 shows the FOXO activity and cellular localisation in colon, colon adenoma and colon carcinoma A. Biological validation of the computational FOXO model in corresponding normal colon and colon adenoma patient samples within the GSE8671 dataset. Each bar represents the analysis result of one sample. The vertical axis indicates the probability that the FOXO3 is “active” (values above the horizontal axis) versus “inactive” (values below the horizontal axis). The bars on the left represent normal tissue, the bars on the right represent adenoma samples. B. Biological validation of the computational FOXO3 model using a public dataset (GSE20916) with samples of normal colon tissue (“normal colon, dis mucosa”: normal mucosa micro-dissected from tumor tissue; “distant colon, dis mucosa”: from distant healthy tissue; “normal colon, dis crypt”: micro-dissected normal crypt from tumor tissue; “distant colon, dis crypt”: microdissected crypt from distant healthy colon; “colon, surgery”: full thickness tissue from normal colon), colon adenoma (separated in micro-dissected mucosa, micro-dissected crypt and complete surgical samples) and carcinoma (separated in micro-dissected mucosa, micro-dissected crypt and complete surgical samples) patient samples. Each bar represents the analysis results of one sample. The vertical axis indicates the probability that the FOXO3 is “active” (values above the horizontal axis) versus “inactive” (values below the horizontal axis). The bars on the left represent normal tissue, the bars in the middle represent adenoma samples, and the bars on the right represent colon carcinoma samples. C. Immunohistochemical staining of FOXO3 and haematoxylin in normal colon, colon adenoma and two carcinoma patient samples. The lower panel is a magnification of the area indicated by the black box.

FIG. 12 shows the predicted FOXO activity and distinction between tumor suppressive and oxidative stress mode of FOXO activity. To obtain larger sample numbers for analysis multiple public Affymetrix datasets from the GEO database were compiled and analysed (Colon: GSE14333, GSE20916, GSE2109, GSE37364, GSE39084, GSE40967, GSE4183, GSE8671.Breast: EMTAB365, GSE10780, GSE12276, GSE18146, GSE21653, GSE26910, GSE42568, GSE45827, GSE6532, GSE7307, GSE20685, GSE9195, GSE17907. Prostate: GSE2109, GSE32982, GSE3325, GSE46602; GSE55945, GSE7307). For each analysed sample FOXO activity is shown as Log 2odds. Although a continuous scale, FOXO is in principle considered active when the FOXO activity score is higher than 0. FOXO-active samples in solid black have a SOD2 expression level that is higher than the mean+2SD of SOD expression in FOXO-active normal healthy tissue samples, indicating high likelihood of oxidative stress-induced FOXO activity. Circles indicate FOXO-active samples in which SOD2 expression is in the normal range, indicating that FOXO is likely to be active in the tumor suppressive mode. The number of PI3K active sample represents the number of FOXO inactive samples plus the number of SOD2-high samples which are also FOXO active. For detailed explanation see text. A. Colon cancer. B. Breast cancer. For details on uniform breast cancer subtyping according to Perou, see Methods in Example 4. C. Prostate cancer.

FIG. 13 shows the PI3K-FOXO pathway and distinction between tumor suppressive and oxidative stress mode of FOXO activity A. PI3K-FOXO pathway and relation with SOD2 target gene expression. In healthy normal tissue FOXO induces transcription of target genes which control cell division. When the PI3K pathway is activated, either by genomic mutations or by stimuli from the microenvironment, FOXO activity is blocked, control over cell division is lost and cell metabolism increases, associated with oxidative stress. Oxidative stress induces activation of FOXO, now with the alternative function to protect against the consequences of the stress situation in the cell. The FOXO target gene expression profile is slightly changed to include now also SOD2. B. Decision tree to decide on PI3K activity in a tissue sample. This simplified decision tree is valid, on the assumptions: (1) FOXO is expressed in the cancer cells and (2) limited contamination with healthy FOXO-active cells.

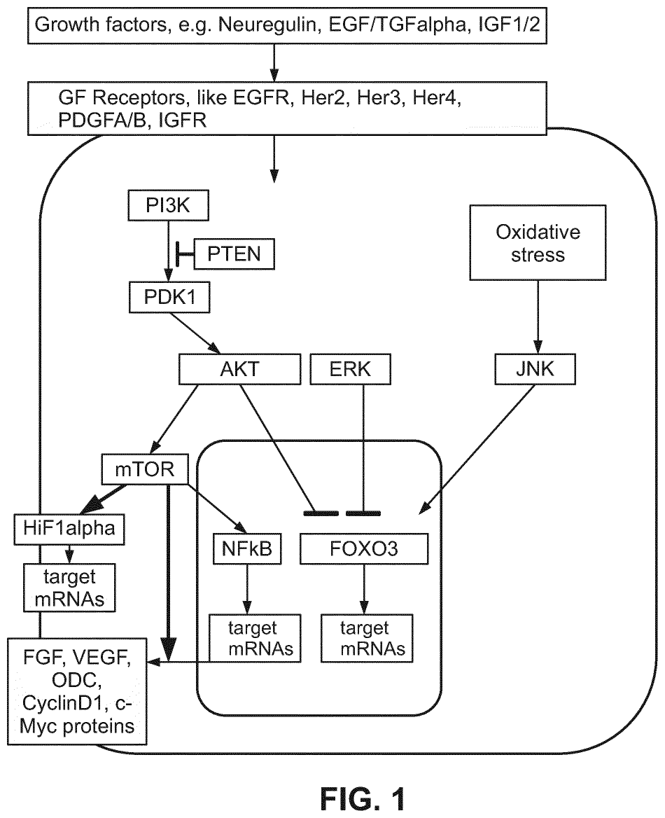

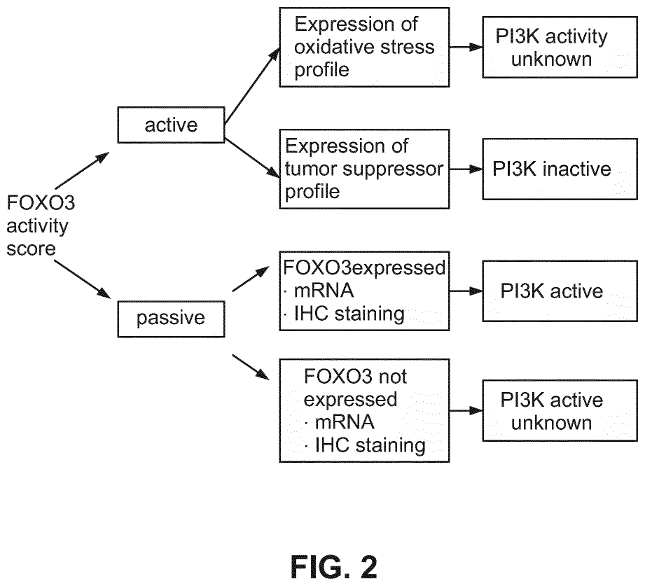

### DETAILED DESCRIPTION OF THE EMBODIMENTS

The following examples merely illustrate particularly preferred methods and selected aspects in connection therewith. The teaching provided therein may be used for constructing several tests and/or kits, e.g., to detect, predict and/or diagnose the abnormal activity of one or more cellular signaling pathways. Furthermore, upon using methods as described herein drug prescription can advantageously be guided, drug prediction and monitoring of drug efficacy (and/or adverse effects) can be made, drug resistance can be predicted and monitored, e.g., to select subsequent test(s) to be performed (like a companion diagnostic test). The following examples are not to be construed as limiting the scope of the present invention.

### Example 1: Exemplary Embodiment Using a Linear Score to Infer an Oxidative Stress State of a FOXO Transcription Factor Element

A simple linear score based on the FOXO target genes SOD2, BNIP3, PCK1, MXI1, PPARGC1A and CAT is presented here. Each target gene's expression, in this example for each probeset associated to the aforementioned genes, is compared to its expression in healthy samples for which the FOXO pathway is known to be active in its tumor-suppressive form and not in its tumor-promoting form.

In this example a threshold is defined as the average of the expression levels in healthy tissue samples plus three times the standard deviation, alternatively one can use two times the standard deviation or any other positive number, in case the gene is upregulated as a result of oxidative stress-induced activity of the FOXO pathway, which is the case for SOD2 and BNIP3. In case the gene is upregulated in the normal FOXO activity, which is the case for PCK1, MXI1, PPARGC1A and CAT, the threshold is set at the average minus three times the standard deviation of the expression in the healthy samples. Then a score is calculated for each sample by adding a point for every expression for which the expression exceeds or is lower than the set threshold for the genes upregulated by oxidative stress and upregulated in the tumor-suppressive FOXO activity, respectively.

Here the results of this score using a publically available dataset of colon samples, GSE20916, containing healthy colon samples depicted as normal colon samples, adeno(carcinomas) and colon carcinomas are shown using all genes and probesets shown in Table 1.

TABLE 1
oxidative stress-induced FOXO target genes

| | |
|---|---|
| SOD2 | 1566342_at |
| SOD2 | 215078_at |
| SOD2 | 215223_s_at |
| SOD2 | 216841_s_at |
| SOD2 | 221477_s_at |
| MXI1 | 202364_at |
| PCK1 | 208383_s_at |
| BNIP3 | 201848_s_at |
| BNIP3 | 201849_at |
| CAT | 201432_at |
| CAT | 211922_s_at |
| CAT | 215573_at |
| PPARGC1A | 1569141_a_at |
| PPARGC1A | 219195_at |

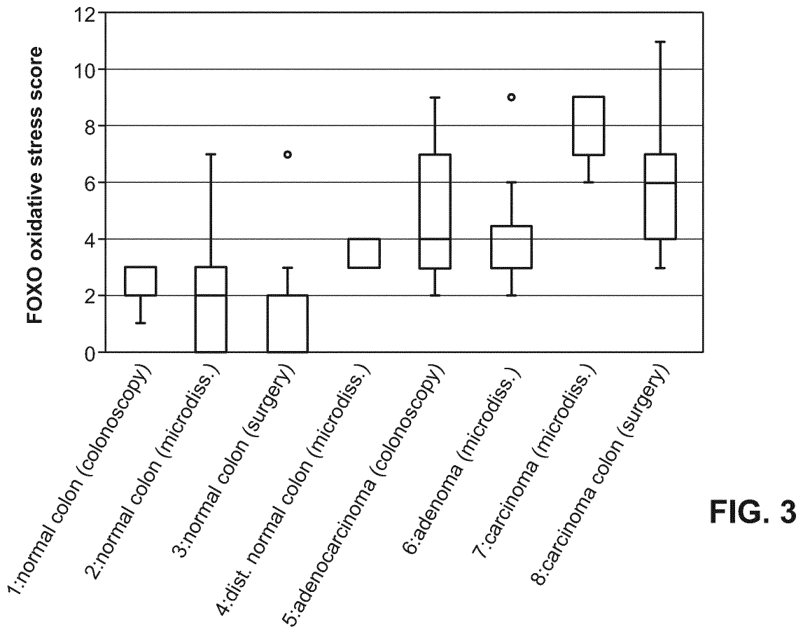

The score for each subgroup specified in GSE20916 are shown in FIG. 3. As expected the scores of the carcinoma samples are higher than the normal (healthy) samples, which is an indication that one can detect tumor-promoting FOXO activity in colon carcinoma samples (group 7 and 8) using the expression levels of the aforementioned genes as their FOXO oxidative stress scores are clearly higher than the scores in the healthy colon samples (groups 1-4). In contrast, many of the adenoma samples (group 5 and 6) also have higher FOXO oxidative stress scores compared to normal colon tissue as is evident by the higher FOXO oxidative stress score.

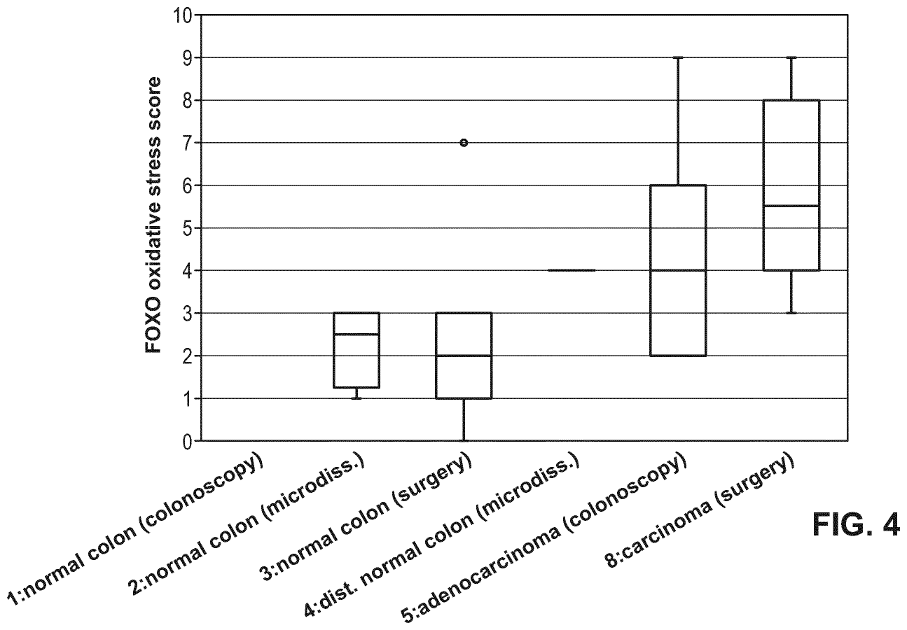

FIG. 4 depicts the FOXO oxidative stress score for only those samples from GSE20916 that had FOXO activity determined using the FOXO activity model described in WO 2015/101635 A1. Also in this case the colon carcinoma samples (group 7 and 8) have a higher FOXO oxidative stress score compared to the vast majority of the normal or healthy colon samples (groups 1-4). Also here the adenocarcinoma samples (group 5) seem to lie in between the normal and colon carcinoma samples, however only one-third of the adenocarcinoma samples were found to have an active normal FOXO TF element.

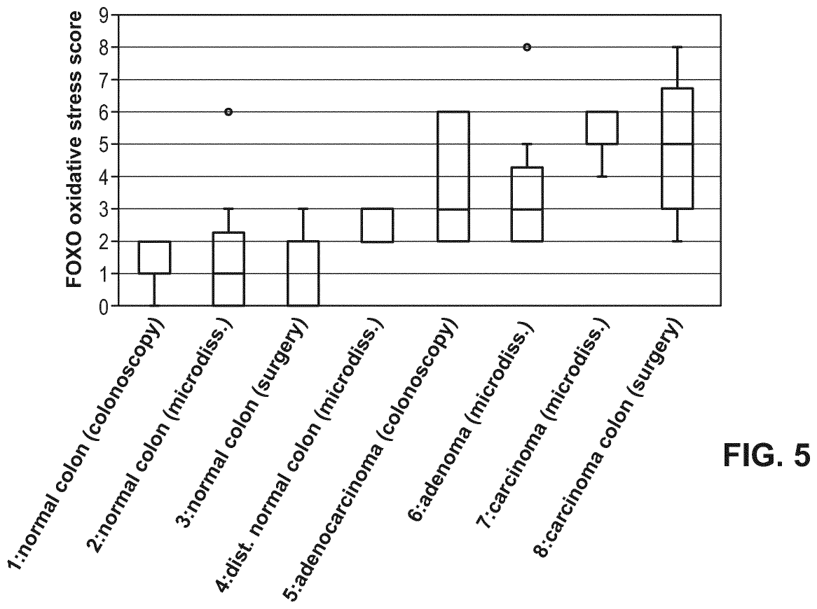

Results obtained by using only the most informative oxidative stress-induced FOXO target genes SOD2, MXI1, PCK1 and BNIP3 for all samples from GSE20916 are shown in FIG. 5. A similar behavior is observed.

As an alternative to this discrete score based on the actual measured gene expression levels on a continuous scale an alternative transformations of the expression value is proposed:
“z-score”, i.e. continuous expression levels scaled such that the average across all samples is 0 and the standard deviation is 1,
“fuzzy”, i.e. the continuous expression levels are converted to values between 0 and 1 using a sigmoid function of the following format: 1/(1+exp((thr−expr)/se)), with expr being the continuous expression levels, thr being the threshold as mentioned before and se being a softening parameter influencing the difference between 0 and 1.

### Example 2: Exemplary Embodiment Improving the Bayesian Network Described in WO 2015/101635 A1

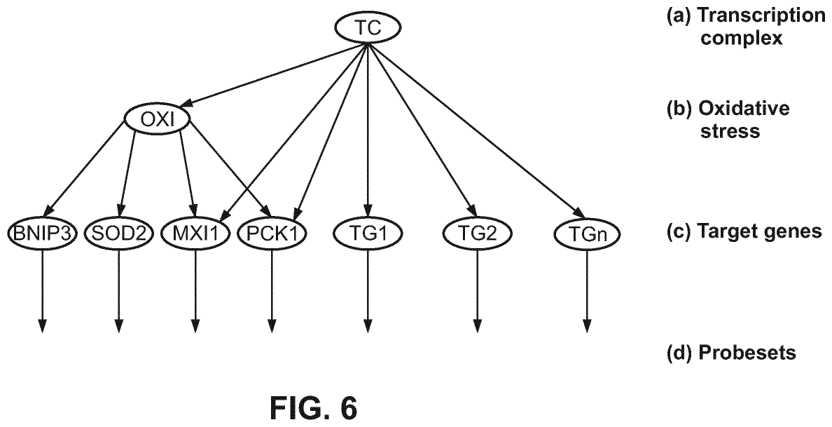

As an alternative method to model oxidative stress in the FOXO pathway, the Bayesian network described in WO 2015/101635 A1 was improved to include a separate module that represents the oxidative stress-induced FOXO activity. The structure of the Bayesian network remains the same, except for the fact that an extra node is included that defines the oxidative stress state. This node is referred to as OXI and it has two states: it is either active or inactive. FIG. 6 schematically represents the FOXO model structure incorporating oxidative stress by adding an oxidative stress node. Nodes representing probesets have not been shown for readability purposes; Table 1 shows the probesets associated with BNIP3, SOD2, MXI1 and PCK1. The probesets associated to the target genes only connected to the TC (transcription complex) node are in this example embodiment the same as described in WO 2015/101635 A1. As can be seen in FIG. 6, there is a directed edge between the FOXO pathway and oxidative stress. This allows to incorporate the knowledge that it is not possible to have an inactive FOXO pathway and at the same time oxidative stress.

As defined earlier, there are four genes (SOD2, BNIP3, PCK1 and MXI1) that are most informative indicators for oxidative stress-induced FOXO activity. Therefore, between the node OXI and genes SOD2, BNIP3, PCK1 and MXI1 directed edges can be found. Genes SOD2 and BNIP3 have higher expression levels in case there is oxidative stress, which is why there is only a directed edge from OXI and not TC. The genes PCK1 and MXI1 are affected by both the oxidative stress FOXO and the ‘normal’ FOXO nodes. Therefore, these genes have directed edges from both the TC node and the OXI node. The rest of the genes in this model are affected by the FOXO pathway only and for that reason there are only directed edges between the TC state and the rest of the target genes. Here a Bayesian network with these four FOXO oxidative stress genes is shown but a person skilled in the field can easily extend this to include the remaining two genes, PPARGC1A and CAT, as well.

As can be seen in WO 2015/101635 A1, all nodes in the network have to be quantified by means of conditional probability tables (CPT) to allow for quantitative reasoning. The CPTs of the genes and the calibration of probesets as shown in Table 1 need to be altered slightly due to the addition of the oxidative stress node. In WO 2015/101635 A1 the CPT of the edges between the probesets and target gene nodes were calibrated on samples where the pathway activity is known, but now samples known to have oxidative stress-induced FOXO activity need to be used as well. The reason for this is that a distinction in expression levels for the genes affected by oxidative stress needs to be made. Therefore, probesets of genes affected by the oxidative stress node only are calibrated using samples where the pathway is known to be switched off as inactive samples and samples with oxidative stress as active ones. For the probesets of PCK1 and MXI1 the information that they have higher expression levels in samples where FOXO is active, but only in the situation, where oxidative stress is not the cause of FOXO activity, is used. Therefore, samples that are known to have oxidative stress-induced FOXO activity are chosen as inactive samples for these probesets while the normal active FOXO samples (no oxidative stress) are chosen as active ones for these probesets. For probesets belonging to genes affected by the FOXO TC node only the calibration remains the same. The CPTs between the TC and target genes and the genes that have edges from the OXI state only are the same as in WO 2015/101635 A1. The newly defined CPT between the TC and OXI is shown in Table 2. This table reflects the knowledge that oxidative stress is unlikely with an absent TC, while no prior knowledge regarding oxidative stress in case of a present TC is available. Another CPT needs to be defined for the genes that have directed edges from both the TC and the OXI node. As both TC and OXI have two states, they combine for a total of four possibilities, so this table has eight entries. This table, defined for PCK1 and MXI1 in this case, is shown in Table 3.

TABLE 2
conditional probabilities for P[OXI|TC].

| P[OXI\|TC] | OXI = inactive | OXI = active |
|---|---|---|
| TC = absent | 0.95 | 0.05 |
| TC = present | 0.5 | 0.5 |

TABLE 3
conditional probabilities for the target genes affected
by both TC and OXI; P[TG|TC, OXI].

| | OXI = | | OXI = | |
|---|---|---|---|---|
| P[TG\|OXI, | ‘inactive’ | | ‘active’ | |
| TC] | TG = ‘down’ | TG = ‘up’ | TG = ‘down’ | TG = ‘up’ |
| TC = ‘absent’ | 0.95 | 0.05 | 0.95 | 0.05 |
| TC = | 0.3 | 0.7 | 0.95 | 0.05 |
| ‘present’ | | | | |

In the following, samples from the data set GSE20916 are used to calibrate such a Bayesian network. This data set contains normal, adenoma, adenocarcinoma and carcinoma colon samples. These samples allow to calibrate the network since experimental evidence suggests that in normal colon samples the FOXO pathway is active, but that there is no oxidative stress. We choose the samples GSM523290, GSM523314, GSM523289 and GSM523310 because these normal colon samples are predicted most active by the FOXO model of WO 2015/101635 A1. The oxidative stress state FOXO calibration samples chosen are the carcinoma colon samples that are predicted most active by the FOXO model of WO 2015/101635 A1: GSM523331, GSM523303, GSM523344 and GSM523323. Lastly, samples that have an inactive FOXO pathway are needed. These samples are chosen to be the most inactive carcinomas as predicted by the FOXO model of WO 2015/101635 A1: GSM523372, GSM523313, GSM523332 and GSM523283.

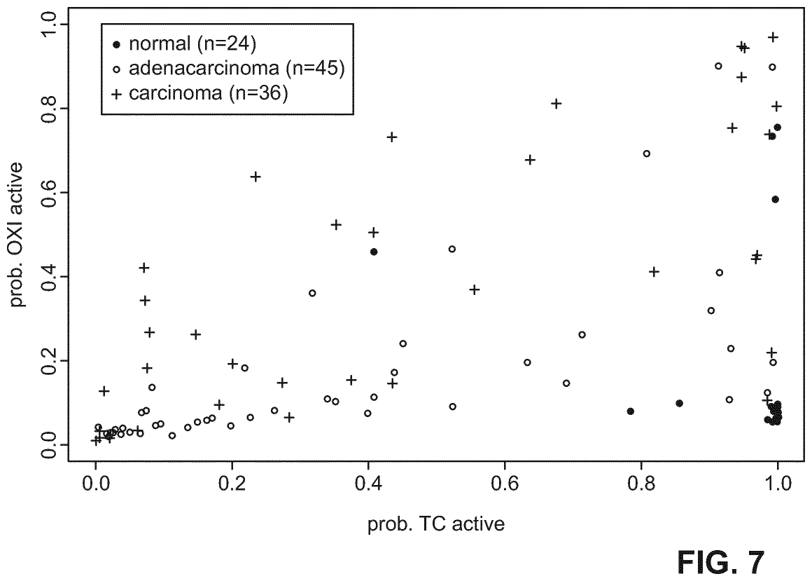

In FIG. 7, the described model was tested on the dataset GSE20916. For each sample in this set, the probability of the FOXO pathway to be active was calculated, the probability of the TC node, and the probability of the FOXO pathway to be in the oxidative stress state, i.e. the probability of the OXI node. Almost all normal colon samples, shown as black dots, are predicted to have an active FOXO pathway without oxidative stress, which is in line with the expectations. The adenocarcinoma and especially the colon carcinoma samples show a noticeably different combination of the FOXO and oxidative stress-induced activity compared to the normal samples. This shows that it is possible to distinguish normal FOXO activity from oxidative stress activity.

These results demonstrate the aforementioned FOXO target genes, PCK1, MXI1, SOD2 and BNIP3, to be indicative of oxidative stress-induced activity, in other words tumor-suppressive or—promoting activity of the FOXO pathway, and can be construed in such a Bayesian network that is able to better detect the tumor-suppressive or oxidative stress associated activity of the FOXO pathway, which may be tumor promoting. This is a novel and inventive technical enablement as the addition of the OXI node dependent on the nodes of a subset of FOXO target genes, indicative of the oxidative stress-induced activity of the FOXO pathway, as well as the node of the ‘normal’ FOXO activity results was not mentioned or hinted upon in the prior art.

The method presented herein may be used, for instance, in diagnosing an (abnormal) activity of the PI3K cellular signaling pathway, in prognosis based on the inferred activity of the PI3K cellular signaling pathway, in the enrollment of a medical subject in a clinical trial based on the inferred activity of the PI3K cellular signaling pathway, in the selection of subsequent test(s) to be performed, in the selection of companion diagnostics tests, in clinical decision support systems, or the like. In this regard, reference is made to the published international patent application WO 2013/011479 A2 (“Assessment of cellular signaling pathway activity using probabilistic modeling of target gene expression”) and to the published international patent application WO 2014/102668 A2 (“Assessment of cellular signaling pathway activity using linear combination(s) of target gene expressions”), which describe these applications in more detail.

### Example 3: Selection of the Genes Indicative of Oxidative Stress

For different tissue origins, differential mRNA expression of the oxidative stress-associated genes between cancer tissue with an active FOXO3 score as assessed by the FOXO3 model and corresponding normal tissue with an active FOXO3 score according to the model was determined. Gene expression levels as measured by gene-specific probesets on an Affymetrix microarray (GeneChip® Human Genome U133 Plus 2.0 Array) were obtained from public GEO datasets and averaged over a larger number of tissue samples from such a dataset. Subsequently, average mRNA expression levels for the oxidative stress associated genes BNIP3, MXI1, PCK1, PPARGGC1, SOD2, from non-malignant normal tissue were subtracted from the respective average level of the same mRNA measured in a cancer tissue sample, or a non-cancer condition, from the same tissue origin. E.g. normal lung average expression level for SOD2 gene (two probe sets, 215223_s and 216841_s) was subtracted from the average expression level of several different lung cancer subtypes. Subtracted tissue sample types are indicated to the left in table 4, the subtracted average mRNA expression level is indicated below each gene symbol to the right, for each probeset of the respective genes. The numbers in boxes indicate the level of differential expression and a positive value indicates that the subtraction resulted in a positive result; a negative level indicates a negative result. In the non-malignant conditions colon adenoma (A), Barrett's esophagus (C) and endometroid endometrium (E), oxidative stress-associated genes were not overexpressed. In contrast, in most cancer types (A-D, F-H), the oxidative stress-associated genes are overexpressed compared to corresponding normal tissue. Similar subtraction results are shown for FOXO3 and ESR expression levels, to indicate that these transcription factor levels were not differentially expressed. The GEO dataset containing samples from normal colon, colon adenoma and colon carcinoma (A) was used at the lead set to discover the oxidative stress-associated genes. The other GEO datasets (B-H) are presented to validate the oxidative stress gene panel in non-colon related cancer types and non-malignant conditions.

### Example 4: Use of SOD2 to Distinguish Between the Two Functional States of FOXO Activity

The PI3K signal transduction pathway is commonly hyper-activated in cancer. Tumors are potentially sensitive to PI3K pathway inhibitors but reliable diagnostic tests assessing functional PI3K activity are lacking. As the PI3K pathway negatively regulates FOXO transcription factors, FOXO target gene expression is inversely correlated to PI3K activity. A knowledge-based Bayesian computational model was developed inferring PI3K activity in cancer tissue samples using FOXO target gene mRNA levels. In various cancer cell lines it was observed with this model that addition of PI3K inhibitors caused an increase in FOXO activity, confirming a decrease in PI3K pathway activity. In tissue samples FOXO activity was predicted to be active in multiple cancer types of varying aggressiveness. Cellular oxidative stress is associated with cancer and an alternative activator of FOXO, frequently associated with PI3K pathway activity. SOD2 was found differentially expressed between the two modes of FOXO activation. Threshold levels for healthy tissue SOD2 expression were defined, above which FOXO activity was considered oxidative stress-induced. In slowly growing Luminal A breast cancer and low Gleason prostate cancer FOXO was typically active in PI3K-mediated fashion, indicating an inactive PI3K pathway. In more aggressive Luminal B, HER2 and basal-like breast cancer FOXO was frequently found either inactive, or active induced by oxidative stress, indicating high likelihood of PI3K pathway activity. A decision tree facilitates assessment of PI3K pathway activity in cancer samples. This mRNA-based FOXO-model may be used for response prediction of ErbB-PI3K pathway targeting drugs.

The past decade, systemic treatment of cancer moved from conventional chemotherapy towards administration of drugs that target tumor traits chosen on an individual patient basis. This “precision medicine” approach requires biomarkers that reliably predict response to targeted drugs (1). Cancer growth and metastasis are driven by roughly ten to twelve cellular signal transduction pathways, relatively independent of the cancer cell type of origin (2-4). One of these, the PI3K pathway, one of the main cellular growth factor signaling pathways, is frequently hyperactivated in cancer as a result of genomic changes like receptor tyrosine kinase amplification, loss of PTEN, activating mutations in PIK3CA, or by stimuli from the cancer cell micro-environment (2). PI3K pathway inhibitors are used in cancer treatment either alone or in combinations with other targeted strategies or conventional chemotherapy (5-7). Despite selection of potentially responsive patients based on PI3K pathway mutation analysis, only a subpopulation of patients respond adequately to the drugs (8,9). To improve prediction of drug response and monitor therapy efficacy or emerging resistance, tests measuring functional PI3K activity are needed.

Previously, a knowledge-based computational approach was described to assess signal transduction pathway activity in cancer tissue samples, based on measuring target gene mRNA levels of the pathway-specific transcription factor (10,11). Now development of an mRNA-based diagnostics for quantitative assessment of PI3K pathway activity is reported that uses Forkhead Box 0 (FOXO) transcription factor-induced transcription as readout, based on the well-known inverse relationship between FOXO and PI3K pathway activity (12-16). The model was biologically validated using breast and lung cancer cell lines treated with PI3K pathway inhibitors or carrying doxycycline inducible active FOXO3 constructs.

In cancer tissue FOXOs can be alternatively activated by cellular oxidative stress, a common trait in cancer which interferes with the inverse relationship with PI3K pathway activity. The SOD2/MnSOD FOXO target gene level was used to distinguish between the two functional states of FOXO activity, resulting in a robust approach to assess PI3K pathway activity in individual patient cancer samples.

### Methods

### Cell Culture-Based Model System for FOXO3 Activity

MCF7 and MDA-MB-231 breast cancer cell lines were cultured in DMEM-F12 containing 10% FBS (Lonza) 100 U/ml penicillin and 100 microgram/ml streptomycin (Lonza). Transfecting third generation packaging vectors using Polyethylenimine into HEK293T cells generated lentiviral particles (17). MCF7 and MDA-MB-231 cells were stably transduced with lentivirus containing pINDUCER20-FOXO3.A3, allowing doxycycline induced expression of constitutively active FOXO3 (FOXO3.A3) (13,18,19). Cells were treated with 20% FBS or 10 M PI3K inhibitor LY294002 (Selleckchem) for 16 hours to activate and inactivate the endogenous PI3K pathway, respectively. FOXO3.A3 expression was induced by 16 hours treatment with 10 ng/ml doxycycline.

### RNA Isolation and Affymetrix Microarray Hybridization

Treated cells were harvested after 16 hours of the respective incubations as indicated, RNA was isolated using the RNeasy kit (Qiagen), and hybridized on Affymetrix HT HG-U133+PM Array Plate by ServiceXS (GenomeScan, Leiden, The Netherlands, http://www.genomescan.nl) and Eurofins AROS Denmark (http://arosab.com/).

### Quality Control on Affymetrix

On all Affymetrix microarray data, both from experiments performed for this study, as well as from datasets from the public GEO database, an extensive quality control was performed. All microarrays that were used were from either Affymetrix HG-U133Plus2.0 or Affymetrix HG-U133+PM microarrays, which have been processed with fRMA with ‘random effect’ summarization (23). In principle, the Affymetrix HG-U133+PM platform contains all the perfect match probes of the HG-U133Plus2.0 platform, albeit with some minor reselections. To make the processed data comparable from both microarray types, a chip description file was used that contains only the shared probes, and took the processing parameters of this subset from the HG-U133Plus2.0 frmavecs to process data from both platforms.

Quality control of the microarray samples has been performed using several quality checks. These checks include the average value of all PM probes intensities, negative or extreme (>16-bit) intensity values, poly-A RNA (sample preparation spike-ins) and labelled cRNA (hybridization spike ins) controls, ACTB and GAPDH 3′/5′ ratio, values and center of intensity of the positive and negative border controls determined by the affyQCReport package and an RNA degradation value determined by the AffyRNAdeg function from the affy package. Samples from the breast and colon cancer datasets not passing the quality criteria were removed from further analyses.

Table 5 shows the datasets that have been used and in which Figures they appear

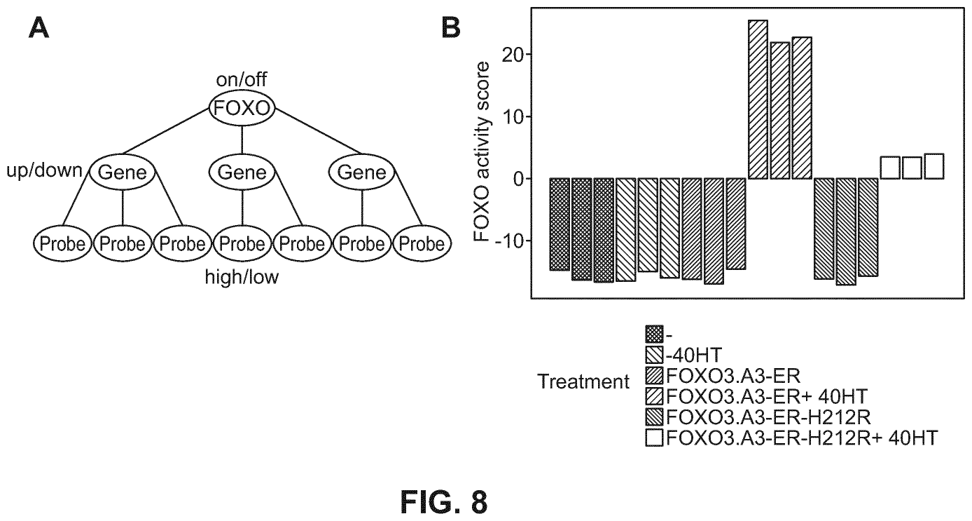

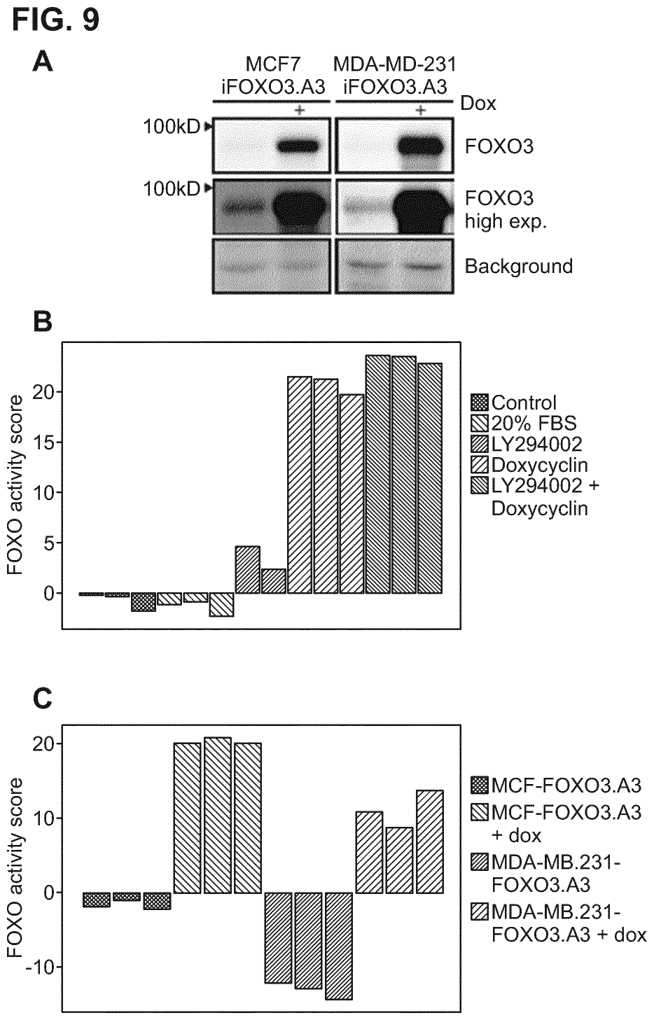

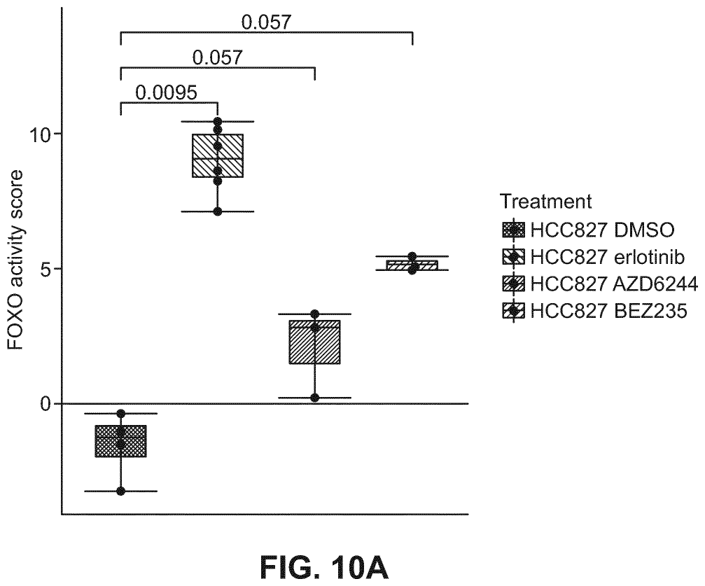

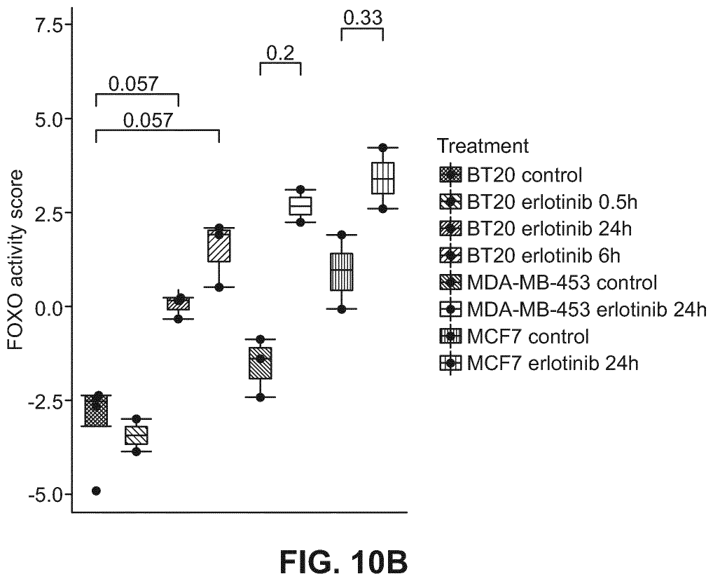

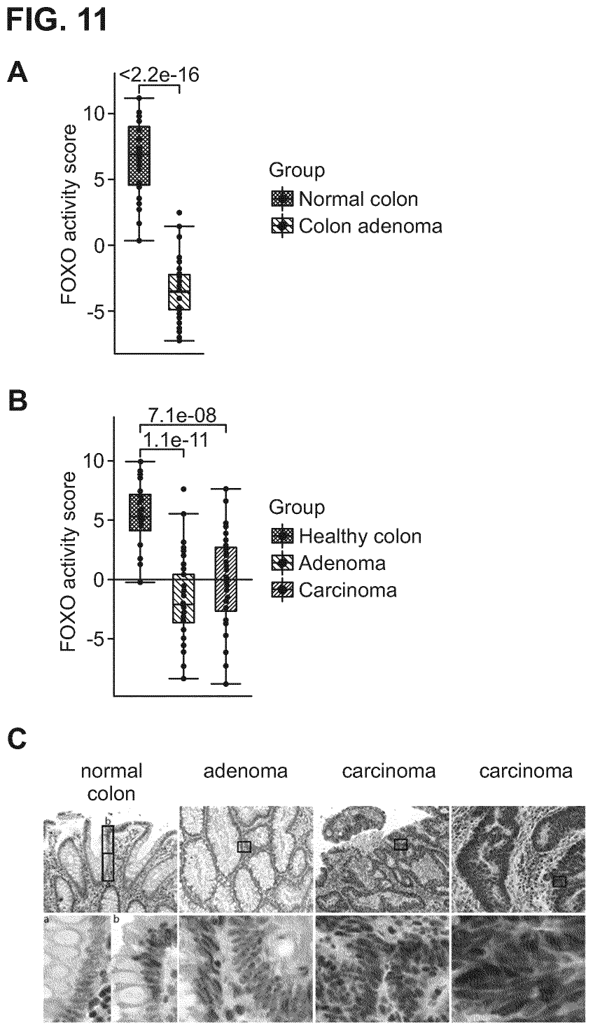

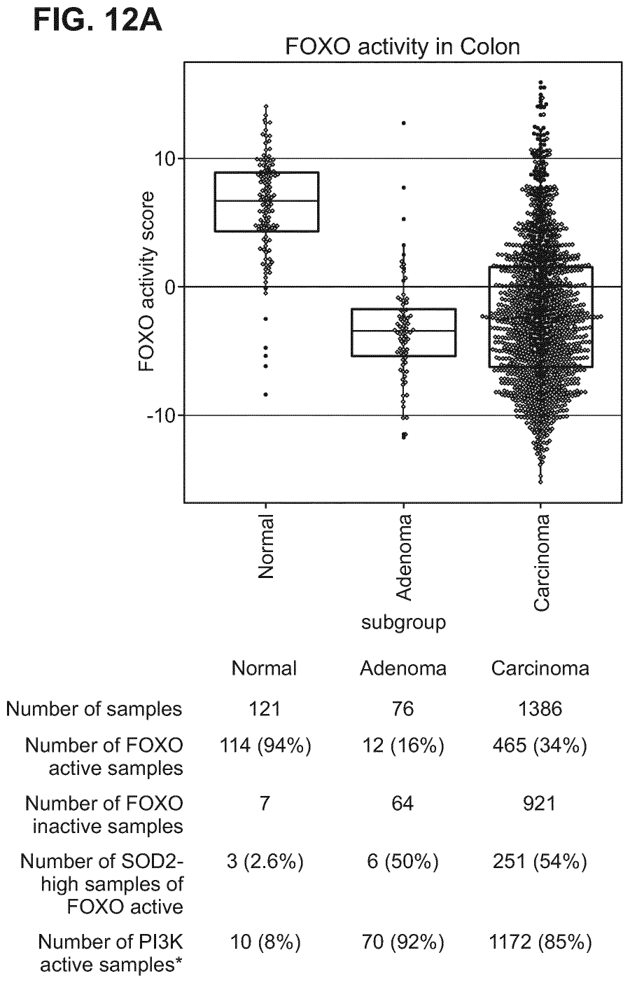

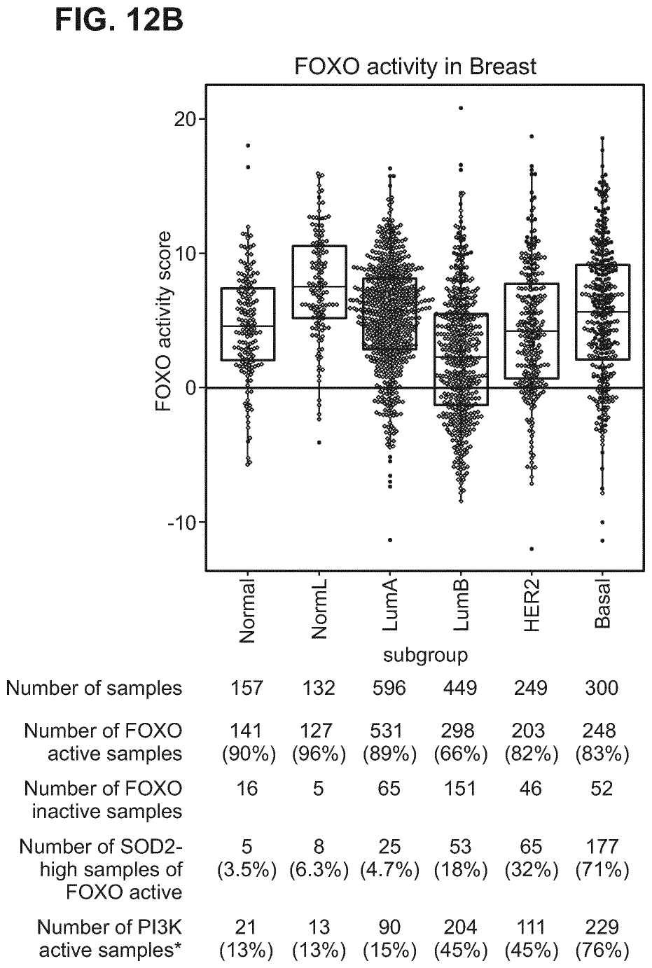

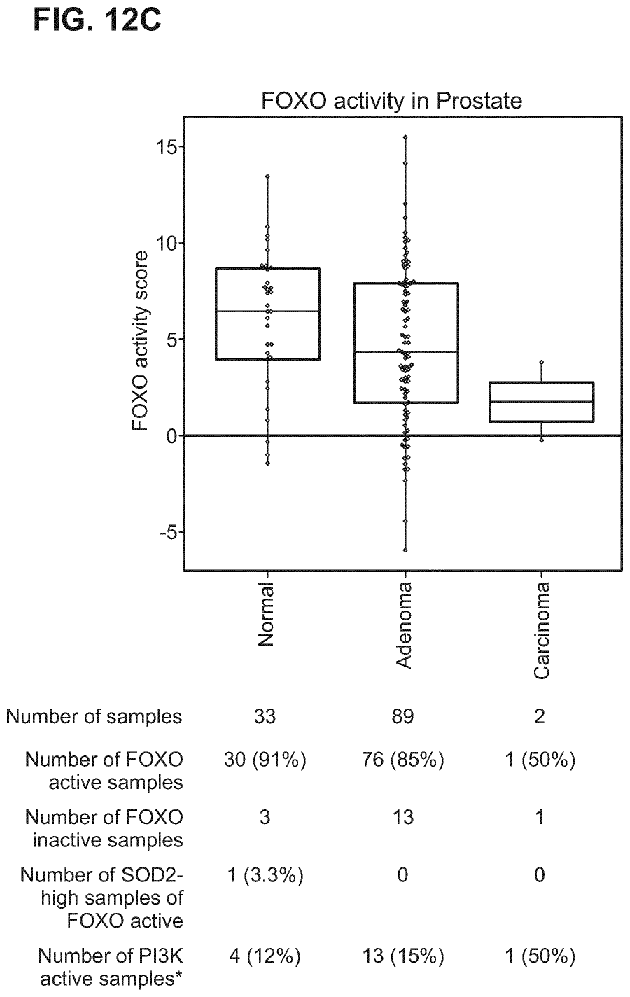

TABLE 5

| Dataset | Preprocessing | Purpose | Appearance |
|---|---|---|---|
| GSE16573 | fRMA | Calibration | FIG. 8 |
| Proprietary | PMfRMA | Validation | FIG. 9 |
| data | | | |
| GSE51212 | fRMA | Validation | FIG. 10 |
| GSE30516 | fRMA | Validation | FIG. 10 |
| GSE8671 | fRMA | Colon | FIG. 11 |
| GSE20916 | fRMA | Colon | FIG. 11 |
| GSE14333 | fRMA | Colon | FIG. 12 |
| GSE37364 | fRMA | Colon | FIG. 12 |
| GSE39084 | fRMA | Colon | FIG. 12 |
| GSE40967 | fRMA | Colon | FIG. 12 |
| GSE4183 | fRMA | Colon | FIG. 12 |
| E-MTAB-365 | fRMA | Breast | FIG. 12 |
| GSE10780 | fRMA | Breast | FIG. 12 |
| GSE12276 | fRMA | Breast | FIG. 12 |
| GSE17907 | fRMA | Breast | FIG. 12 |
| GSE20685 | fRMA | Breast | FIG. 5 |
| GSE21653 | fRMA | Breast | FIG. 5 |
| GSE26910 | fRMA | Breast | FIG. 5 |
| GSE42568 | fRMA | Breast | FIG. 5 |
| GSE45827 | fRMA | Breast | FIG. 5 |
| GSE6532 | fRMA | Breast | FIG. 5 |
| GSE7307 | fRMA | Breast | FIG. 5 |
| GSE9195 | fRMA | Breast | FIG. 5 |

### Western Blotting

Western blot analysis was performed using standard 6-15% SDS-PAGE. Proteins were detected with primary rabbit antibodies (1:2000) for FOXO3 (H144, Santa Cruz). Blots were incubated with HRP-conjugated secondary antibodies at 4° C. for 16 hours. Proteins were visualized with enhanced chemiluminescent (Biorad) using an ImageQuant LAS 4000 scanner (GE healthcare).

### Immunofluorescence and Immunohistochemistry

For immunofluorescent staining, cells were grown on glass coverslips, fixed using 4% paraformaldehyde and blocked with PBS containing 2% bovine serum albumin (BSA) (Invitrogen) and 0.1% normal goat serum (Invitrogen). Cells were incubated with FOXO3 antibody (Foxo3A Rabbit MAb, 1:500 CST-75D8), secondary Alexa563 conjugated antibodies and DAPI (Sigma). Slides were imaged on a Zeiss LSM710 confocal microscope.

For FOXO3 immunohistochemistry staining 4 μm sections of formalin fixed paraffin embedded (FFPE) tissue samples were deparaffinized and rehydrated. After blocking of endogenous peroxidase activity, antigen retrieval was performed with TE buffer at pH 9.0 (Dako) in a water bath at 95-96° C. for 25 minutes. After cooling for at least 15 minutes and washing steps with PBS the samples were blocked with 1% BSA in PBS for 15 minutes. Then the sections were incubated with FOXO3 antibody (1:50, CST-75D8) for 1 hour at room temperature. Visualization was achieved using the Dako Envision+TM—System anti-Rabbit-HRP (DAB). As a counterstain Gill's 2 Heamatoxilin is used. Images were generated with a 3D Histech scanner. Negative controls consisted of sections that underwent similar staining procedures without addition of the primary antibody. As a positive control non-malignant tonsil tissue is used.

### Development of the Computational Model for Predicting FOXO Activity

Development of the computational model for FOXO transcriptional activity is based on probabilistic Bayesian network inference, as previously described (11).

The signal transduction pathway modeling approach is based on inferring pathway activity from the expression profile of its target genes using probabilistic Bayesian network inference. Previously, such models were developed to determine the functional activity of the Wnt and ER pathways. Bayesian networks were built using the Bayes Net Toolbox for MATLAB, as described earlier. The Bayesian network structure used as a basis for the modeling approach (FIG. 8A) is a simplified model of the transcriptional program of a cellular signal transduction pathway, consisting of three types of nodes: (a) transcription complex, (b) target genes and (c) microarray probesets corresponding to target genes. The model describes (i) how expression of target genes depends on transcription complex activation, and (ii) how probeset intensities in turn depend on expression of the respective target genes.

The probabilistic relations in the Bayesian network model were made quantitative using experimental data to enable quantitative reasoning on new experimental samples. Parameters describing relationships between target genes and their respective probesets (ii) were trained on HUVEC cell lines with a stable transfection of a FOXO3.A3.ER construct stimulated for 12 hours with 4-OHT resulting in an active FOXO transcriptional program, used as FOXO3 active training samples, and without stimulation, used as FOXO3 inactive training samples (public data available in GSE16573 (20). With the PI3K pathway, the activity score of the pathway being active scales inversely with the probability of the FOXO3 transcription factor being in the active transcribing state. Parameters enforcing the relationships between the transcription complex and target genes (i) were manually set as described elsewhere (11) to improve generalization behavior of the model across different tissue types.

Once the model has been calibrated, it could be used on microarray (Affymetrix HG-U133Plus2.0) data of new tumor samples by entering probeset measurements as observations in the bottom layer, and inferring backwards in the model the activity probability of the FOXO3 transcription factor. The model was frozen and applied to microarray data of cell line and tissue samples by entering probeset measurements as observations into the model, inferring backwards FOXO transcription factor activity score as the log-2 value of the FOXO transcription factor odds p/(1−p). Samples are classified as FOXO active if the pathway activity exceeds an activity score above 0, corresponding with an odds above 1 to 1 that the pathway is active, and inactive in case activity score is below 0.

Parameters enforcing relationships between the FOXO transcription complex and target genes were manually set to improve generalization behavior of the model across different tissue types (11). Parameters describing relationships between target genes and their respective probe sets were calibrated on a public data set of Human Umbilical Vein Endothelial Cells (HUVEC) carrying inducible constitutively active FOXO3.A3-ER, and a threshold for FOXO-activity was set (GSE16573) (20). The model was frozen and applied to microarray data of cell line and tissue samples by entering probeset measurements as observations into the model, inferring backwards FOXO transcription factor activity score as the log-2 value of the FOXO transcription factor odds (p/(1-p). For validation purposes, FOXO activity analysis was always performed on independent fRMA (unless otherwise indicated) preprocessed Affymetrix HG-U133Plus2.0 microarray data from described experiments and from public GEO datasets.

### Identification of Direct Target Genes for FOXO3A

For optimal performance, across multiple different tissue types, the mathematical model should contain direct target genes of the FOXO transcription factor. Unfortunately, pathway databases such as KEGG (www.genome.jp/kegg) and Biocarta (www.biocarta.com) are incomplete and inconsistent on this aspect (23). Hence, target genes were manually selected based on extensive scientific evidence for each individual gene being a direct target gene of the respective transcription complex, including promotor region enhancer motif analysis, transcription factor binding experiments (EMSA and ChIP), gene promoter luciferase reporter experiments, and differential mRNA expression analysis. Available literature retrieved from the MEDLINE database using PubMed for FOXO target genes was extensively evaluated. In addition, target genes were extracted from Thomson-Reuters' Metacore by selecting only genes that had multiple sources of reliable evidence for being transcriptionally regulated by one or more of the FOXO family members. Ultimately, the target genes were ranked according to literature evidence using a similar methodology as described earlier (11). Only the highest ranked target genes, also included in the list published by van der Vos and Coffer (24) were selected as “bona fide” target genes.

SOD2 Level Associated with Oxidative Stress

To investigate differences in individual FOXO target gene expression levels between FOXO-active samples of healthy and tumor tissue, publicly available microarray datasets were used from healthy and corresponding pre-malignant or malignant tumor tissue samples (Table 7).

Table 7 shows the average expression levels (standard deviation in italics) of the two FOXO3 target genes SOD2 and BNIP3 as measured by the indicated probesets on the Affymetrix microarray, in samples scored as FOXO active by the computational FOXO model. GEO datset numbers are indicated in the table. A. Samples are selected from GEO samplesets based on a FOXO3 activity probability score (according to the FOXO model) above 5.6 (probability above 0.98); B. Samples are selected from GEO samplesets based on a FOXO3 activity probability score (according to the FOXO model) above 0.

Normalized intensities on a log 2-scale measured on the different gene-specific probesets on the microarray reflect gene expression levels. FOXO target gene expression levels were compared between FOXO-active tumor and healthy tissue samples.

To establish variation in SOD2 expression levels in the absence of oxidative stress, mean+2SD was determined for Affymetrix SOD2 probeset values for different types of healthy normal tissue. In FOXO-active samples with SOD2 mRNA levels exceeding these threshold levels, FOXO was considered as oxidative stress-induced.

Subtyping Breast Cancer Samples from Public Datasets According to Perou

Intrinsic breast cancer subtypes of all breast cancer samples were determined from microarray data according to the method described by Parker and co-workers (21,22). Intrinsic subtypes were determined from the Affymetrix microarray data using the methodology as described by Parker and co-workers (21) and the Prosigna Packet Insert (technologies, nanoString. Package Insert Prosigna Breast Cancer Prognostic Gene Signature Assay. s.l.: http://prosigna.com/docs/Prosigna_Packet_Insert_US.pdf, 2015). fRMA-normalized gene expression of all 50 genes included in PAM50 was extracted from the microarray data using the probesets associated with the PAM50-genes. The probeset with the highest variance was selected in case more than one probeset was associated with a single gene. Centroids for the luminal A, luminal B, HER2 enriched, basal and normal-likes were calculated using the samples from GSE21653 with given subtypes. Next, Pearson's correlation coefficients with these centroids were calculated for all samples. Each sample was assigned to the subtype with the highest correlation.

### Results

### Development of a Computational Model for PI3K-FOXO Activity

A Bayesian network-based computational model for FOXO activity was created which infers FOXO transcriptional activity from 26 FOXO target gene mRNA levels in a tissue sample (FIG. 8A). While inferring transcription factor activity from the mRNA level of one target gene is not sufficiently specific, inferring activity from expression levels of a larger number of target genes, typically between 20 and 30, appears to be a highly specific way to quantify associated transcription factor activity. For optimal performance across multiple different tissue types, direct target genes are selected. Since pathway databases such as KEGG (www.genome.jp/kegg) and Biocarta (www.biocarta.com) are inconsistent on this aspect (23), genes were manually selected based on scientific evidence from literature (PubMed) and Thomson-Reuters' Metacore, and ranked using a similar methodology as described earlier (11). Highest ranked genes were selected to build the computational FOXO pathway model (16,24) (Table 6). HUVECs with inducible FOXO activity provided “ground truth” evidence for FOXO activity status in an untransformed setting, and were used to calibrate the model, prior to model freeze (FIG. 8B).

Predictions of the Bayesian-model are consistent with known experimental FOXO activity status in the complete HUVEC data set including independent sample data (FIG. 8B). HUVECs, HUVECs treated with 4OHT, and HUVEC-FOXO3.A3-ER were predicted to have low FOXO activity and therefore active PI3K signaling, as expected in proliferating cells. HUVEC-FOXO3.A3-ER treated with 4OHT are predicted to have highly active FOXO, in line with the induction of constitutively active FOXO3.A3. In HUVECs expressing FOXO3.A3-ER-H212R, a mutant version of FOXO3 with reduced DNA binding capacity, the model predicted FOXO to be inactive in untreated cells and low FOXO3 activity in cells treated with 4OHT (20). These observations confirm that the model specifically detects transcriptional changes induced by FOXOs, and is sensitive to low levels of FOXO activity.

### Biological Validation of the PI3K-FOXO Model in Breast Cancer Cell Lines

Following calibration, the model was biologically validated in independent breast cancer cell lines. ER positive, PIK3CA^{E545K} mutant MCF7 and triple negative MDA-MB-231 cells were stably transduced with a doxycycline inducible FOXO3.A3 expression vector, allowing rapid and controlled induction of FOXO3 protein expression and transcriptional activity upon treatment with doxycycline for 16 hours (FIG. 9A). FOXO3 protein was predominantly detected in the cytoplasm in untreated and 20% FBS stimulated cells; switching to dominant nuclear localization in cells treated with doxycycline, PI3K inhibitor LY294002, and doxycycline in combination with LY294002. This shows that nuclear translocation of FOXO3 was induced in a controlled manner in this experimental cell culture system. On Affymetrix mRNA expression data from this cell model, the FOXO model predicted respectively low FOXO activity in untreated (PI3K pathway active), and high FOXO activity in doxycycline-treated (PI3K pathway inactive) MCF-FOXO3.A3 and MDA-MB-231-FOXO3.A3 cells; low FOXO activity in 20% FBS treated MCF7 cells (PI3K pathway active), and high activity in doxycycline, LY294002 and combined doxycycline+LY294002 treated cells (all PI3K pathway inactive) (FIG. 9B/C). Together these results confirm that the computational FOXO model predicted FOXO activity as expected in independent cancer cell line samples.

### Use of the Pathway Model to Predict and Monitor Response to Drugs.

On the premise that FOXO activity is inversely related to PI3K activity in cell lines, it was investigated whether the FOXO model was capable of predicting response to drugs targeting receptor tyrosine kinase activity in independent cancer cell line data sets (GSE51212, GSE30516). FOXO3 activity was scored in an EGFR-mutant HCC827 lung cancer cell line either treated with Erlotinib (EGFR inhibitor), Selumetinib (MEK inhibitor) or BEZ235 (PI3K/TOR dual inhibitor). FOXO scored inactive in untreated samples indicating the active PI3K pathway; upon treatment with any of the three drugs FOXO scored active, maximal with the EGFR inhibitor, confirming that all three drugs were effective in directly and/or indirectly blocking activity of the PI3K pathway (FIG. 10A). In three breast cancer cell lines representing triple-negative (BT20), ER positive (MCF7), and HER2 positive breast cancer (MDA-MB-453) the FOXO activity score increased as expected when treated with Erlotinib (FIG. 10B).

### FOXO Activity in Healthy Colon and Colorectal Cancer Tissue Samples

For evaluation of the model when used on patient tissue samples, a number of selected public datasets were used. First the FOXO activity model was applied on tissue samples derived from patient biopsies of 32 normal colon tissues and 32 adenoma tissues (GSE8671). A clear difference in FOXO activity score was observed between healthy colon and adenoma tissue, showing FOXOs to be active and inactive respectively, indicating expected activation of the PI3K pathway in colon adenoma (FIG. 11A).

Second, the FOXO model was applied on a patient tissue set containing normal colon, benign colon adenoma and colon carcinoma tissues (GSE20916) (FIG. 11B). In normal colon tissue samples FOXO was predicted active. In the majority of adenoma tissue samples FOXO was predicted to be inactive, in line with the findings in the first data set, and indicating PI3K pathway activity. However, in half of the colon carcinoma tissue samples FOXOs were predicted to be active. Since colon carcinomas are thought to arise from colon adenomas, at least the same frequency of PI3K pathway activity was expected.

Thus, in up to about one third of the cancer tissue samples a high FOXO activity score would be expected to indicate an inactive PI3K pathway. In the rest of the FOXO active cancer samples there may be another cause of high FOXO activity, one being admixture of healthy cells in which FOXO is generally active. To investigate this, a FOXO3 immunohistochemistry (IHC) staining was developed to determine cytoplasmic and nuclear FOXO3 localization in tissue samples. FOXO3 was found mainly cytoplasmic in healthy colon crypt cells but present in the nucleus in healthy mucosa cells, and in other non-tumor healthy cells, in line with the mRNA-based FOXO-activity score seen in the public dataset GSE20916 (but not the same samples) (FIG. 11B/C). These results suggest that admixture of normal cells may cause a false positive FOXO score. In colon adenoma cells FOXO3 showed cytoplasmic localization while colon carcinoma cells explicitly displayed heterogeneous FOXO3 localization with areas of nuclear and other areas with cytoplasmic staining.

Another well-described cause for FOXO activity likely to be present in aggressive cancer tissue is cellular oxidative stress (12,25). However, the function of FOXO activity in normal tissue is clearly different from the function during oxidative stress, and is therefore likely to be reflected in a difference in transcribed target genes.

To determine which genes within the used FOXO target gene set would discriminate best between FOXO activity in normal and in carcinoma samples, target gene expression levels were compared between normal colon and colon cancer tissue samples in which FOXOs were predicted to be active by the model. SOD2 and BNIP3 gene expression was strongly increased in FOXO-active cancer tissue samples compared to normal colon tissue (Table 7). Both genes play a role in response to oxidative stress and are transcribed by FOXOs under these circumstances, making them prime candidates to distinguish between the two modes of FOXO activity (26,27).

Indeed, comparing SOD2 and BNIP gene expression levels between various FOXO active normal tissues and corresponding pre-malignant or malignant tumor tissue samples (colon cancer, breast cancer, Barrett's esophagus, esophageal cancer, bladder cancer, and gliomas) confirmed increased expression levels of SOD2 and to a lesser extent BNIP3 in FOXO-active samples from aggressive cancer types (Table 7). In remarkable contrast, in FOXO active samples of two benign hyperproliferative conditions, colon adenoma and Barrett's esophagus, FOXO activity was not associated with increased SOD2 and BNIP3 expression, indicating that in these benign tumors FOXO was activated in a PI3K-regulated manner comparable to healthy tissue. Since SOD2 showed the most generalized and profound differential expression between FOXO-active normal tissue samples and corresponding FOXO-active cancer types, this gene was selected as the most reliable parameter to distinguish between the two modes of FOXO activity. The SOD2 upper threshold level for PI3K-regulated (non-oxidative stress) FOXO activity was defined as two standard deviations above the mean expression level in normal tissue, and was calculated for healthy colon, breast and prostate tissue.

Subsequently, the FOXO model in combination with the SOD2 expression threshold was applied to independent publicly available data sets with data from individual patients with colon, breast, and prostate cancer. In FOXO-active samples with elevated SOD2 expression PI3K pathway activity cannot be directly inferred from FOXO activity. Also, in case FOXO is scored inactive by the model, knowledge on FOXO expression is required for a conclusion with regard to PI3K activity. FOXO3 is considered the most relevant FOXO gene in cancer and was consistently expressed in all samples from all cancer types that were analysed to date, including from breast, colon, prostate, brain, bladder, and esophagus (Table 7).

### Prediction of FOXO Activity Mode (Canonical or Oxidative Stress) in Primary Colon Adenoma and Carcinoma Tissue Samples

Analysis of a large set of healthy colon tissue samples (n=121) allowed a threshold setting for normal SOD2 mRNA levels in FOXO-active samples (FIG. 12A). Subsequently an extended independent set of patient colon adenoma and carcinoma sample data was compiled and FOXO activity and SOD2 expression levels determined. In normal colon samples only 2.6% of FOXO active samples had SOD2 expression exceeding the threshold level. In the few adenoma samples that were FOXO active (n=12, 16%), SOD2 levels were elevated (over threshold) in half. In the carcinoma samples one third of the samples were scored FOXO active, of which 53.9% with elevated SOD2 expression.

### Prediction of FOXO Activity Mode (Canonical Versus Oxidative Stress) in Primary Breast Cancer Tissue Samples

Similarly a compiled set of data from breast cancer patients was analyzed. Prior to FOXO activity analysis, breast cancer tumor subtyping in all cancer data sets was performed using the PAM50 algorithm to ensure that the subtypes in all data sets were similarly determined (21). In line with the findings in healthy colon tissue FOXO was predicted to be generally active in normal breast tissue (85%) (FIG. 12B). In the luminal B, HER2 and basal like subtypes respectively 37%, 23% and 20% scored low for FOXO, indicating PI3K pathway activity. An increasing percentage of FOXO active samples with elevated SOD2 (over threshold in healthy breast cancer) was observed with increasing cancer subtype aggressiveness: from 4.7% in luminal A to 71.4% in basal like breast cancer.

### Prediction of FOXO Activity Mode (Canonical Versus Oxidative Stress) in Primary Prostate Cancer Samples

In normal prostate tissue and primary prostate cancer samples from compiled public patient datasets FOXO activity analysis showed that FOXO is active in 91% of patients with lower Gleason score tumors (Gleason 4-7). Too few samples of patients with a high Gleason score (Gleason 8-9) were available to draw any comparative conclusions (FIG. 12C). Interestingly, in none of the FOXO-active primary prostate cancer samples SOD2 expression was increased over the threshold level set for normal tissue, indicating that for prostate cancer PI3K pathway activity can be safely inferred from the FOXO activity score. In the majority of primary prostate cancer samples the PI3K pathway was inactive.

Identifying Tumors with an Active PI3K Pathway

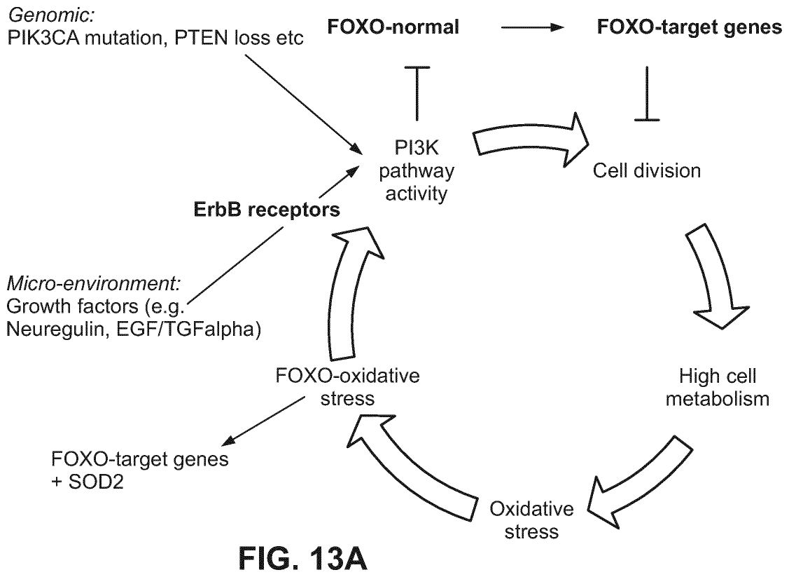

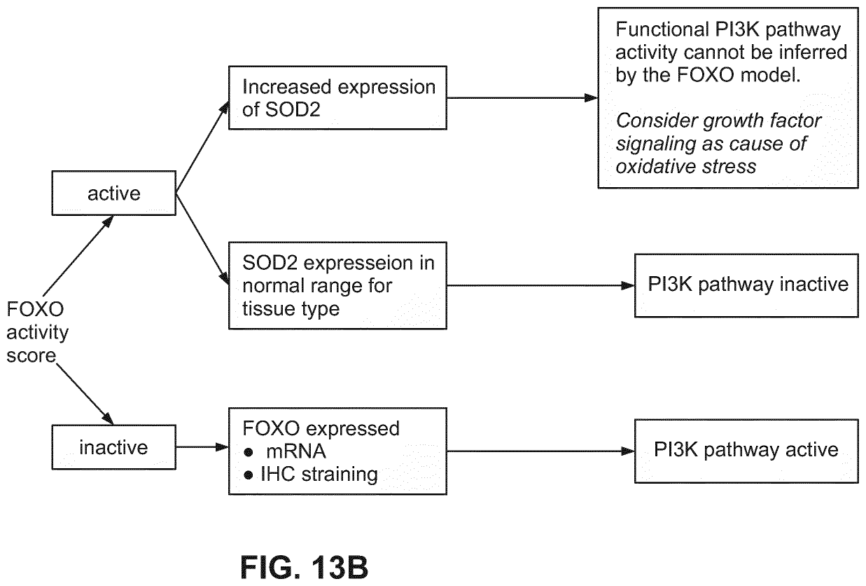

To facilitate inference of functional PI3K pathway activity in a tissue sample, a simplified decision tree was created, based on the premises (1) that FOXO is expressed in the cancer cells from the sample and (2) measured FOXO activity derives from cancer cells (FIG. 13B). Assuming the PI3K pathway is active in case of active FOXO-oxidative stress, for each investigated cancer type these samples can be added (FIG. 12, FOXO active black dots) to the number of samples with inactive FOXO (indicating active PI3K pathway) to calculate the total number/percentage of patients likely to have a tumor with an active PI3K pathway (FIG. 12, indicated in the table with asterix). The thus calculated percentage of PI3K pathway active samples was between 8 and 13% in normal tissue (colon, breast, prostate), and quite similar in Luminal A and Normal-like breast cancer and low Gleason score prostate cancer (respectively 13%, 15%, 15%); in Luminal B, HER2, and basal-like breast cancer the percentage samples with PI3K pathway activity was much higher, respectively 45%, 45%, and 76%; while in colon adenoma and carcinoma nearly all samples were scored as PI3K pathway active (respectively 92% and 85% of samples).

### Discussion

### A Knowledge-Based Bayesian Model for Predicting FOXO and PI3K Activity

The PI3K pathway is an important proliferation and survival pathway in cancer and is a core signaling part of the ErbB growth factor signal transduction machinery. Many targeted drugs aim at blocking PI3K pathway activity at multiple locations in the pathway. Improving response rates to PI3K pathway inhibitors requires tests that reliably assess PI3K pathway activity in cancer samples. FOXO transcription factors are negatively regulated by the PI3K pathway, and can in principle be used as an inverse readout for PI3K pathway activity (5,12,28). To measure PI3K pathway activity in cancer tissue samples, a computational knowledge-based Bayesian network was developed inferring FOXO transcriptional activity from established FOXO target gene mRNA expression levels to be measured in the tissue sample at hand (11). The different FOXO members are redundant and a comparative analysis of FOXO1, FOXO3 and FOXO4-induced gene regulation indicates a large overlap between the transcriptional profiles of each FOXO member (16,24,29). Therefore the FOXO activity model, which incorporates knowledge on regulation of direct FOXO target genes, performs as a general FOXO activity predictor.

Calibrating this Bayesian model on FOXO-inducible HUVECs resulted in a computational FOXO model that predicted FOXO activity as expected in breast cancer cell lines with either constitutively active FOXO3, or incubated with PI3K pathway targeting drugs. The observation in MCF7 cells that FOXO is scored less active in cells treated with the PI3K inhibitor drug LY294002 compared to ectopic FOXO activation is readily explained by higher FOXO3 protein levels induced by ectopic expression, and/or as a result of positive growth factor signaling feedback in the case of treatment with LY294002. Pharmacological inhibition of the PI3K pathway may initiate a growth factor feedback response that can re-establish growth factor signaling which constitutes a major component of drug resistance development in cancer cells (6,7,30). In contrast to MCF7 cells, in untreated MDA-MB-231 cells some nuclear FOXO protein was detected. However the FOXO activity model scored this FOXO as transcriptionally inactive—as expected in this rapidly dividing cell line. Thus the nuclear presence of FOXO cannot always be used to infer transcriptional activity.

In three breast cancer cell lines as well as in lung cancer cells with mutated hyperactive EGFR, the model identified increased FOXO activity, associated with effective inhibition of PI3K pathway activity by the EGFR inhibitor erlotinib. In addition, treatment of the lung cancer cell line with a dual mTOR-PI3K inhibitor (BEZ235), and a MEK1/2 inhibitor (selumetinib) similarly resulted in increased FOXO activity, and thus reduced inferred PI3K pathway activity. Signal transduction initiated by the ErbB family of growth factor receptors (EGFR, HER2, HER3, HER4) leads to inactivation of FOXO via PI3K-AKT and RAS-MEK-ERK-MDM2 (31).

Thus the Bayesian model robustly predicted FOXO and PI3K activity in these cell culture-based models for PI3K pathway activation, providing biological validation of the model. However, the difference in response of the lung cancer cell line to the three drugs targeting different elements of the signaling pathways downstream of EGFR illustrates that to make an optimal choice with respect to a targeted drug, additional analysis (e.g. genomic mutation analysis) may be required to establish the underlying cause of the PI3K pathway activity.

### A Computational Model Predicting FOXO Activity in Tissue Samples

Applying the model to tissue material to infer PI3K activity proved to be more challenging. In healthy tissue samples from a variety of tissue types (colon, breast, prostate, esophagus, bladder, brain) FOXO was scored active, as expected. In colon adenoma samples FOXO activity was frequently lost and PI3K pathway inferred as active, once more emphasizing the role of FOXO in controlling cell division. In this situation growth factor-induced activation of the PI3K pathway initiates mitogenic signaling and blocks FOXO transcriptional activity (12,32,33,34).

### Distinguishing Between Different Mechanisms Regulating FOXO Activity

In cancer the situation in which FOXOs can be active becomes more complex. Both in colon carcinoma and luminal B, HER2 and basal like breast carcinoma subtypes, where the PI3K pathway is frequently activated, the majority of the samples were predicted to have an active FOXO transcription factor (2,35). This suggested that simple inversion of FOXO activity status to infer PI3K pathway activity was not valid in all cancer tissue samples. A differentiator between canonical FOXO activity associated with control of cell division and FOXO activity in the presence of growth factor/PI3K activity-induced cell proliferation was necessary.

While considered tumor suppressors, FOXOs also function as regulators of cellular homeostasis and respond to various adverse cellular conditions including DNA damage, high levels of reactive oxygen species and low nutrient availability (12,36,37). Indeed, in cancer samples, an active PI3K pathway may be present in combination with active FOXO transcription since FOXO can be activated in an alternative manner via oxidative stress to protect cells against ROS (FIG. 13A). Such an oxidative stress state is a frequent phenomenon in hypoxic and rapidly proliferating cancer tissues (12,38-40).

FOXO is post-translational modified to adapt its transcriptional function to different intracellular conditions (41). It was hypothesized that the two very different functional roles of FOXO would be reflected in changes in FOXO target mRNA expression profiles. BNIP3 and SOD2 are two FOXO3 target genes used by the computational model and known to be induced in the presence of cellular oxidative stress to protect the cell against the consequences of this toxic state (26,27,42). Indeed, high expression of both SOD2 and BNIP3 was found by comparing FOXO target gene expression level between multiple FOXO-active cancer samples and corresponding healthy tissue samples, in contrast to FOXO-active healthy tissue and benign hyper-proliferative colon adenoma. These results underpin the concept that oxidative stress in parallel to growth factor signalling can by an alternative pathway induce FOXO activity in rapidly proliferating cancer tissue. Of the two genes, SOD2 expression appeared to be universally elevated in FOXO-active samples from a large number of cancer types when compared to healthy tissues. To enable distinction between canonical FOXO activity and oxidative stress-associated FOXO activity, threshold levels for SOD2 expression in healthy tissues were defined. Adding this information to the FOXO model improves reliability of inferring PI3K pathway activity in various types of cancer.

The application of this rule to breast and prostate cancer subtypes that are well-defined with respect to aggressive behaviour and prognosis allowed initial clinical validation. Indeed, adding information on SOD2 expression level to the FOXO activity score classified FOXO activity in luminal A breast cancer and lower Gleason score prostate cancer in general as canonical FOXO activity, with an inferred inactive PI3K pathway. Indeed, these cancer types are typically more differentiated and slow growing and driven by the ER pathway in the absence of an active HER2-PI3K pathway. In contrast, in breast cancer subtypes with more aggressive behaviour, that is luminal B, HER2 and basal like, samples where either inactive for FOXO (indicating an active PI3K pathway) or FOXO activity was increasingly classified as FOXO oxidative stress activity. Summarizing, in well differentiated slow growing breast and prostate cancer FOXOs were generally active in the canonical mode to control cell division, while in more aggressive cancer FOXOs were either inactivated by PI3K pathway activation, or active in the oxidative stress mode. Interestingly, oxidative stress-associated FOXO activity may actually function to support tumor growth by both protecting the cell from oxidative stress damage which normally leads to cell death, and stimulating the growth factor PI3K-AKT pathway (43).

### Use of the Computational FOXO Model to Identify PI3K Pathway Activity in an Individual Cancer Tissue Sample

The use of the computational model is to assess functional PI3K pathway activity in an individual cancer tissue samples to support in the decision on the choice of a targeted drug directed at ErbB growth factor signaling pathways, e.g. HER2-PI3K and EGFR pathways, and/or to monitor response and detect emerging resistance against the chosen drug. Using the FOXO computational model to assess FOXO activity in combination with the oxidative stress SOD2 gene marker which indicates the mode of FOXO activity, the likelihood that the PI3K pathway is active can be derived from highly specified gene expression data using a (somewhat simplified) decision tree (FIG. 13B). In case FOXO is scored active, expression levels of SOD2 are interpreted to decide on PI3K-regulated versus oxidative stress induced FOXO activity. If FOXO activity is caused by oxidative stress, underlying information on activity of the PI3K pathway is hidden, and formally a decision cannot be made with respect to activity of the PI3K pathway. However, cellular oxidative stress associated FOXO activity frequently (if not always) occurs concurrently with growth factor pathway activity in high-grade cancers (4,38). Thus, in the presence of FOXO active/SOD2 high, there is a high likelihood of the PI3K pathway being active. Adding up samples that were scored as either FOXO inactive or FOXO active-oxidative stress provided an indication of the total number of patients within each cancer subtype group with a PI3K active tumor which might have benefited from PI3K pathway inhibitor treatment. In the low grade Luminal A and lower Gleason score prostate cancer groups the thus calculated percentage of PI3K-active tumors was close to that seen in healthy tissue (roughly around 10%), while in higher grade breast cancer this increased up to three quarters in the basal-like breast cancer group. Indeed, the PI3K pathway is probably the most frequently activated pathway in cancer.

When in a tissue sample FOXOs are predicted to be active and expression levels of SOD2 fall within the normal range, complementary IHC staining may be necessary to exclude that FOXO activity is caused by healthy tissue cell contamination of the cancer sample. Taking this into account, the FOXO model provides a robust method for determining functional PI3K pathway activity in tumor samples. Targeted genomic analysis to identify a mutational cause of abnormal PI3K pathway activity can be limited to patients in which an active PI3K pathway was inferred. This approach is expected to improve decision taking on the use of drugs targeting the ErbB PI3K-AKT-mTOR pathway aiming at increased treatment efficacy rates, and is expected to be applicable to many different tumor types. Monitoring of therapy response and resistance, for example in a neo-adjuvant or “window-of-opportunity” setting, is another envisioned application, as is quantitative assessment of drug response during drug development.

### REFERENCES

1. Ashley E A. Towards precision medicine. Nat Rev Genet 2016; 17(9):507-22.
2. Vogelstein B, Papadopoulos N, Velculescu V E, Zhou S, Diaz L A, Jr., Kinzler K W. Cancer genome landscapes. Science 2013; 339(6127):1546-58.
3. van de Stolpe A. On the origin and destination of cancer stem cells: a conceptual evaluation. Am J Cancer Res 2013; 3(1):107-16.
4. Hanahan D, Weinberg R A. Hallmarks of cancer: the next generation. Cell 2011; 144(5):646-74.
5. Fruman D A, Rommel C. PI3K and cancer: lessons, challenges and opportunities. Nat

Rev Drug Discov 2014; 13(2):140-56.
6. Engelman J A. Targeting PI3K signalling in cancer: opportunities, challenges and limitations. Nat Rev Cancer 2009; 9(8):550-62.
7. Arnedos M, Vicier C, Loi S, Lefebvre C, Michiels S, Bonnefoi H, et al. Precision medicine for metastatic breast cancer-limitations and solutions. Nature reviews Clinical oncology 2015.
8. Rodon J, Dienstmann R, Serra V, Tabernero J. Development of PI3K inhibitors: lessons learned from early clinical trials. Nat Rev Clin Oncol 2013; 10(3):143-53.
9. Kwiatkowski D J, Wagle N. mTOR Inhibitors in Cancer: What Can We Learn from Exceptional Responses? EBioMedicine 2015; 2(1):2-4.
10. Verhaegh W, Van de Stolpe A. Knowledge-based computational models. Oncotarget 2014; 5(14):5196-7.
11. Verhaegh W, van Ooijen H, Inda M A, Hatzis P, Versteeg R, Smid M, et al. Selection of personalized patient therapy through the use of knowledge-based computational models that identify tumor-driving signal transduction pathways. Cancer Res 2014; 74(11):2936-45.
12. Eijkelenboom A, Burgering B M. FOXOs: signalling integrators for homeostasis maintenance. Nat Rev Mol Cell Biol 2013; 14(2):83-97.
13. Brunet A, Bonni A, Zigmond M J, Lin M Z, Juo P, Hu L S, et al. Akt promotes cell survival by phosphorylating and inhibiting a Forkhead transcription factor. Cell 1999; 96(6):857-68.
14. Kops G J, de Ruiter N D, De Vries-Smits A M, Powell D R, Bos J L, Burgering B M. Direct control of the Forkhead transcription factor AFX by protein kinase B. Nature 1999; 398(6728):630-4.
15. Eijkelenboom A, Mokry M, de Wit E, Smits L M, Polderman P E, van Triest M H, et al. Genome-wide analysis of FOXO3 mediated transcription regulation through RNA polymerase II profiling. Mol Syst Biol 2013; 9:638.
16. Webb A E, Kundaje A, Brunet A. Characterization of the direct targets of FOXO transcription factors throughout evolution. Aging Cell 2016; 15(4):673-85.
17. Dull T, Zufferey R, Kelly M, Mandel R J, Nguyen M, Trono D, et al. A third-generation lentivirus vector with a conditional packaging system. J Virol 1998; 72(11):8463-71.
18. Meerbrey K L, Hu G, Kessler J D, Roarty K, Li M Z, Fang J E, et al. The pINDUCER lentiviral toolkit for inducible RNA interference in vitro and in vivo. Proc Natl Acad Sci USA 2011; 108(9):3665-70.
19. Hornsveld M, Tenhagen M, van de Ven R A, Smits A M, van Triest M H, van Amersfoort M, et al. Restraining FOXO3-dependent transcriptional BMF activation underpins tumour growth and metastasis of E-cadherin-negative breast cancer. Cell Death Differ 2016.
20. Czymai T, Viemann D, Sticht C, Molema G, Goebeler M, Schmidt M. FOXO3 modulates endothelial gene expression and function by classical and alternative mechanisms. J Biol Chem 2010; 285(14):10163-78.
21. Parker J S, Mullins M, Cheang M C, Leung S, Voduc D, Vickery T, et al. Supervised risk predictor of breast cancer based on intrinsic subtypes. J Clin Oncol 2009; 27(8):1160-7.
22. Perou C M, Sorlie T, Eisen M B, van de Rijn M, Jeffrey S S, Rees C A, et al. Molecular portraits of human breast tumours. Nature 2000; 406(6797):747-52.
23. Shmelkov E, Tang Z, Aifantis I, Statnikov A. Assessing quality and completeness of human transcriptional regulatory pathways on a genome-wide scale. Biol Direct 2011; 6:15.
24. van der Vos K E, Coffer P J. The extending network of FOXO transcriptional target genes. Antioxid Redox Signal 2011; 14(4):579-92.
25. van den Berg M C W, Burgering B M T. Integrating opposing signals toward forkhead box o. Antioxidants &amp; redox signaling 2011; 14(4):607-21.
26. Kops GJPL, Dansen T B, Polderman P E, Saarloos I, Wirtz K W A, Coffer P J, et al. Forkhead transcription factor FOXO3a protects quiescent cells from oxidative stress. Nature 2002; 419(6904):316-21.
27. Mammucari C, Milan G, Romanello V, Masiero E, Rudolf R, Del Piccolo P, et al. FoxO3 controls autophagy in skeletal muscle in vivo. Cell Metab 2007; 6(6):458-71.
28. Kim H J, Lee S Y, Kim C Y, Kim Y H, Ju W, Kim S C. Subcellular localization of FOXO3a as a potential biomarker of response to combined treatment with inhibitors of PI3K and autophagy in PIK3C A-mutant cancer cells. Oncotarget 2017; 8(4):6608-22.
29. Paik J H, Kollipara R, Chu G, Ji H, Xiao Y, Ding Z, et al. FoxOs are lineage-restricted redundant tumor suppressors and regulate endothelial cell homeostasis. Cell 2007; 128(2):309-23.
30. Chandarlapaty S, Sawai A, Scaltriti M, Rodrik-Outmezguine V, Grbovic-Huezo 0, Serra V, et al. AKT inhibition relieves feedback suppression of receptor tyrosine kinase expression and activity. Cancer Cell 2011; 19(1):58-71.
31. Yang J Y, Zong C S, Xia W, Yamaguchi H, Ding Q, Xie X, et al. ERK promotes tumorigenesis by inhibiting FOXO3a via MDM2-mediated degradation. Nat Cell Biol 2008; 10(2):138-48.
32. Sheng H, Shao J, Townsend C M, Jr., Evers B M. Phosphatidylinositol 3-kinase mediates proliferative signals in intestinal epithelial cells. Gut 2003; 52(10):1472-8.
33. Clevers H. The intestinal crypt, a prototype stem cell compartment. Cell 2013; 154(2):274-84.
34. Clemons N J, Phillips W A, Lord R V. Signaling pathways in the molecular pathogenesis of adenocarcinomas of the esophagus and gastroesophageal junction. Cancer Biol Ther 2013; 14(9):782-95.
35. Vanhaesebroeck B, Stephens L, Hawkins P. PI3K signalling: the path to discovery and understanding. Nat Rev Mol Cell Biol 2012; 13(3):195-203.
36. van der Horst A, Burgering B M. Stressing the role of FoxO proteins in lifespan and disease. Nat Rev Mol Cell Biol 2007; 8(6):440-50.
37. Webb A E, Brunet A. FOXO transcription factors: key regulators of cellular quality control. Trends Biochem Sci 2014; 39(4):159-69.
38. Hornsveld M, Dansen T B. The Hallmarks of Cancer from a Redox Perspective.

Antioxid Redox Signal 2016; 25(6):300-25.
39. Klotz L O, Sanchez-Ramos C, Prieto-Arroyo I, Urbanek P, Steinbrenner H, Monsalve M. Redox regulation of FoxO transcription factors. Redox Biol 2015; 6:51-72.
40. van den Berg M C, van Gogh I J, Smits A M, van Triest M, Dansen T B, Visscher M, et al. The small GTPase RALA controls c-Jun N-terminal kinase-mediated FOXO activation by regulation of a JIP1 scaffold complex. J Biol Chem 2013; 288(30):21729-41.
41. Calnan D R, Brunet A. The FoxO code. Oncogene 2008; 27(16):2276-88.
42. Lin A, Yao J, Zhuang L, Wang D, Han J, Lam E W, et al. The FoxO-BNIP3 axis exerts a unique regulation of mTORC1 and cell survival under energy stress. Oncogene 2014; 33(24):3183-94.
43. Coomans de Brachene A, Demoulin J B. FOXO transcription factors in cancer development and therapy. Cell Mol Life Sci 2016; 73(6):1159-72.

TABLE 4
Expression data of target genes in different samples
A: Colon

| | | | | | | | SOD2 | SOD2 | Foxo3 |
|---|---|---|---|---|---|---|---|---|---|
| | Colon | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 10- | Adenoma Active - Normal | 0.273798 | 0.269074 | −1.48303 | −3.13745 | −1.67616 | 0.836813 | 0.236897 | −0.25657 |
| 10 | active | | | | | | | | |
| 20- | Adenocarcinoma active - | 1.9587 | 1.710284 | −1.22683 | −3.37404 | −2.15956 | 1.85336 | 1.628937 | −0.34943 |
| 10 | normal active | | | | | | | | |

GSE37364, GSE39084, GSE40967, GSE2109, GSE4183, GSE8671, GSE1433, GSE20916

B: Breast cancer subtypes

| | | | | | | | SOD2 | SOD2 | Foxo3 |
|---|---|---|---|---|---|---|---|---|---|
| | Breast | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 15- | Basal Active - Normal active | 1.667574 | 1.863761 | −0.53552 | −1.13966 | −0.49688 | 3.044671 | 3.04274 | 0.351846 |
| 15 | | | | | | | | | |
| 15- | HER2 Active - Normal active | 1.299157 | 1.300141 | −1.06429 | −1.25087 | −0.59512 | 2.030035 | 2.445361 | 0.098959 |
| 15 | | | | | | | | | |
| 15- | LumA Active - Normal | 1.192817 | 1.04744 | −0.63432 | −0.97879 | −0.85001 | 1.13798 | 1.29578 | 0.115024 |
| 15 | active | | | | | | | | |
| 15- | LumB Active - Normal | 1.659292 | 1.666756 | −0.55229 | −1.06282 | −0.94702 | 1.844972 | 2.066248 | −0.15813 |
| 15 | active | | | | | | | | |
| 15- | NormL Active - Normal | 0.379788 | 0.199025 | −0.29916 | −0.1721 | −0.45272 | 0.873948 | 1.067476 | 0.495257 |
| 15 | active | | | | | | | | |

GSE6532, GSE10780, GSE12276, GSE21653, GSE26910, GSE42568, GSE45827, GSE58697, EMTAB365, GSE7307, GSE54002

C: Endometrioid endometrium (non-malignant)

| | | | | | | | SOD2 | SOD2 | Foxo3 |
|---|---|---|---|---|---|---|---|---|---|
| | Endometrium | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 5- | Grade 3 Endometrium | 1.056748 | 0.942018 | 0.218929 | 0.78093 | −0.53813 | −0.27305 | 0.115214 | 0.327355 |
| 10 | endometrioid Active - Normal | | | | | | | | |
| | active | | | | | | | | |
| 5- | Grade 2 Endometrium | 0.883182 | 0.515255 | −0.25033 | −0.29361 | −1.20879 | −0.54561 | −0.00248 | 0.321328 |
| 10 | endometrioid Active - Normal | | | | | | | | |
| | active | | | | | | | | |
| 5- | Grade 1 Endometrium | −0.46113 | −0.83605 | −0.43501 | 1.758022 | −0.84941 | −1.32275 | −0.93531 | 0.130646 |
| 10 | endometrioid Active - Normal | | | | | | | | |
| | active | | | | | | | | |

GSE65986, GSE56026, GSE51981, GSE39099, GSE29437, GSE29436, GSE20854, GSE17025, GSE7307, GSE7305, GSE4888, GSE3526

D: Bladder cancer

| | | | | | | | SOD2 | SOD2 | Foxo3 |
|---|---|---|---|---|---|---|---|---|---|
| | Bladder | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 15- | Urothelial cell carcinoma | 0.246742 | 0.634363 | −0.26445 | −0.05666 | −0.33966 | 1.536777 | 1.668267 | 0.08466 |
| 10 | Active - Normal bladder | | | | | | | | |
| | active | | | | | | | | |

GSE31684, GSE31189, GSE30522, GSE7476, GSE11839

E: Barrett's esophagus (non-malignant) and esophageal cancer

| | | | | | | | SOD2 | SOD2 | Foxo3 |
|---|---|---|---|---|---|---|---|---|---|
| | Esopahagael | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 5-8 | Barrett's esophagus active - | −1.16364 | −3.01546 | −1.25124 | 0.180396 | −0.52154 | −1.06208 | −0.42729 | −1.73071 |
| | Normal esophagus active | | | | | | | | |
| 5-8 | Esophageal adenocarcinoma | −2.56948 | −2.27944 | −1.23093 | 1.887236 | −0.59335 | 0.29449 | 0.752051 | −0.78151 |
| | Active - Normal esophagus | | | | | | | | |
| | active | | | | | | | | |
| 8-8 | Esophageal carcinoma ESCC | −0.12874 | −0.17861 | −0.42882 | −1.46369 | −1.52075 | 0.407411 | 0.966236 | −1.32294 |
| | Active - Normal esophagus | | | | | | | | |
| | active | | | | | | | | |
| 8-8 | Esophageal carcinoma unclear | 0.185132 | −1.55941 | −1.48575 | −1.44444 | −0.86866 | 0.311418 | 1.63532 | −2.8173 |
| | type Active - Normal | | | | | | | | |
| | esophagus active | | | | | | | | |

GSE26886, GSE32701, GSE42363, GSE45670, GSE7307, GSE40220, GSE14827, GSE17351, GSE33810

F: Brain tumors

| | | | | | | | SOD2 | SOD2 | Foxo3 |
|---|---|---|---|---|---|---|---|---|---|
| | Brain | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 15- | Astrocytoma Active - Normal | 0.146884 | 0.011696 | −0.12663 | −1.27425 | 0.15012 | 0.885691 | 1.335549 | −0.16905 |
| 15 | active | | | | | | | | |
| 15- | Ependymoma Active - | 1.366425 | 1.335516 | 0.200653 | −1.35681 | 1.754186 | 1.674704 | 1.819496 | −0.05665 |
| 15 | normal active | | | | | | | | |
| 15- | Glioblastoma Active - | 0.432824 | 0.588991 | −0.28271 | −0.90651 | −0.31045 | 2.443816 | 2.540678 | −0.3791 |
| 15 | normal active | | | | | | | | |
| 5- | Glioma Active - Normal | 1.025915 | 0.826212 | 0.235253 | −1.14941 | 0.273865 | 1.628326 | 1.999869 | −0.94771 |
| 15 | active | | | | | | | | |
| 5- | Oligoastrocytoma Active - | −0.39944 | −0.97413 | −0.86394 | −1.32875 | −0.29759 | 0.485354 | 0.862014 | −0.80434 |
| 15 | normal active | | | | | | | | |
| 15- | Medulloblastoma Active - | −0.3592 | −0.33424 | 0.353031 | −1.17693 | 0.374427 | −1.3727 | −1.36469 | −0.14824 |
| 15 | normal active | | | | | | | | |
| 15- | Meningioma Active - Normal | −1.88971 | −1.41137 | 0.044613 | −1.0159 | 0.284705 | 0.487543 | 0.844608 | 0.155723 |
| 15 | active | | | | | | | | |
| 7- | PNET Pediatric Active - | −0.46105 | −0.27148 | −0.19149 | 0.249276 | 0.796817 | −0.72253 | −0.52649 | −0.04015 |
| 15 | normal active | | | | | | | | |
| 10- | ETMR pediatric Active - | −1.05009 | −0.99756 | −0.7645 | −1.22548 | −0.08409 | −1.27778 | −1.03669 | −1.14498 |
| 15 | normal active | | | | | | | | |
| 4- | Atypical teratoid/rhabdoid | 0.127491 | 0.421646 | −0.11917 | −1.33723 | −0.06481 | −0.86283 | −0.73715 | −0.4772 |
| 15 | Active - Normal active | | | | | | | | |
| 15- | Adamantinomatous | −1.01281 | −0.92224 | −0.3828 | −0.09534 | −0.16296 | −0.20932 | −0.22166 | −0.99084 |
| 15 | craniopharyngioma Active - | | | | | | | | |
| | normal active | | | | | | | | |
| 6- | Malignant peripheral nerve | −1.69854 | −1.20554 | −1.58464 | 1.0569 | −2.07102 | −1.28197 | −1.39175 | 0.329751 |
| 15 | sheath Active - Normal | | | | | | | | |
| | active | | | | | | | | |
| 5- | Papilloma Active - Normal | −1.91215 | −1.26686 | −1.99798 | −1.52666 | 1.835479 | −0.75212 | −1.08724 | −0.68474 |
| 15 | active | | | | | | | | |

GSE50161, GSE16581, GSE44971, GSE15824, GSE19350, GSE68015, GSE11882, GSE7307, GSE4780, GSSE9438, GSE73066,
GSE5675, GSE33331, GSE22927
GSE53733, GSE45921, GSE50774, GSE34824, GSE36245, GSE13041, GSE43378, GSE4290, GSE73038, 37418, GSE67850,
GSE 12992, GSE74195, GSE50161,
GSE66354, GSE68015, GSE66354, GSE50385, GSE16155, GSE21687, GSE45437, GSE12141, GSE70678, GSE9832, GSE51455,
GSE19348, GSE16910, GSE18180

G: Lung tissues and carcinomas

| | | | | | | | SOD2 | SOD2 | Foxo3 |
|---|---|---|---|---|---|---|---|---|---|
| | Lung | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 15- | Lung adenocarcinoma | 0.862772 | 1.15117 | 0.146792 | 0.04439 | −1.46338 | 0.340791 | 0.537944 | −0.12852 |
| 10 | Active - Normal Lung active | | | | | | | | |
| 15- | Lung carcinoma Active - | 1.368504 | 1.523603 | 0.063306 | 0.834799 | −1.13767 | 0.080692 | 0.346262 | 0.115441 |
| 10 | Normal Lung active | | | | | | | | |
| 5- | Lung carcinoma basaloid | 1.412481 | 1.779473 | 0.477305 | 1.438382 | −1.55802 | −0.27336 | 0.093518 | 0.369853 |
| 10 | Active - Normal Lung active | | | | | | | | |
| 5- | Lung carcinoma carcinoid | 1.757095 | 1.681628 | −0.21827 | 2.739352 | 1.139666 | −2.50947 | −2.52514 | −0.51509 |
| 10 | Active - Normal Lung active | | | | | | | | |
| 10- | Lung carcinoma large cell | 1.52916 | 1.588956 | 0.191703 | 0.540628 | −0.43365 | 0.046743 | 0.240385 | −0.12804 |
| 10 | Active - Normal Lung active | | | | | | | | |
| 5- | Lung carcinoma Malignant | 0.248672 | −0.93022 | −1.03017 | 0.574851 | −1.55522 | −0.74182 | 0.055374 | −1.4961 |
| 10 | Pleural Mesothelioma | | | | | | | | |
| | Active - Normal Lung active | | | | | | | | |
| 15- | Lung carcinoma non small | 0.90711 | 1.028795 | −0.05802 | −0.02026 | −0.65816 | 0.071709 | 0.337043 | −0.20114 |
| 10 | cell Active - Normal Lung | | | | | | | | |
| | active | | | | | | | | |
| 5- | Lung carcinoma small cell | 1.938049 | 2.003627 | 0.354586 | 2.643471 | −1.56829 | −1.4894 | −0.88207 | −0.10044 |
| 10 | Active - Normal Lung active | | | | | | | | |
| 10- | Lung carcinoma squamous | 1.409648 | 1.776817 | 0.167414 | 0.212526 | −1.82582 | 0.411398 | 0.6163 | 0.487339 |
| 10 | cell Active - Normal Lung | | | | | | | | |
| | active | | | | | | | | |
| 15-5 | Lung adenocarcinoma | 0.181774 | 2.513496 | 0.921171 | −0.09567 | −2.33804 | 0.204362 | −0.10273 | 0.512917 |
| | Active - Normal lung | | | | | | | | |
| | Parenchyma active | | | | | | | | |
| 15-5 | Lung carcinoma Active - | 0.687506 | 2.885929 | 0.837686 | 0.694743 | −2.01234 | −0.05574 | −0.29441 | 0.756882 |
| | Normal lung Parenchyma | | | | | | | | |
| | active | | | | | | | | |
| 5-5 | Lung carcinoma basaloid | 0.731483 | 3.141799 | 1.251684 | 1.298326 | −2.43268 | −0.40979 | −0.54716 | 1.011294 |
| | Active - Normal lung | | | | | | | | |
| | Parenchyma active | | | | | | | | |
| 5-5 | Lung carcinoma carcinoid | 1.076097 | 3.043954 | 0.556111 | 2.599296 | 0.265004 | −2.6459 | −3.16581 | 0.12635 |
| | Active - Normal lung | | | | | | | | |
| | Parenchyma active | | | | | | | | |
| 10-5 | Lung carcinoma large cell | 0.848162 | 2.951282 | 0.966083 | 0.400572 | −1.30831 | −0.08969 | −0.40029 | 0.513398 |
| | Active - Normal lung | | | | | | | | |
| | Parenchyma active | | | | | | | | |
| 5-5 | Lung carcinoma Malignant | −0.43233 | 0.432109 | −0.25579 | 0.434795 | −2.42988 | −0.87825 | −0.5853 | −0.85466 |
| | Pleural Mesothelioma | | | | | | | | |
| | Active - Normal lung | | | | | | | | |
| | Parenchyma active | | | | | | | | |
| 15-5 | Lung carcinoma non small | 0.226112 | 2.39112 | 0.716357 | −0.16031 | −1.53282 | −0.06472 | −0.30363 | 0.440297 |
| | cell Active - Normal lung | | | | | | | | |
| | Parenchyma active | | | | | | | | |
| 5-5 | Lung carcinoma small cell | 1.257051 | 3.365953 | 1.128966 | 2.503415 | −2.44296 | −1.62583 | −1.52274 | 0.541004 |
| | Active-Normal lung | | | | | | | | |
| | Parenchyma active | | | | | | | | |
| 10-5 | Lung carcinoma squamous | 0.72865 | 3.139143 | 0.941793 | 0.07247 | −2.70048 | 0.274968 | −0.02438 | 1.12878 |
| | cell Active - Normal lung | | | | | | | | |
| | Parenchyma active | | | | | | | | |
| 15-5 | Lung adenocarcinoma | 1.199132 | 1.473595 | 0.073019 | 0.00497 | −0.07875 | 0.864836 | 1.319405 | −0.46545 |
| | Active - Normal trachea | | | | | | | | |
| | Active | | | | | | | | |
| 15-5 | Lung carcinoma Active - | 1.704864 | 1.846028 | −0.01047 | 0.795379 | 0.24696 | 0.604736 | 1.127722 | −0.22149 |
| | Normal trachea Active | | | | | | | | |
| 5-5 | Lung carcinoma basaloid | 1.748841 | 2.101898 | 0.403532 | 1.398963 | −0.17339 | 0.250685 | 0.874979 | 0.032925 |
| | Active - Normal trachea | | | | | | | | |
| | Active | | | | | | | | |
| 5-5 | Lung carcinoma carcinoid | 2.093455 | 2.004053 | −0.29204 | 2.699932 | 2.524299 | −1.98542 | −1.74368 | −0.85202 |
| | Active - Normal trachea | | | | | | | | |
| | Active | | | | | | | | |
| 10-5 | Lung carcinoma large cell | 1.86552 | 1.911381 | 0.117931 | 0.501208 | 0.950987 | 0.570787 | 1.021845 | −0.46497 |
| | Active - Normal trachea | | | | | | | | |
| | Active | | | | | | | | |
| 5-5 | Lung carcinoma Malignant | 0.585033 | −0.60779 | −1.10394 | 0.535431 | −0.17059 | −0.21778 | 0.836834 | −1.83303 |
| | Pleural Mesothelioma | | | | | | | | |
| | Active - Normal trachea | | | | | | | | |
| | Active | | | | | | | | |
| 15-5 | Lung carcinoma non small | 1.24347 | 1.351219 | −0.1318 | −0.05968 | 0.726472 | 0.595754 | 1.118503 | −0.53807 |
| | cell Active - Normal trachea | | | | | | | | |
| | Active | | | | | | | | |
| 5-5 | Lung carcinoma small cell | 2.27441 | 2.326052 | 0.280814 | 2.604051 | −0.18366 | −0.96536 | −0.10061 | −0.43737 |
| | Active - Normal trachea | | | | | | | | |
| | Active | | | | | | | | |
| 10-5 | Lung carcinoma squamous | 1.746008 | 2.099242 | 0.093641 | 0.173106 | −0.44118 | 0.935442 | 1.39776 | 0.15041 |
| | cell Active - Normal trachea | | | | | | | | |
| | Active | | | | | | | | |
| 15-8 | Lung adenocarcinoma | 1.328557 | 1.475149 | 0.23599 | 0.063051 | −0.42398 | 2.604758 | 2.681653 | −0.06196 |
| | Active - Normal large airway | | | | | | | | |
| | Active | | | | | | | | |
| 15-8 | Lung carcinoma Active - | 1.834289 | 1.847582 | 0.152505 | 0.85346 | −0.09827 | 2.344659 | 2.489971 | 0.182004 |
| | Normal large airway Active | | | | | | | | |
| 5-8 | Lung carcinoma basaloid | 1.878267 | 2.103452 | 0.566504 | 1.457043 | −0.51861 | 1.990608 | 2.237227 | 0.436416 |
| | Active - Normal large airway | | | | | | | | |
| | Active | | | | | | | | |
| 5-8 | Lung carcinoma carcinoid | 2.22288 | 2.005607 | −0.12907 | 2.758013 | 2.179072 | −0.2455 | −0.38143 | −0.44853 |
| | Active - Normal large airway | | | | | | | | |
| | Active | | | | | | | | |
| 10-8 | Lung carcinoma large cell | 1.994945 | 1.912935 | 0.280902 | 0.559289 | 0.60576 | 2.31071 | 2.384093 | −0.06148 |
| | Active - Normal large airway | | | | | | | | |
| | Active | | | | | | | | |
| 5-8 | Lung carcinoma Malignant | 0.714458 | −0.60624 | −0.94097 | 0.593512 | −0.51581 | 1.522143 | 2.199083 | −1.42954 |
| | Pleural Mesothelioma | | | | | | | | |
| | Active - Normal large airway | | | | | | | | |
| | Active | | | | | | | | |
| 15-8 | Lung carcinoma non small | 1.372895 | 1.352773 | 0.031176 | −0.0016 | 0.381245 | 2.335676 | 2.480751 | −0.13458 |
| | cell Active - Normal large | | | | | | | | |
| | airway Active | | | | | | | | |
| 5-8 | Lung carcinoma small cell | 2.403835 | 2.327606 | 0.443785 | 2.662132 | −0.52889 | 0.774565 | 1.261639 | −0.03387 |
| | Active - Normal large airway | | | | | | | | |
| | Active | | | | | | | | |
| 10-8 | Lung carcinoma squamous | 1.875433 | 2.100796 | 0.256613 | 0.231187 | −0.78641 | 2.675364 | 2.760008 | 0.553902 |
| | cell Active - Normal large | | | | | | | | |
| | airway Active | | | | | | | | |
| 15- | Lung adenocarcinoma | 0.627512 | 1.009811 | −0.20951 | −0.01811 | −0.05601 | 1.666712 | 1.958862 | −0.1256 |
| 15 | Active - Normal small | | | | | | | | |
| | airway Active | | | | | | | | |
| 15- | Lung carcinoma Active - | 1.133244 | 1.382243 | −0.29299 | 0.772296 | 0.2697 | 1.406613 | 1.76718 | 0.118366 |
| 15 | Normal small airway Active | | | | | | | | |
| 5- | Lung carcinoma basaloid | 1.177222 | 1.638113 | 0.121005 | 1.37588 | −0.15065 | 1.052562 | 1.514437 | 0.372778 |
| 15 | Active - Normal small | | | | | | | | |
| | airway Active | | | | | | | | |
| 5- | Lung carcinoma carcinoid | 1.521835 | 1.540268 | −0.57457 | 2.676849 | 2.547039 | −1.18355 | −1.10422 | −0.51217 |
| 15 | Active - Normal small | | | | | | | | |
| | airway Active | | | | | | | | |
| 10- | Lung carcinoma large cell | 1.293901 | 1.447597 | −0.1646 | 0.478125 | 0.973727 | 1.372664 | 1.661303 | −0.12512 |
| 15 | Active - Normal small | | | | | | | | |
| | airway Active | | | | | | | | |
| 5- | Lung carcinoma Malignant | 0.013413 | −1.07158 | −1.38647 | 0.512348 | −0.14785 | 0.584097 | 1.476292 | −1.49318 |
| 15 | Pleural Mesothelioma | | | | | | | | |
| | Active - Normal small | | | | | | | | |
| | airway Active | | | | | | | | |
| 15- | Lung carcinoma non small | 0.67185 | 0.887435 | −0.41432 | −0.08276 | 0.749212 | 1.39763 | 1.757961 | −0.19822 |
| 15 | cell Active - Normal small | | | | | | | | |
| | airway Active | | | | | | | | |
| 5- | Lung carcinoma small cell | 1.70279 | 1.862267 | −0.00171 | 2.580968 | −0.16092 | −0.16348 | 0.538849 | −0.09751 |
| 15 | Active - Normal small | | | | | | | | |
| | airway Active | | | | | | | | |
| 10- | Lung carcinoma squamous | 1.174388 | 1.635458 | −0.18889 | 0.150023 | −0.41844 | 1.737319 | 2.037218 | 0.490264 |
| 15 | cell Active - Normal small | | | | | | | | |
| | airway Active | | | | | | | | |

GSE63074, GSE30219, GSE28582, GSE50081, GSE77803, GSE31210, GSE33532, GSE51024, GSE19804, GSE10006, GSE19667,
GSE13933, GSE43346, GSE7307.

H: Prostate cancer

| | | | | | | | SOD2 | SOD2 | Foxo3 |
|---|---|---|---|---|---|---|---|---|---|
| | Prostate | BNIP3 | BNIP3 | MXI1 | PCK1 | PPARGC1A | 215223_- | 216841_- | 204132_- |
| Nr of samples: sub type - normal | | 201849_at | 201848_s_at | 202364_at | 208383_s_at | 219195_at | s_at | s_at | s_at |
| 10-5 | Prostate adenocarcinoma | 0.882 | 0.787338 | 0.270096 | 0.232785 | −0.75766 | 0.621324 | 0.444623 | −0.45114 |
| | Active - Normal prostate | | | | | | | | |
| | active | | | | | | | | |
| 15-5 | Prostate Carcinoma Active - | −0.89432 | −0.53046 | −0.00952 | 0.106124 | 0.196289 | −0.72156 | −1.10879 | −0.33564 |
| | Normal prostate active | | | | | | | | |
| 4-5 | Prostate carcinoma high | −0.84998 | −0.65283 | −0.62984 | −0.0771 | −1.79734 | −2.79272 | −1.79146 | −1.70778 |
| | grade Active - Normal | | | | | | | | |
| | prostate active | | | | | | | | |
| 5-5 | Prostate carcinoma | 0.766682 | 0.048211 | 0.3789 | 0.603609 | −0.20119 | −2.41498 | −2.09149 | −0.49558 |
| | metastatic Active - Normal | | | | | | | | |
| | prostate active | | | | | | | | |

GSE21887, GSE15392, GSE17482, GSE17906, GSE17951, GSE18676, GSE22606, GSE26910, GSE28403, GSE30304, GSE32967,
GSE32982, GSE3325, GSE33316, GSE34043, GSE40794, GSE45016, GSE46602, GSE56352, GSE73044, GSE9951, GSE2109,
GSE7307

TABLE 6
Selected FOXO target genes

| Gene | Probeset | References |
|---|---|---|
| AGRP | 207193_at | (Kim et al., 2006; Kitamura et al., 2006) |
| BCL2L11 | 1553096_s_at/1555372_at/1558143_a_at/ | (Dijkers et al., 2000a; Gilley et al., 2003) |
| | 208536_s_at/222343_at/225606_at | |
| BCL6 | 203140_at/215990_s_at | (Fernandez de Mattos et al., 2004; Tang et al., 2002) |
| BNIP3 | 201848_s_at/201849_at | (Mammucari et al., 2007; Zhao et al., 2007) |
| BTG1 | 1559975_at/200920_s_at/200921_s_at | (Bakker et al., 2004) |
| CAT | 201432_at/211922_s_at/215573_at | (Nemoto and Finkel, 2002) |
| CAV1 | 203065_s_at/212097_at | (Roy et al., 2008; van den Heuvel et al., 2005) |
| CCND1 | 208711_s_at/208712_at/214019_at | (Schmidt et al., 2002) |
| CCND2 | 200951_s_at/200952_s_at/200953_s_at/ | (Schmidt et al., 2002) |
| | 231259_s_at | |
| CCNG2 | 1555056_at/202769_at/202770_s_at/ | (Chen et al., 2006; Martinez-Gac et al., 2004) |
| | 211559_s_at/228081_at | |
| CDKN1A | 1555186_at/202284_s_at | (Nakae et al., 2003; Seoane et al., 2004) |
| CDKN1B | 209112_at | (Dijkers et al., 2000b; Medema et al., 2000; Stahl et al., 2002) |
| ESR1 | 205225_at/211233_x_at/211234_x_at/ | (Guo and Sonenshein, 2004) |
| | 211235_s_at/211627_x_at/215551_at/ | |
| | 215552_s_at/217190_x_at/207672_at | |
| FASLG | 210865_at/211333_s_at | (Brunet et al., 1999; Ciechomska et al., 2003) |
| FBXO32 | 225801_at/225803_at/225345_s_at/225328_at | (Sandri et al., 2004) |
| GADD45A | 203725_at | (Furukawa-Hibi, 2002; Tran et al., 2002) |
| INSR | 207851_s_at/213792_s_at/226212_s_at/ | (Puig and Tjian, 2005) |
| | 226216_at/226450_at | |
| MXI1 | 202364_at | (Delpuech et al., 2007) |
| NOS3 | 205581_s_at | (Potente et al., 2005) |
| PCK1 | 208383_s_at | (Sekine et al., 2007) |
| POMC | 205720_at | (Harada et al., 2006; Kim et al., 2006) |
| PPARGC1A | 1569141_a_at/219195_at | (Daitoku et al., 2003) |
| PRDX3 | 201619_at/209766_at | (Chiribau et al., 2008) |
| RBL2 | 212331_at/212332_at | (Chen et al., 2006; Kops et al., 2002b) |
| SOD2 | 215078_at/215223_s_at/216841_s_at/ | (Kops et al., 2002a) |
| | 221477_s_at | |
| TNFSF10 | 202687_s_at/202688_at/214329_x_at | (Modur et al., 2002) |

TABLE 7
SOD2 and BNIP3 expression leves in various normal and tumor types

| | | | | | | | | |
|---|---|---|---|---|---|---|---|---|
| | | | | BNIP3 Probe | BNIP3 Probe | SOD2 Probe | SOD2 Probe | FOXO3 |
| | | Samples | | average | average | average | average | average |
| | Total | used to | | intensity and | intensity | intensity | intensity | intensity |
| | Nr. of | calculate | | StDv | and StDv | and StDv | and StDv | and StDv |
| GEO dataset | samples | average | Tissue type | 201849_at | 201848_s_at | 215223_s_at | 216841_s_at | 204132_s_at |

A:

| | | | | | | | | |
|---|---|---|---|---|---|---|---|---|
| GSE37364, GSE39084, | 121 | 10 | Normal colon active | 6.437889 | 5.869242 | 8.542018 | 7.84363 | 8.588602 |
| GSE40967, GSE2109, | | | | 0.447166 | 0.373749 | 0.446035 | 0.537961 | 0.372467 |
| GSE4183, GSE8671, | 67 | 10 | Colon adenoma | 6.711687 | 6.138316 | 9.378831 | 8.080526 | 8.332033 |
| GSE1433, GSE20916 | | | active | 1.426655 | 1.252235 | 1.461469 | 1.439315 | 0.395177 |
| | 1394 | 20 | Colon | 8.396589 | 7.579526 | 10.39538 | 9.472567 | 8.239174 |
| | | | adenocarcinoma | 1.112439 | 1.126591 | 0.881171 | 0.847033 | 0.514963 |
| | | | active | | | | | |
| GSE6532, GSE10780, | 161 | 15 | Normal breast active | 8.319051 | 7.357265 | 7.219094 | 5.77076 | 8.241385 |
| GSE12276, GSE21653, | | | | 0.28339 | 0.27218 | 0.553115 | 0.533596 | 0.764766 |
| GSE26910, GSE42568, | 440 | 15 | Luminal A breast | 9.511868 | 8.404704 | 8.357074 | 7.06654 | 8.356409 |
| GSE45827, GSE58697, | | | cancer active | 0.800135 | 0.925308 | 0.823774 | 0.792626 | 0.398693 |
| EMTAB365, GSE7307, | 325 | 15 | Luminal B breast | 9.978344 | 9.024021 | 9.064066 | 7.837008 | 8.083252 |
| GSE54002 | | | cancer active | 0.663878 | 0.779164 | 0.637656 | 0.728711 | 0.885695 |
| | 159 | 15 | HER2 breast cancer | 9.618208 | 8.657405 | 9.249129 | 8.216121 | 8.340344 |
| | | | active | 1.07878 | 1.224734 | 0.860431 | 0.980804 | 0.548851 |
| | 233 | 15 | Basal like breast | 9.316237 | 8.305644 | 10.26377 | 8.8135 | 8.593231 |
| | | | cancer active | 0.874281 | 0.897085 | 0.92037 | 0.825788 | 0.566783 |
| GSE31684, GSE31189, | 43 | 10 | Normal bladder | 8.577845 | 6.271738 | 8.365665 | 8.032263 | 6.757359 |
| GSE30522, GSE7476, | | | active | 0.607024 | 0.894274 | 1.405524 | 2.304808 | 1.061791 |
| GSE11839 | 145 | 15 | Urothelial cell | 8.824586 | 6.906101 | 9.902442 | 9.70053 | 6.842019 |
| | | | carcinoma active | 0.984354 | 1.109759 | 1.550475 | 1.682397 | 0.963735 |
| GSE26886, GSE32701, | 42 | 8 | Normal esophagus | 9.511993 | 8.643755 | 9.754773 | 8.51123 | 8.663209 |
| GSE42363, GSE45670, | | | active | 0.521287 | 0.556569 | 0.805589 | 0.655757 | 0.585179 |
| GSE7307, GSE40220, | 20 | 5 | Barrett's esophagus | 8.348352 | 5.628293 | 8.692696 | 8.083941 | 6.932503 |
| GSE14827, | | | active | 1.608622 | 1.997879 | 0.954547 | 0.973802 | 0.327309 |
| GSE17351, GSE33810 | 45 | 8 | Esophageal | 9.383256 | 8.465146 | 10.16218 | 9.477466 | 7.340267 |
| | | | Squamous cell | 1.483722 | 1.511957 | 0.82945 | 0.806687 | 1.020953 |
| | | | carcinoma active | | | | | |
| | 35 | 5 | Esophageal | 6.942516 | 6.364311 | 10.04926 | 9.263281 | 7.8817 |
| | | | adenocarcinoma | 1.183051 | 0.832998 | 1.028668 | 0.987494 | 0.361725 |
| | | | active | | | | | |
| GSE50161, GSE16581, | | 15 | Normal brain active | 10.571 | 9.165685 | 8.420073 | 7.401808 | 8.411185 |
| GSE44971, GSE15824, | | | | 0.93017 | 0.918522 | 1.815166 | 2.073445 | 0.742852 |
| GSE19350, GSE68015, | | 15 | Astrocytoma active | 10.79248 | 9.2328 | 8.983583 | 8.608461 | 8.04843 |
| GSE11882, GSE7307, | | | | 0.954059 | 1.07454 | 1.271324 | 1.514669 | 0.540728 |
| GSE4780, GSSE9438, | | 15 | Ependymoma active | 11.93806 | 10.55883 | 10.10958 | 9.270556 | 7.992423 |
| GSE73066, GSE5675, | | | | 0.426621 | 0.467185 | 1.631254 | 1.613944 | 0.785697 |
| GSE33331, GSE22927, | | 5 | Glioma active | 11.60068 | 10.003 | 10.38833 | 9.938395 | 7.217868 |
| GSE53733, GSE45921, | | | | 0.513624 | 0.737599 | 0.873492 | 1.100576 | 0.627584 |
| GSE50774, GSE34824, | | 15 | Glioblastoma active | 11.21654 | 10.02933 | 10.80697 | 9.990391 | 7.959677 |
| GSE36245, GSE13041, | | | | 0.733984 | 0.901195 | 0.949478 | 0.960017 | 0.600796 |
| GSE43378, GSE4290, | | | | | | | | |
| GSE73038, GSE37418, | | | | | | | | |
| GSE67850, GSE12992, | | | | | | | | |
| GSE74195, GSE50161, | | | | | | | | |
| GSE66354, GSE68015, | | | | | | | | |
| GSE66354, GSE50385, | | | | | | | | |
| GSE16155, GSE21687, | | | | | | | | |
| GSE45437, | | | | | | | | |
| GSE12141, GSE70678, | | | | | | | | |
| GSE9832, GSE51455, | | | | | | | | |
| GSE19348, GSE16910, | | | | | | | | |
| GSE18180 | | | | | | | | |

B:

| | | | | | | | | |
|---|---|---|---|---|---|---|---|---|
| GSE37364, GSE39084, | 121 | 114 | Normal colon active | 6.766300 | 6.026765 | 8.196375983 | 6.995536285 | 8.415143741 |
| GSE40967, GSE2109, | | | | 0.551473 | 0.507432 | 0.570831 | 0.790092 | 0.431592 |
| GSE4183, GSE8671, | 76 | 12 | Colon adenoma | 6.904867 | 6.305086 | 9.439642059 | 8.100363041 | 8.30777867 |
| GSE1433, GSE20916 | | | active | 1.383773 | 1.205611 | 1.370847 | 1.318199 | 0.368975 |
| | 1341 | 450 | Colon | 7.417266 | 6.773805 | 9.715393514 | 8.650113556 | 8.014610253 |
| | | | adenocarcinoma | 1.412652 | 1.18832 | 0.918502 | 0.955991 | 0.701335 |
| | | | active | | | | | |
| GSE6532, GSE10780, | 175 | 149 | Normal breast active | 8.580632 | 7.502588 | 7.105725 | 5.838791 | 8.122807 |
| GSE12276, GSE21653, | | | | 0.705929 | 0.623842 | 0.763677 | 0.945085 | 0.818499 |
| GSE26910, GSE42568, | 624 | 536 | Luminal A breast | 8.967998 | 7.772701 | 7.603739 | 6.430053 | 8.185212 |
| GSE45827, GSE58697, | | | cancer active | 0.74231 | 0.693106 | 0.715028 | 0.835848 | 0.603764 |
| EMTAB365, GSE7307, | 486 | 304 | Luminal B breast | 9.394412 | 8.228799 | 8.127528 | 6.954036 | 8.048243 |
| GSE54002 | | | cancer active | 0.934612 | 0.950355 | 0.875258 | 0.952059 | 0.784529 |
| | 267 | 206 | HER2 breast cancer | 9.306159 | 8.168413 | 8.580925 | 7.311002 | 8.275227 |
| | | | active | 0.967564 | 1.029658 | 0.846655 | 1.018047 | 0.636811 |
| | 319 | 255 | Basal like breast | 9.289813 | 8.300672 | 9.710912 | 8.375586 | 8.36859 |
| | | | cancer active | 1.065399 | 1.082328 | 1.095402 | 1.186299 | 0.665996 |
| | 43 | 10 | Normal bladder | 8.578437 | 6.272345 | 8.365931 | 8.03247 | 6.758705 |
| | | | active | 0.607002 | 0.893837 | 1.40522 | 2.303875 | 1.060427 |
| GSE31684, GSE31189, | 145 | 34 | Urothelial cell | 8.376082 | 6.953712 | 9.394505 | 8.772433 | 6.779379 |
| GSE30522, GSE7476, | | | carcinoma | 1.336647 | 1.230363 | 1.605892 | 2.090078 | 0.935848 |
| GSE11839 | | | active | | | | | |
| GSE26886, GSE32701, | 42 | 35 | Normal esophagus | 10.31495 | 8.398459 | 9.566248 | 8.839943 | 7.529135 |
| GSE42363, GSE45670, | | | active | 0.521287 | 0.746881 | 0.805589 | 1.158115 | 0.910611 |
| GSE7307, GSE40220, | 20 | 4 | Barrett's esophagus | 8.721536 | 5.915304 | 8.924126 | 8.287561 | 7.061635 |
| GSE14827, GSE17351, | | | active | 1.608622 | 2.184687 | 0.954547 | 0.993971 | 0.177972 |
| GSE33810. | 44 | 37 | Esophageal | 9.907335 | 8.888424 | 9.80937 | 9.353739 | 6.88251 |
| | | | Squamous cell | 1.483722 | 1.134621 | 0.82945 | 0.609105 | 0.741576 |
| | | | carcinoma active | | | | | |
| | 35 | 12 | Esophageal | 6.511577 | 5.959288 | 9.744908 | 9.155751 | 7.734009 |
| | | | adenocarcinoma | 1.183051 | 1.352522 | 1.028668 | 0.942176 | 0.77448 |
| | | | active | | | | | |
| GSE50161, GSE16581, | 515 | 73 | Normal brain active | 10.69179 | 9.183911 | 7.41698 | 6.147368 | 8.298635 |
| GSE44971, GSE15824, | | | | 0.823843 | 0.938014 | 1.410068 | 1.682804 | 0.619204 |
| GSE19350, GSE68015, | 221 | 94 | Astrocytoma active | 10.42371 | 8.827601 | 8.171173 | 7.642736 | 7.760604 |
| GSE11882, GSE7307, | | | | 0.697635 | 0.752459 | 1.309707 | 1.45043 | 0.691973 |
| GSE4780, GSSE9438, | 120 | 50 | Ependymoma active | 11.18481 | 9.983857 | 9.51132 | 8.602994 | 7.933142 |
| GSE73066, GSE5675, | | | | 0.801885 | 0.68127 | 1.496878 | 1.529031 | 0.797787 |
| GSE53733, GSE45921, | 39 | 20 | Glioma active | 10.93251 | 9.44152 | 8.81291 | 8.088357 | 7.554503 |
| GSE50774, GSE34824, | | | | 0.935365 | 1.044345 | 1.891867 | 1.994029 | 0.640079 |
| GSE36245, GSE13041, | 289 | 179 | Glioblastoma active | 10.66393 | 9.005034 | 9.234986 | 8.592844 | 7.662142 |
| GSE43378, GSE4290, | | | | 0.807012 | 1.162699 | 1.331689 | 1.434318 | 0.889288 |
| GSE73038, GSE37418, | | | | | | | | |
| GSE67850, GSE12992, | | | | | | | | |
| GSE66354, GSE68015, | | | | | | | | |
| GSE66354, GSE50385, | | | | | | | | |
| GSE16155, GSE21687, | | | | | | | | |
| GSE45437, GSE12141, | | | | | | | | |
| GSE70678, GSE9832, | | | | | | | | |
| GSE51455, GSE19348, | | | | | | | | |
| GSE33331, GSE22927, | | | | | | | | |
| GSE74195, GSE50161, | | | | | | | | |
| GSE16910, GSE18180 | | | | | | | | |

### SEQUENCE LISTING

| Seq. No.: | Gene: |
|---|---|
| Seq. 1 | AGRP |
| Seq. 2 | ATG14 |
| Seq. 3 | ATP8A1 |
| Seq. 4 | BCL2L11 |
| Seq. 5 | BCL6 |
| Seq. 6 | BIRC5 |
| Seq. 7 | BNIP3 |
| Seq. 8 | BTG1 |
| Seq. 9 | C10orf10 |
| Seq. 10 | CAT |
| Seq. 11 | CAV1 |
| Seq. 12 | CBLB |
| Seq. 13 | CCND1 |
| Seq. 14 | CCND2 |
| Seq. 15 | CCNG2 |
| Seq. 16 | CDKN1A |
| Seq. 17 | CDKN1B |
| Seq. 18 | DDB1 |
| Seq. 19 | DYRK2 |
| Seq. 20 | ERBB3 |
| Seq. 21 | EREG |
| Seq. 22 | ESR1 |
| Seq. 23 | EXT1 |
| Seq. 24 | FASLG |
| Seq. 25 | FBXO32 |
| Seq. 26 | FGFR2 |
| Seq. 27 | GADD45A |
| Seq. 28 | IGF1R |
| Seq. 29 | IGFBP1 |
| Seq. 30 | IGFBP3 |
| Seq. 31 | INSR |
| Seq. 32 | KLF2 |
| Seq. 33 | KLF4 |
| Seq. 34 | LGMN |
| Seq. 35 | MXI1 |
| Seq. 36 | MYOD1 |
| Seq. 37 | NOS3 |
| Seq. 38 | PCK1 |
| Seq. 39 | PDK4 |
| Seq. 40 | POMC |
| Seq. 41 | PPARGC1A |
| Seq. 42 | PPM1D |
| Seq. 43 | PRDX3 |
| Seq. 44 | RAG1 |
| Seq. 45 | RAG2 |
| Seq. 46 | RBL2 |
| Seq. 47 | SEMA3C |
| Seq. 48 | SEPPI |
| Seq. 49 | SESN1 |
| Seq. 50 | SIRT1 |
| Seq. 51 | SLC5A3 |
| Seq. 52 | SMAD4 |
| Seq. 53 | SOD2 |
| Seq. 54 | STK11 |
| Seq. 55 | TLE4 |
| Seq. 56 | TNFSF10 |
| Seq. 57 | TXNIP |

1. Method for inferring the activity of a PI3K cellular signaling pathway in a subject based at least on the oxidative stress state of the subject, preferably the oxidative stress state of a FOXO transcription factor element in the subject, comprising,
inferring the oxidative stress state of the subject, preferably the oxidative stress state of the FOXO transcription factor element in the subject, based on the expression level(s) of one or more gene(s) selected from the group consisting of SOD2, BNIP3, MXI1, PCK1, PPARGC1A and CAT in the subject.

2. Method according to claim 1, wherein said inferring the activity of the PI3K cellular signaling pathway in the subject is based on the inferred oxidative stress state of the subject and the activity level of the FOXO transcription factor element in the subject.

3. Method according to claim 1 or 2, further comprising determining the expression level(s) of the one or more genes in the subject.

4. Method according to claim 1, wherein the inferring the oxidative stress state of the subject is based on the expression level(s) of at least four, preferably all of the FOXO target gene(s) selected from the group consisting of SOD2, BNIP3, MXI1, PCK1, PPARGC1A and CAT in the subject.

5. Method according to claim 3, wherein the inferring the oxidative stress state of the subject is based on the expression level(s) of two or more, preferably all of the FOXO target gene(s) selected from the group consisting of of SOD2, BNIP3, MXI1 and PCK1 in the subject.

6. Method according to claim 3, wherein the inferring the oxidative stress state of the subject is based on the expression level of one FOXO target gene selected from the group consisting of SOD2, BNIP3, MXI1, PCK1, PPARGC1A and CAT in the subject.

7. Method according to claim 4, wherein an oxidative stress state is inferred, when the expression level(s) of SOD2 and/or BNIP3 are upregulated in an extracted sample of the subject compared to a control sample and/or when the expression level(s) of one or more target gene(s) selected from MXI1, PCK1, PPARGC1A and CAT are downregulated in an extracted sample of the subject compared to a control sample.

8. Method according to claim 1, wherein the activity level of the FOXO transcription factor element in the subject is determined based at least on expression levels of one or more, preferably at least three, target gene(s) of the PI3K cellular signaling pathway measured in an extracted sample of the subject selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10.

9. Method according to claim 8, wherein the activity level of the FOXO transcription factor element is determined further based at least on expression levels of one or more, preferably at least three, target gene(s) of the PI3K cellular signaling pathway measured in the extracted sample of the subject selected from the group consisting of: ATP8A1, C10orf10, CBLB, DDB1, DYRK2, ERBB3, EREG, EXT1, FGFR2, IGF1R, IGFBP1, IGFBP3, LGMN, PPM1D, SEMA3C, SEPP1, SESN1, SLC5A3, SMAD4 and TLE4 and/or selected from the group consisting of: ATG14, BIRC5, IGFBP1, KLF2, KLF4, MYOD1, PDK4, RAG1, RAG2, SESN1, SIRT1, STK11 and TXNIP.

10. Method according to claim 1, further comprising:
determining whether the PI3K cellular signalling pathway is operating abnormally in the subject based on the inferred activity of the PI3K cellular signalling pathway in the subject.

11. Method according to claim 10, further comprising:
recommending prescribing a drug for the subject that corrects for abnormal operation of the PI3K cellular signalling pathway, wherein the recommending is performed if the PI3K cellular signalling pathway is determined to be operating abnormally in the subject based on the inferred activity of the PI3K cellular signalling pathway.

12. Method according to claim 1, wherein the method is used to indicate a cancer or pre-cancer state in a subject.

13. Method according to claim 12, wherein the method is used in at least one of the following activities:
diagnosis based on the inferred activity of the PI3K cellular signalling pathway in the subject;
prognosis based on the inferred activity of the PI3K cellular signalling pathway in the subject;
drug prescription based on the inferred activity of the PI3K cellular signalling pathway in the subject;
prediction of drug efficacy based on the inferred activity of the PI3K cellular signalling pathway in the subject;
prediction of adverse effects based on the inferred activity of the PI3K cellular signalling pathway in the subject;
monitoring of drug efficacy;
drug development;
assay development;
pathway research;
cancer staging;
enrollment of the subject in a clinical trial based on the inferred activity of the PI3K cellular signalling pathway in the subject;
selection of subsequent test to be performed; and
selection of companion diagnostics tests.

14. A computer program comprising program code means for causing a digital processing device to perform the method according to claim 13.
